SEQUENCE LISTING (106 sequences)

SEQ ID NO: 1 - protein, 179 aa, Homo sapiens
ADRERSIHDFCLVSKVVGRCRASMPRWWYNVTDGSCQLFVYGGCDGNSNNYLTKEECLKKCATVTENATGDLATSRNAADSSVPSAPRRQDSEDHSSDMFNYEEYCTANAVTGPCRASFPRWYFDVERNSCNNFIYGGCRGNKNSYRSEEACMLRCFRQQENPPLPLGSKVVVLAGAVS

SEQ ID NO: 2 - protein, 197 aa, Homo sapiens
  SIGNAL (1)..(18)
AGSFLAWLGSLLLSGVLAADRERSIHDFCLVSKVVGRCRASMPRWWYNVTDGSCQLFVYGGCDGNSNNYLTKEECLKKCATVTENATGDLATSRNAADSSVPSAPRRQDSEDHSSDMFNYEEYCTANAVTGPCRASFPRWYFDVERNSCNNFIYGGCRGNKNSYRSEEACMLRCFRQQENPPLPLGSKVVVLAGAVS

SEQ ID NO: 3 - protein, 153 aa, Homo sapiens
IHDFCLVSKVVGRCRASMPRWWYNVTDGSCQLFVYGGCDGNSNNYLTKEECLKKCATVTENATGDLATSRNAADSSVPSAPRRQDSEDHSSDMFNYEEYCTANAVTGPCRASFPRWYFDVERNSCNNFIYGGCRGNKNSYRSEEACMLRCFRQ

SEQ ID NO: 4 - protein, 58 aa, Homo sapiens
IHDFCLVSKVVGRCRASMPRWWYNVTDGSCQLFVYGGCDGNSNNYLTKEECLKKCATV

SEQ ID NO: 5 - protein, 51 aa, Homo sapiens
CLVSKVVGRCRASMPRWWYNVTDGSCQLFVYGGCDGNSNNYLTKEECLKKC

SEQ ID NO: 6 - protein, 58 aa, Homo sapiens
YEEYCTANAVTGPCRASFPRWYFDVERNSCNNFIYGGCRGNKNSYRSEEACMLRCFRQ

SEQ ID NO: 7 - protein, 51 aa, Homo sapiens
CTANAVTGPCRASFPRWYFDVERNSCNNFIYGGCRGNKNSYRSEEACMLRC

SEQ ID NO: 8 - protein, 92 aa, Homo sapiens
ADRERSIHDFCLVSKVVGRCRASMPRWWYNVTDGSCQLFVYGGCDGNSNNYLTKEECLKKCATVTENATGDLATSRNAADSSVPSAPRRQDS

SEQ ID NO: 9 - DNA, 708 bp, Artificial Sequence
  Consensus DNA sequence of human Bikunin (Fig. 3).
  misc_feature (622)..(622): “n” is any nucleotide.
  misc_feature (679)..(679): “n” is any nucleotide.
  misc_feature (707)..(707): “n” is any nucleotide.
ggccgggtcgtttctcgcctggctgggatcgctgctcctctctggggtcctggcggccgaccgagaacgcagcatccacgacttctgcctggtgtcgaaggtggtgggcagatgccgggcctccatgcctaggtggtggtacaatgtcactgacggatcctgccagctgtttgtgtatgggggctgtgacggaaacagcaataattacctgaccaaggaggagtgcctcaagaaatgtgccactgtcacagagaatgccacgggtgacctggccaccagcaggaatgcagcggattcctctgtcccaagtgctcccagaaggcaggattctgaagaccactccagcgatatgttcaactatgaagaatactgcaccgccaacgcagtcactgggccttgccgtgcatccttcccacgctggtactttgacgtggagaggaactcctgcaataacttcatctatggaggctgccggggcaataagaacagctaccgctctgaggaggcctgcatgctccgctgcttccgccagcaggagaatcctcccctgccccttggctcaaaggtggtggttctggccggggctgtttcgtgatggtgttgatccttttcctggggagcntccatggtcttactgattccgggtggcaaggaggaaccaggagcgtgccctgcggancgtctggagcttcggagatgacaagggnt

SEQ ID NO: 10 - protein, 197 aa, Artificial Sequence; identical to SEQ ID NO: 2
  Amino acids -18 to 179 of the translation of the consensus DNAsequence in Fig. 3.

SEQ ID NO: 11 - protein, 179 aa, Artificial Sequence
  Variants of human Bikunin.
  MISC_FEATURE (8)..(8): Each “Xaa” independently represents a naturally occurring amino acid residue except Cys, with the proviso that at least one “Xaa” in SEQ ID NO11 is different from the corresponding amino acid residue of the native sequence.
  MISC_FEATURE (17)..(17): Each “Xaa” independently represents a naturally occurring aminoacid residue except Cys, with the proviso that at least one “Xaa” in SEQ ID NO11 is different from the corresponding amino acid residue of the native sequence.
  MISC_FEATURE (19)..(19): Each “Xaa” independently represents a naturally occurring aminoacid residue except Cys, with the proviso that at least one “Xaa” in SEQ ID NO11 is different from the corresponding amino acid residue of the native sequence.
  MISC_FEATURE (21)..(26): Each “Xaa” independently represents a naturally occurring aminoacid residue except Cys, with the proviso that at least one “Xaa” in SEQ ID NO11 is different from the corresponding amino acid residue of the native sequence.
  MISC_FEATURE (40)..(40): Each “Xaa” independently represents a naturally occurring aminoacid residue except Cys, with the proviso that at least one “Xaa” in SEQ ID NO11 is different from the corresponding amino acid residue of the native sequence.
  MISC_FEATURE (42)..(42): 11 is different from the corresponding amino acid residue of the native sequence.
  MISC_FEATURE (45)..(47): Each “Xaa” independently represents a naturally occurring aminoacid residue except Cys, with the proviso that at least one “Xaa” in SEQ ID NO11 is different from the corresponding amino acidresidue of the native sequence.
  MISC_FEATURE (52)..(52): 11 is different from the corresponding amino acid residue of the native sequence.
  MISC_FEATURE (64)..(64): Each “Xaa” independently represents a naturally occurring aminoacid residue except Cys, with the proviso that at least one “Xaa” in SEQ ID NO11 is different from the corresponding amino acid residue of the native sequence.
  MISC_FEATURE (103)..(103): Each “Xaa” independently represents a naturally occurring aminoacid residue except Cys, with the proviso that at least one “Xaa” in SEQ ID NO11 is different from the corresponding amino acid residue of the native sequence.
  MISC_FEATURE (112)..(112): Each “Xaa” independently represents a naturally occurring aminoacid residue except Cys, with the proviso that at least one “Xaa” in SEQ ID NO11 is different from the corresponding amino acid residue of the native sequence.
  MISC_FEATURE (114)..(114): Each “Xaa” independently represents a naturally occurring aminoacid residue except Cys, with the proviso that at least one “Xaa” in SEQ ID NO11 is different from the corresponding amino acid residue of the native sequence.
  MISC_FEATURE (116)..(121): Each “Xaa” independently represents a naturally occurring aminoacid residue except Cys, with the proviso that at least one “Xaa” in SEQ ID NO11 is different from the corresponding amino acid residue of the native sequence.
  MISC_FEATURE (135)..(135): Each “Xaa” independently represents a naturally occurring aminoacid residue except Cys, with the proviso that at least one “Xaa” in SEQ ID NO11 is different from the corresponding amino acid residue of the native sequence.
  MISC_FEATURE (137)..(137): Each “Xaa” independently represents a naturally occurring aminoacid residue except Cys, with the proviso that at least one “Xaa” in SEQ ID NO11 is different from the corresponding amino acid residue of the native sequence.
  MISC_FEATURE (140)..(142): Each “Xaa” independently represents a naturally occurring aminoacid residue except Cys, with the proviso that at least one “Xaa” in SEQ ID NO11 is different from the corresponding amino acid residue of the native sequence.
  MISC_FEATURE (147)..(147): Each “Xaa” independently represents a naturally occurring aminoacid residue except Cys, with the proviso that at least one “Xaa” in SEQ ID NO11 is different from the corresponding amino acid residue of the native sequence.
  MISC_FEATURE (159)..(159): Each “Xaa” independently represents a naturally occurring aminoacid residue except Cys, with the proviso that at least one “Xaa” in SEQ ID NO11 is different from the corresponding amino acid residue of the native sequence.
ADRERSIXDFCLVSKVXGXCXXXXXXWWYNVTDGSCQLFXYXGCXXXSNNYXTKEECLKKCATXTENATGDLSTSRNAADSSVPSAPRRQDSEHDSSDMFNYXEYCTANAVXGXCXXXXXXWYFDVERNSCNNFXYXGCXXXKNSYXSEEACMLRCFRXQENPPLPLGSKVVVLAGAVS

SEQ ID NO: 12 - DNA, 393 bp, Homo sapiens
  misc_feature (361)..(361): “n” is any nucleotide.
  misc_feature (367)..(367): “n” is any nucleotide.
  misc_feature (384)..(384): “n” is any nucleotide.
  misc_feature (390)..(390): “n” is any nucleotide.
ggccgggtcgtttctcgcctggctgggatcgctgctcctctctggggtcctggccggccgaccgagaacgcagcatccacgacttctgcctggtgtcgaaggtggtgggcagattccgggcctccatgcctaggtggtggtacaatgtcactgacggatcctgccagctgtttgtgtatgggggctgtgacggaaacagcaataattacctgaccaaggaggagtgcctcaagaaatgtgccactgtcacagagaatgccacgggtgacctggccaccagcaggaatgcagcggattcctctgtcccaagtgctcccagaaggcaggattcttgaagaccacttcagcgatatgtttcaantattgnaagaataattgcaccgncaacgnatt

SEQ ID NO: 13 - protein, 110 aa, Homo sapiens
  SIGNAL (1)..(18)
PGRFSPGWDRCSSLGSWPADRERSIHDFCLVSKVVGRFRASMPRWWYNVTDGSCQLFVYGGCDGNSNNYLTKEECLKKCATVTENATGDLATSRNAADSSVPSAPRRQDS

SEQ ID NO: 14 - DNA, 510 bp, Homo sapiens
  misc_feature (424)..(424): “n” is any nucleotide.
  misc_feature (481)..(481): “n” is any nucleotide.
  misc_feature (509)..(509): “n” is any nucleotide.
gcaataattacctgaccaaggaggagtgcctcaagaaatgtgccactgtcacagagaatgccacgggtgacctggccaccagcaggaatgcagcggattcctctgtcccaagtctcccagaaggcaggattctgaagaccactccagcgatatgttcaactatgaagaatactgcaccgccaacgcagtcactgggccttgccgtgcatccttcccacgctggtactttgacgtggagaggaactcctgcaataacttcatctatggaggctgccggggcaataagaacagctaccgctctgaggaggcctgcatgctccgctgcttccgccagcaggagaatcctcccctgccccttggctcaaaggtggtggttctggccggggctgtttcgtgatggtgttgatccttttcctggggagcntccatggtcttactgattccgggtggcaaggaggaaccaggagcgtgccctgcggancgtctggagcttcggagatgacaagggnt

SEQ ID NO: 15 - protein, 20 aa, Homo sapiens
LPDQGGVPQEMCHCHRECHG

SEQ ID NO: 16 - DNA, 427 bp, Homo sapiens
  misc_feature (3)..(3): “n” is any nucleotide.
  misc_feature (11)..(12): “n” is any nucleotide.
  misc_feature (17)..(17): “n” is any nucleotide.
  misc_feature (48)..(48): “n” is any nucleotide.
  misc_feature (425)..(425): “n” is any nucleotide.
gcngcgcgttnntcgcntgctgggatcgctgcacctctctggggtcgnggcggccgaccgagaacgcagcatccacgacttctgcctggtgtcgaaggtggtgggcagatgccgggcctccatgcctaggtggtggtacaatgtcactgacggatcctgccagctgtttgtgtatgggggctgtgacggaaacagcaataattacctgaccaaggaggagtgcctcaagaaatgtgccactgtcacagagaatgccacgggtgacctggccaccagcaggaatgcagcggattcctctgtcccaagtgctcccagaaggcaggattctgaagaccactccagcgatatgttcaactatgaagaatactggcaccgccaacgcattcactgggcctgcgtgcatccttcccacgctggtactttgncg

SEQ ID NO: 17 - DNA, 423 bp, Homo sapiens
  misc_feature (6)..(6): “n” is any nucleotide.
  misc_feature (401)..(401): “n” is any nucleotide.
  misc_feature (407)..(407): “n” is any nucleotide.
tgggantcgctgctcctctctggggtcctggcggccgaccgagaacgcagcatccacgacttctgcctggtgtcgaaggtggtgggcagatgccgggcctccatgcctaggtggtggtacaatgtcactgacggatcctgccagctgtttgtgtatgggggctgtgacggaaacagcaataattacctgaccaaggaggagtgcctcaagaaatgtgccactgtcacagagaatgccacgggtgacctggccaccagcaggaatgcagcggattcctctgtcccaagtgctcccagaaggcaggattctgaagaccactccagcgatatgttcaactatgaagaatactgcaccgccaacgcagtcactgggccttgcgtggaatcctttcccacgctggnaatttngacgttgagaaggaac

SEQ ID NO: 18 - protein, 57 aa, Unknown
  Kunitz-like domain of tissue factor pathway inhibitor precursor 1.
HSFCAFKADDGPCKAIMKRFFFNIFTRQCEEFIYGGCEGNQNRFESLEECKKMCTRD

SEQ ID NO: 19 - protein, 57 aa, Unknown
  Kunitz-like domain of tissue factor pathway inhibitor precursor 1.
PDFCFLEEDPGICRGYITRYFYNNQTKQCERFKYGGCLGNMNNFETLEECKNICEDG

SEQ ID NO: 20 - protein, 57 aa, Unknown
  Kunitz-like domain of tissue factor pathway inhibitor precursor.
PSWCLTPADRGLCRANENRFYYNSVIGKCRPFKYSGCGGNENNFTSKQECLRACKKG

SEQ ID NO: 21 - protein, 57 aa, Unknown
  Kunitz-like domain of tissue factor pathway inhibitor precursor 2.
AEICLLPLDYGPCRALLLRYYYRYRTQSCRQFLYGGCEGNANNFYTWEACDDACWRI

SEQ ID NO: 22 - protein, 57 aa, Unknown
  Kunitz-like domain of tissue factor pathway inhibitor precursor 2.
PSFCYSPKDEGLCSANVTRYYFNPRYRTCDAFTYTGCGGNDNNFVSREDCKRACAKA

SEQ ID NO: 23 - protein, 57 aa, Unknown
  Kunitz-like domain of amyloid precursor protein homologue.
KAVCSQEAMTGPCRAVMPRTTFDLSKGKCVRFITGGCGGNRNNFESEDYCMAVCKAM

SEQ ID NO: 24 - protein, 58 aa, Unknown [sequence omitted]

SEQ ID NO: 25 - protein, 51 aa, Unknown
  Kunitz-like domain of inter-alpha-trypsin inhibitor precursor.
CQLGYSAGPCMGMTSRYFYNGTSMACETFQYGGCMGNGNNFVTEKECLQTC

SEQ ID NO: 26 - protein, 57 aa, Unknown
  Kunitz-like domain of inter-alpha-trypsin inhibitor precursor.
VAACNLPIVRGPCRAFIQLWAFDAVKGKCVLFPYGGCQGNGNKFYSEKECREYCGVP

SEQ ID NO: 27 - protein, 57 aa, Unknown
  Kunitz-like domain of amyloid precursor protein.
EVCCSEQAETGPCRAMISRWYFDVTEGKCAPFFYGGCGGNRNNFDTEEYCMAVCGSA

SEQ ID NO: 28 - protein, 51 aa, Unknown
  Kunitz-like domain of collagen alpha-3(VI) precursor.
CKLPKDEGTCRDFILKWYYDPNTKSCARFWYGGCGGNENKFGSQKECEKVC

SEQ ID NO: 29 - protein, 57 aa, Unknown
  Kunitz-like domain of HKI-B9.
PNVCAFPMEKGPCQTYMTRWFFNFETGECELFAYGGCGGNSNNFLRKEKCEKFCKFT

SEQ ID NO: 30 - DNA, 46 bp, Artificial Sequence
  5′ sense oligonucleotide used in Example 6.
gccaagcttggataaaagatatgaagaatactgcaccgccaacgca

SEQ ID NO: 31 - DNA, 35 bp, Artificial Sequence
  3′ antisense oligonucleotide used in Example 6.
ggggatcctcactgctggcggaagcagcggagcat

SEQ ID NO: 32 - DNA, 206 bp, Artificial Sequence
  Cloned bikunin cDNA fragment in Example 6.
ccaagcttggataaaagatatgaagaatactgcaccgccaacgcagtcactgggccttgccgtgcatccttcccacgctggtactttgacgtggagaggaactcctgcaataacttcatctatggaggctgccggggcaataagaacagctaccgctctgaggaggcctgcatgctccgctgcttccgccagcagtgaggatcccc

SEQ ID NO: 33 - DNA, 28 bp, Artificial Sequence
  3′ PCR primer used to amplify EST R74593.
cgaagcttcatctccgaagctccagacg

SEQ ID NO: 34 - DNA, 31 bp, Artificial Sequence
  5′ PCR primer used to amplify EST R74593.
aggatctagacaataattacctgaccaagga

SEQ ID NO: 35 - DNA, 36 bp, Artificial Sequence
  5′ PCR primer used to amplify EST R35464.
ggtctagaggccgggtcgtttctcgcctggctggga

SEQ ID NO: 36 - DNA, 19 bp, Artificial Sequence
  5′ PCR primer used to amplify EST R34808.
cacctgatcgcgagacccc

SEQ ID NO: 37 - DNA, 19 bp, Artificial Sequence
  Vector specific DNA sequencing primer (SP6).
gatttaggtgacactatag

SEQ ID NO: 38 - DNA, 20 bp, Artificial Sequence
  Vector specific DNA sequencing primer (T7).
taatacgactcactataggg

SEQ ID NO: 39 - DNA, 22 bp, Artificial Sequence
  Gene specific DNA sequencing primer.
ttacctgaccaaggaggagtgc

SEQ ID NO: 40 - DNA, 23 bp, Artificial Sequence
  Gene specific DNA sequencing primer.
aatccgctgcattcctgctggtg

SEQ ID NO: 41 - DNA, 20 bp, Artificial Sequence
  Gene specific DNA sequencing primer.
cagtcactgggccttgccgt

SEQ ID NO: 42 - DNA, 105 bp, Artificial Sequence
  5′ sense oligonucleotide used in Example 5.
gaaggggtaagcttggataaaagatatgaagaatactgcaccgccaacgcagtcactgggccttgccgtgcatccttcccacgctggtactttgacgtggagagg

SEQ ID NO: 43 - DNA, 129 bp, Artificial Sequence
  3′ antisense oligonucleotide used in Example 5.
cgcggatccctactggcggaagcagcggagcatgcaggcctcctcagagcggtagctgttcttattgccccggcagcctccatagatgaagttattgcaggagttcctctccacgtcaaagtaccagcg

SEQ ID NO: 44 - DNA, 207 bp, Artificial Sequence
  Cloned bikunin fragment in Example 5.
gaaggggtaagcttggataaaagatatgaagaatactgcaccgccaacgcagtcactgggccttgccgtgcatccttcccacgctggtactttgacgtggagaggaactcctgcaataacttcatctatggaggctgccggggcaataagaacagctaccgctctgaggaggcctgcatgctccgctgcttccgccagtagggatcc

SEQ ID NO: 45 - protein, 248 aa, Artificial Sequence
  EST derived consensus sequence of human Bikunin (Figs. 4D and 4G).
  SIGNAL (1)..(23)
MLRAEADGVSRLLGSLLLSGVLAADRERSIHDFCLVSKVVGRCRASMPRWWYNVTDGSCQLFVYGGCDGNSNNYLTKEECLKKCATVTENATGDLATSRNAADSSVPSAPRRQDSEDHSSDMFNYEEYCTANAVTGPCRASFPRWYFDVERNSCNNFIYGGCRGNKNSYRSEEACMLRCFRQQENPPLPLGSKVVVLAGLFVMVLILFLGASMVYLIRVARRNQERALRTVWSSGDDKEQLVKNTYVL

SEQ ID NO: 46 - DNA, 782 bp, Homo sapiens
acctgatcgcgagaccccaacggctggtggcgtcgcctgcgcgtctcggctgagctggccatggcgcagctgtgcgggctgaggcggagccgggcgtttctcgccctgctgggatcgctgctcctctctggggtcctggcggccgaccgagaacgcagcatccacgacttctgcctggtgtcgaaggtggtgggcagatgccgggcctccatgcctaggtggtggtacaatgtcactgacggatcctgccagctgtttgtgtatgggggctgtgacggaaacagcaataattacctgaccaaggaggagtgcctcaagaaatgtgccactgtcacagagaatgccacgggtgacctggccaccagcaggaatgcagcggattcctctgtcccaagtgctcccagaaggcaggattctgaagaccactccagcgatatgttcaactatgaagaatactgcaccgccaacgcagtcactgggccttgccgtgcatccttcccacgctggtactttgacgtggagaggaactcctgcaataacttcatctatggaggctgccggggcaataagaacagctaccgctctgaggaggcctgcatgctccgctgcttccgccagcaggagaatcctcccctgccccttggctcaaaggtggtggttctggcggggctgttcgtgatggtgttgatcctcttcctgggagcctccatggtctacctgatccgggtggcacggaggaaccaggagcgtgccctgcgcaccgtctggagcttcggagatga

SEQ ID NO: 47 - protein, 240 aa, Homo sapiens
  SIGNAL (1)..(27)
MAQLCGLRRSRAFLALLGSLLLSGVLAADRERSIHDFCLVSKVVGRCRASMPRWWYNVTDGSCQLFVYGGCDGNSNNYLTKEECLKKCATVTENATGDLATSRNAADSSVPSAPRRQDSEDHSSDMFNYEEYCTANAVTGPCRASFPRWYFDVERNSCNNFIYGGCRGNKNSYRSEEACMLRCFRQQENPPLPLGSKVVVLAGLFVMVLILFLGASMVYLIRVARRNQERALRTVWSFGD

SEQ ID NO: 48 - DNA, 1544 bp, Homo sapiens
  misc_feature (1358)..(1358): “n” is any nucleotide.
gcacgagttgggaggtgtagcgcggctctgaacgcgctgagggccgttgagtgtcgcaggcggcgagggcgcgagtgaggagcagacccaggcatcgcgcgccgagaaggccgggcgtccccacactgaaggtccggaaaggcgacttccgggggctttggcacctggcggaccctcccggagcgtcggcacctgaacgcgaggcgctccattgcgcgtgcgcgttgaggggcttcccgcacctgatcgcgagaccccaacggctggtggcgtcgcctgcgcgtctcggctgagctggccatggcgcagctgtgcgggctgaggcggagccgggcgtttctcgccctgctgggatcgctgctcctctctggggtcctggcggccgaccgagaacgcagcatccacgacttctgcctggtgtcgaaggtggtgggcagatgccgggcctccatgcctaggtggtggtacaatgtcactgacggatcctgccagctgtttgtgtatgggggctgtgacggaaacagcaataattacctgaccaaggaggagtgcctcaagaaatgtgccactgtcacagagaatgccacgggtgacctggccaccagcaggaatgcagcggattcctctgtcccaagtgctcccagaaggcaggattctgaagaccactccagcgatatgttcaactatgaagaatactgcaccgccaacgcagtcactgggccttgccgtgcatccttcccacgctggtactttgacgtggagaggaactcctgcaataacttcatctatggaggctgccggggcaataagaacagctaccgctctgaggaggcctgcatgctccgctgcttccgccagcaggagaatcctcccctgccccttggctcaaaggtggtggttctggcggggctgttcgtgatggtgttgatcctcttcctgggagcctccatggtctacctgatccgggtggcacggaggaaccaggagcgtgccctgcgcaccgtctggagctccggagatgacaaggagcagctggtgaagaacacatatgtcctgtgaccgccctgtcgccaagaggactggggaagggaggggagactatgtgtgagctttttttaaatagagggattgactcggatttgagtgatcattagggctgaggtctgtttctctgggaggtaggacggctgcttcctggtctggcagggatgggtttgctttggaaatcctctaggaggctcctcctcgcatggcctgcagtctggcagcagccccgagttgtttcctcgctgatcgatttctttcctccaggtagagttttctttgcttatgttgaattccattgcctccttttctcnatcacagaagtgatgttggaatcgtttcttttgtttgtctgatttatggtttttttaagtataaacaaaagttttttattagcattctgaaagaaggaaagtaaaatgtacaagtttaataaaaaggggccttcccctttagaataaatttccagcatgttgctttcaaaaaaaaaaaaaaaaaaa

SEQ ID NO: 49 - protein, 252 aa, Homo sapiens
  SIGNAL (1)..(27)
MAQLCGLRRSRAFLALLGSLLLSGVLAADRERSIHDFCLVSKVVGRCRASMPRWWYNVTDGSCQLFVYGGCDGNSNNYLTKEECLKKCATVTENATGDLATSRNAADSSVPSAPRRQDSEDHSSDMFNYEEYCTANAVTGPCRASFPRWYFDVERNSCNNFIYGGCRGNKNSYRSEEACMLRCFRQQENPPLPLGSKVVVLAGLFVMVLILFLGASMVYLIRVARRNQERALRTVWSSGDDKEQLVKNTYVL

SEQ ID NO: 50 - protein, 146 aa, Homo sapiens
CLVSKVVGRCRASMPRWWYNVTDGSCQLFVYGGCDGNSNNYLTKEECLKKCATVTENATGDLATSRNAADSSVPSAPRRQDSEDHSSDMFNYEEYCTANAVTGPCRASFPRWYFDVERNSCNNFIYGGCRGNKNSYRSEEACMLRC

SEQ ID NO: 51 - DNA, 1530 bp, Artificial Sequence
  Consensus bikunin sequence of Fig. 4C.
  misc_feature (46)..(46): “n” is any nucleotide.
  misc_feature (117)..(117): “n” is any nucleotide.
  misc_feature (313)..(313): “n” is any nucleotide.
gcgacctccgcgcgttgggaggtgtagcgcggctctgaacgcgtgnagggccgttgagtgtcgcaggcggcgagggcgcgagtgaggagcagacccaggcatcgcgcgccgagaagncgggcgtccccacactgaaggtccggaaaggcgacttccgggggctttggcacctggcggaccctcccggagcgtcggcacctgaacgcgaggcgctccattgcgcgtgcgtttgaggggcttcccgcacctgatcgcgagaccccaacggctggtggcgtcgctgcgcgtctcggctgagctggccatggcgcantgttgcgggctgaggcggacggcgtttctcgcctgctgggatcgctgctcctctctggggtcctggcggccgaccgagaacgcagcatccacgacttctgcctggtgtcgaaggtggtgggcagatgccgggcctccatgcctaggtggtggtacaatgtcactgacggatcctgccagctgtttgtgtatgggggctgtgacggaaacagcaataattacctgaccaaggaggagtgcctcaagaaatgtgccactgtcacagagaatgccacgggtgacctggccaccagcaggaatgcagcggattcctctgtcccaagtgctcccagaaggcaggattctgaagaccactccagcgatatgttcaactatgaagaatactgcaccgccaacgcagtcactgggccttgccgtgcatccttcccacgctggtactttgacgtggagaggaactcctgcaataacttcatctatggaggctgccggggcaataagaacagctaccgctctgaggaggcctgcatgctccgctgcttccgccagcaggagaatcctcccctgccccttggctcaaaggtggtggttctggcggggctgttcgtgatggtgttgatcctcttcctgggagcctccatggtctacctgatccgggtggcacggaggaaccaggagcgtgccctgcgcaccgtctggagctccggagatgacaaggagcagctggtgaagaacacatatgtcctgtgaccgccctgtcgccaagaggactggggaagggaggggagactatgtgtgagctttttttaaatagagggattgactcggatttgagtgatcattagggctgaggtctgtttctctgggaggtaggacggctgcttcctggtctggcagggatgggtttgctttggaaatcctctaggaggctcctcctcgcatggcctgcagtctggcagcagccccgagttgtttcctcgctgatcgatttctttcctccaggtagagttttctttgcttatgttgaattccattgcctcttttctcatcacagaagtgatgttggaatcgtttcttttgtttgtctgatttatggtttttttaagtataaacaaaagttttttattagcattctgaaagaaggaaagtaaaatgtacaagtttaataaaaaggggccttcccctttagaataaaaaaaaaaaaaaaaaaaaaaaaaaaaa

SEQ ID NO: 52 - protein, 170 aa, Homo sapiens
ADRERSIHDFCLVSKVVGRCRASMPRWWYNVTDGSCQLFVYGGCDGNSNNYLTKEECLKKCATVTENATGDLATSRNAADSSVPSAPRRQDSEDHSSDMFNYEEYCTANAVTGPCRASFPRWYFDVERNSCNNFIYGGCRGNKNSYRSEEACMLRCFRQQENPPLPLGSK

SEQ ID NO: 53 - protein, 27 aa, Homo sapiens
MAQLCGLRRSRAFLALLGSLLLSGVLA

SEQ ID NO: 54 - protein, 23 aa, Homo sapiens
MLRAEADGVSRLLGSLLLSGVLA

SEQ ID NO: 55 - DNA, 102 bp, Artificial Sequence
  5′ sense oligonucleotide used for construct #2 in Example 5.
gaaggggtaagcttggataaaagagaagaatactgtactgctaatgctgttactggtccatgtagagcttcttttccaagatggtactttgatgttgaaaga

SEQ ID NO: 56 - DNA, 129 bp, Artificial Sequence
  3′ antisense oligonucleotide used for construct #2 in Example 5.
actggatcctcattggcgaaaacatctcaacatacaggcttcttcagatctgtaagaatttttattacctctacaaccaccgtaaataaaattattacaagaatttctttcaacatcaaagtaccatct

SEQ ID NO: 57 - DNA, 108 bp, Artificial Sequence
  5′ sense oligonucleotide used for construct #3 in Example 5.
gaaggggtaagcttggataaaagaaattacgaagaatactgtactgctaatgctgttactggtccatgtagagcttcttttccaagatggtactttgatgttgaaaga

SEQ ID NO: 58 - DNA, 117 bp, Artificial Sequence
  5′ sense oligonucleotide used for construct #4 in Example 5.
gaaggggtaagcttggataaaagagatatgtttaattacgaagaatactgtactgctaatgctgttactggtccatgtagagcttcttttccaagatggtactttgatgttgaaaga

SEQ ID NO: 59 - DNA, 19 bp, Artificial Sequence
  Sense oligonucleotide used in PCR in Example 8.
cacctgatcgcgagacccc

SEQ ID NO: 60 - DNA, 23 bp, Artificial Sequence
  Antisense oligonucleotide used in PCR in Example 8.
ctggcggaagcagcggagcatgc

SEQ ID NO: 61 - DNA, 45 bp, Artificial Sequence
  Oligonucleotide used in in vitro mutagenesis in Example 9.
cgcgtctcggctgacctggccctgcagatggcgcacgtgtgcggg

SEQ ID NO: 62 - DNA, 60 bp, Artificial Sequence
  Oligonucleotide used in in vitro mutagenesis in Example 9.
ctgccccttggctcaaagtaggaagatcttccccccgggggggtggttctggcggggctg

SEQ ID NO: 63 - protein, 14 aa, Homo sapiens
LRCFRQQENPPPLG

SEQ ID NO: 64 - protein, 20 aa, Homo sapiens
ADRERSIHDFCLVSKVVGRC

SEQ ID NO: 65 - protein, 20 aa, Homo sapiens
FNYEEYCTANAVTGPCRASF

SEQ ID NO: 66 - protein, 11 aa, Homo sapiens
PRYVDGSQFYG

SEQ ID NO: 67 - protein, 55 aa, Homo sapiens
VVVLAGLFVMVLILFLGASMVYLIRVARRNQERALRTVWSSGDDKEQLVKNTYVL

SEQ ID NO: 68 - protein, 43 aa, Homo sapiens
VVVLAGLFVMVLILFLGASMVYLIRVARRNQERALRTVWSFGD

SEQ ID NO: 69 - protein, 55 aa, Homo sapiens; identical to SEQ ID NO: 67

SEQ ID NO: 70 - protein, 213 aa, Homo sapiens
ADRERSIHDFCLVSKVVGRCRASMPRWWYNVTDGSCQLFVYGGCDGNSNNYLTKEECLKKCATVTENATGDLATSRNAADSSVPSAPRRQDSEDHSSDMFNYEEYCTANAVTGPCRASFPRWYFDVERNSCNNFIYGGCRGNKNSYRSEEACMLRCFRQQENPPLPLGSKVVVLAGLFVMVLILFLGASMVYLIRVARRNQERALRTVWSFGD

SEQ ID NO: 71 - protein, 225 aa, Homo sapiens
ADRERSIHDFCLVSKVVGRCRASMPRWWYNVTDGSCQLFVYGGCDGNSNNYLTKEECLKKCATVTENATGDLATSRNAADSSVPSAPRRQDSEDHSSDMFNYEEYCTANAVTGPCRASFPRWYFDVERNSCNNFIYGGCRGNKNSYRSEEACMLRCFRQQENPPLPLGSKVVVLAGLFVMVLILFLGASMVYLIRVARRNQERALRTVWSSGDDKEQLVKNTYVL

SEQ ID NO: 72 - protein, 19 aa, Homo sapiens
  MISC_FEATURE (9)..(9): “Xaa” is Ile, Thr, Asn, or Ser.
  MISC_FEATURE (11)..(11): “Xaa” is Val, Ala, Glu, or Gly.
  MISC_FEATURE (17)..(17): “Xaa” is Ser, Pro, Thr, or Ala.
  MISC_FEATURE (19)..(19): “Xaa” is Tyr, His, Asn, or Asp.
RPLQRYVSXIXRIIAPXTX

SEQ ID NO: 73 - protein, 108 aa, Homo sapiens
PGHQQECSGFLCPKSPRRQDSEDHSSDMFNYEEYCTANAVTGPCRASFPRWYFDVERNSCNNFIYGGCRGNKNSYRSEEACMLRCFRQQENPPLPLGSKVVVLAGAVS

SEQ ID NO: 74 - protein, 31 aa, Homo sapiens
  MISC_FEATURE (25)..(25): “Xaa” is Asp or Glu.
SFSWGASMVLLIPGGKEEPGACPAXRLELRR

SEQ ID NO: 75 - DNA, 511 bp, Artificial Sequence
  Corrected version of EST R74593 (Fig. 3).
  misc_feature (425)..(425): “n” is any nucleotide.
  misc_feature (482)..(482): “n” is any nucleotide.
  misc_feature (510)..(510): “n” is any nucleotide.
gcaataattacctgaccaaggaggagtgcctcaagaaatgtgccactgtcacagagaatgccacgggtgacctggccaccagcaggaatgcagcggattcctctgtcccaagtgctcccagaaggcaggattctgaagaccactccagcgatatgttcaactatgaagaatactgcaccgccaacgcagtcactgggccttgccgtgcatccttcccacgctggtactttgacgtggagaggaactcctgcaataacttcatctatggaggctgccggggcaataagaacagctaccgctctgaggaggcctgcatgctccgctgcttccgccagcaggagaatcctcccctgccccttggctcaaaggtggtggttctggccggggctgtttcgtgatggtgttgatccttttcctggggagcntccatggtcttactgattccgggtggcaaggaggaaccaggagcgtgccctgcggancgtctggagcttcggagatgacaagggnt

SEQ ID NO: 76 - protein, 31 aa, Artificial Sequence; identical to SEQ ID NO: 74
  Amino acids 184-214 of the translation of the consensus DNA sequence in Fig. 3.
  MISC_FEATURE (25)..(25): “Xaa” is Asp or Glu.

SEQ ID NO: 77 - DNA, 312 bp, Homo sapiens
  misc_feature (45)..(45): “n” is any nucleotide.
  misc_feature (49)..(49): “n” is any nucleotide.
  misc_feature (118)..(118): “n” is any nucleotide.
  misc_feature (231)..(231): “n” is any nucleotide.
  misc_feature (305)..(305): “n” is any nucleotide.
gcgacctccgcgcgttgggaggtgtagcgcggctctgaacgcgtngagnggccgttgagtgtcgcaggcggcgagggcgcgagtgaggagcagacccaggcatcgcgcgccgagaagncgggcgtccccacactgaaggtccggaaaggcgacttccgggggctttggcacctggcggaccctcccggagcgtcggcacctgaacgcgaggcgctccattgcgcgtgcgtntgaggggcttcccgcacctgatcgcgagaccccaacggctggtggcgtcgcctgcgcgtctcggctgagctggncatgtcg

SEQ ID NO: 78 - DNA, 330 bp, Homo sapiens
  misc_feature (117)..(117): “n” is any nucleotide.
  misc_feature (123)..(123): “n” is any nucleotide.
  misc_feature (321)..(321): “n” is any nucleotide.
gcgacctccgcgcgttgggaggtgtagcgcggctctgaacgcgtgcagggccgttgagtgtcgcaggcggcgagggcgcgagtgaggagcagacccaggcatcgcgcgccgagaagncgggcntccccacactgaaggtccggaaaggcgacttccgggggctttggcacctggcggaccctcccggagcgtggcacctgaacgcgaggcgctccattgcgcgtgcgtttgaggggcttcccgcacctgatcgcgagaccccaacggctggtggcgtcgcctgcgcgtctcggctgagctggccatggcgcactgtgcggngctgaggcg

SEQ ID NO: 79 - DNA, 283 bp, Homo sapiens
  misc_feature (9)..(9): “n” is any nucleotide.
  misc_feature (11)..(11): “n” is any nucleotide.
  misc_feature (222)..(222): “n” is any nucleotide.
  misc_feature (231)..(231): “n” is any nucleotide.
  misc_feature (262)..(262): “n” is any nucleotide.
  misc_feature (267)..(267): “n” is any nucleotide.
  misc_feature (274)..(274): “n” is any nucleotide.
ttgagtgtngnaggcggcgagggcgcgagtgaggagcagacccaggcatcgcgcgccgagaaggccgggcgtccccacactgaaggtccggaaaggcgacttccgggggctttggcacctggcggaccctcccggagcgtcggcacctgaacgcgaggcgctccattgcgcgtgcgtttgaggggcttcccgcacctgatcgcgagaccccaacggctggtngcgtcgctncgcgtctcggctgagcttggccatggcgcantgttncgggctnaggcggacg

SEQ ID NO: 80 - DNA, 423 bp, Homo sapiens
  misc_feature (44)..(44): “n” is any nucleotide.
  misc_feature (46)..(46): “n” is any nucleotide.
  misc_feature (76)..(76): “n” is any nucleotide.
  misc_feature (114)..(114): “n” is any nucleotide.
  misc_feature (187)..(187): “n” is any nucleotide.
  misc_feature (268)..(268): “n” is any nucleotide.
  misc_feature (309)..(309): “n” is any nucleotide.
  misc_feature (317)..(317): “n” is any nucleotide.
  misc_feature (332)..(332): “n” is any nucleotide.
  misc_feature (370)..(370): “n” is any nucleotide.
ggcgacctccgcgcgttgggaggtgtagcgcgctctgaacgggnangggccgttgagtgtcgcaggcggcagggcngagtgaggagcagacccaggcatcgcgcgccgagaagncgggcgtccccacactgaaggtccggaaaggcgacttccgggggctttggcacctggcggacgtcccggagcnggcacctgaacgcgaggcgctccattgcgcgtgcgtttgaggggcttcccgcacctgatcgcgagaccccaacggctggtngcgtcgctggcgcgttctcggctgagctggccatggcgcantgttgcgngctgaggcggaccgncgtttttcttcgccttgctgggattcgcttgcttcctntctgggggttcctgggcggccgaccgagaacgcagcatccaagaatttttgcc

SEQ ID NO: 81 - DNA, 344 bp, Homo sapiens
  misc_feature (35)..(35): “n” is any nucleotide.
  misc_feature (148)..(148): “n” is any nucleotide.
  misc_feature (235)..(235): “n” is any nucleotide.
  misc_feature (261)..(261): “n” is any nucleotide.
  misc_feature (272)..(272): “n” is any nucleotide.
  misc_feature (293)..(293): “n” is any nucleotide.
  misc_feature (300)..(300): “n” is any nucleotide.
  misc_feature (313)..(313): “n” is any nucleotide.
  misc_feature (320)..(320): “n” is any nucleotide.
ggaggagcagacccaggcatcgcgcgccgagaagncgggcgtccccacactgaaggtccggaaaggcgacttccgggggctttggcacctggcggaccctcccggagcgtcggcacctgaacgcgaggcgctccattgcgcgtgcgtntggaggggcttcccgcacctgatcgcgagaccccaacggctggtgggcgtcgctgcgcgtcttcggctgagctgggccatggcgcanttgttgcgggctgaggcggacgcggncgttttttcgnccttgctgggattcgttgttnctctctnggggttctggggnggccgancgagaacgcaagcattcacgattt

SEQ ID NO: 82 - DNA, 253 bp, Homo sapiens
  misc_feature (56)..(56): “n” is any nucleotide.
  misc_feature (137)..(137): “n” is any nucleotide.
  misc_feature (145)..(145): “n” is any nucleotide.
  misc_feature (159)..(159): “n” is any nucleotide.
  misc_feature (233)..(233): “n” is any nucleotide.
ggaccctcccggagcgtcggcacctgaacgcgaggcctccattgcggtgcgtgtgnaggggcttcccgcacctgatcgcgagaccccaacggctggtggcgtcgctgcgcgtctcggctgagctggccatggcgcantgttgcgngctgaggcggcggncgttttctcgcctgctgggatcgctgctcctctctggggtcctggcggccgaccgagaacgcagcatccacganttcttcctggtgttcgaagg

SEQ ID NO: 83 - DNA, 419 bp, Homo sapiens
  misc_feature (20)..(20): “n” is any nucleotide.
  misc_feature (26)..(26): “n” is any nucleotide.
  misc_feature (95)..(95): “n” is any nucleotide.
  misc_feature (292)..(292): “n” is any nucleotide.
  misc_feature (313)..(315): “n” is any nucleotide.
ttagcgcggctctgaacgcnagaagnggccgttgagtgtcgcaggcggcgagggcgcgagtgaggagcagacccaggcatcgcgcgccgagaagncgggcgtccccacactgaaggtccggaaaggcgacttccgggggctttggcacctggcggaccctcccggagcgtcggcacctgaacgcgaggcgctccattgcgcgtgcgtttgaggggcttcccgcacctgatcgcgagaccccaacggctggtggcgtcgcctgcgcgtctcggctgagctggccatggcgcantggtgcgggcttgaggcggannngccgtttctcgcctgctgggatcgctgctcctctctggggtcctggcggccgaccgagaacgcagcatccacgacttctgcctggtgtcgaaggtggtgggcag

SEQ ID NO: 84 - DNA, 477 bp, Homo sapiens
  misc_feature (27)..(27): “n” is any nucleotide.
  misc_feature (139)..(139): “n” is any nucleotide.
  misc_feature (223)..(223): “n” is any nucleotide.
  misc_feature (232)..(232): “n” is any nucleotide.
  misc_feature (302)..(302): “n” is any nucleotide.
  misc_feature (310)..(310): “n” is any nucleotide.
  misc_feature (322)..(322): “n” is any nucleotide.
  misc_feature (328)..(328): “n” is any nucleotide.
  misc_feature (357)..(357): “n” is any nucleotide.
  misc_feature (375)..(375): “n” is any nucleotide.
  misc_feature (392)..(392): “n” is any nucleotide.
  misc_feature (398)..(398): “n” is any nucleotide.
  misc_feature (405)..(405): “n” is any nucleotide.
  misc_feature (427)..(427): “n” is any nucleotide.
  misc_feature (437)..(437): “n” is any nucleotide.
  misc_feature (449)..(449): “n” is any nucleotide.
  misc_feature (458)..(458): “n” is any nucleotide.
  misc_feature (474)..(474): “n” is any nucleotide.
agacccaggcatcgcgcgccgagaagncgggcgtccccacactgaaggtccggaaaggcgacttccgggggctttggcacctggcggaccctcccggagcgtcggcacctgaacgcgaggcctccattgccgtgcgttngaggggcttcccggaacttgatcgcgagaccccaacggctggtggcgtcgctgcgcgtcctcggctgagctggccatggcgcantggtgccgngctgaggccggagggccggtttctcgccttgctgggatcgctgctcctctctggggtcctggcggccgancgaagaangcagcaatccangaattnctgcctggtgttcgaaagttggtgggcanattccggggccttcatgnctaaggttggttggtanaatgtnaattaangattcttgcaactgtttgtgtnattggggctnttaaacggaaanacaataatnacctgaccaaagaagnaat

SEQ ID NO: 85 - DNA, 393 bp, Homo sapiens; identical to SEQ ID NO: 12
  misc_feature (361)..(361): “n” is any nucleotide.
  misc_feature (367)..(367): “n” is any nucleotide.
  misc_feature (384)..(384): “n” is any nucleotide.
  misc_feature (390)..(390): “n” is any nucleotide.

SEQ ID NO: 86 - DNA, 428 bp, Homo sapiens
  misc_feature (3)..(3): “n” is any nucleotide.
  misc_feature (11)..(12): “n” is any nucleotide.
  misc_feature (17)..(17): “n” is any nucleotide.
  misc_feature (48)..(48): “n” is any nucleotide.
  misc_feature (425)..(425): “n” is any nucleotide.
gcngcgcgttnntcgcntgctgggatcgctgcacctctctggggtcgnggcggccgaccgagaacgcagcatccacgacttctgcctggtgtcgaaggtggtgggcagatgccgggcctccatgcctaggtggtggtacaatgtcactgacggatcctgccagctgtttgtgtatgggggctgtgacggaaacagcaataattacctgaccaaggaggagtgcctcaagaaatgtgccactgtcacagagaatgccacgggtgacctggccaccagcaggaatgcagcggattcctctgtcccaagtgctcccagaaggcaggattctgaagaccactccagcgatatgttcaactatgaagaatactggcaccgccaacgcattcactgggcctgcgtgcatccttcccacgctggtactttgncgt

SEQ ID NO: 87 - DNA, 425 bp, Homo sapiens
  misc_feature (7)..(7): “n” is any nucleotide.
  misc_feature (403)..(403): “n” is any nucleotide.
  misc_feature (409)..(409): “n” is any nucleotide.
ctgggantcgctgctcctctctggggtcctggcggccgaccgagaacgcagcatccacgacttctgcctggtgtcgaaggtggtgggcagatgccgggcctccatgcctaggtggtggtacaatgtcactgacggatcctgccagctgtttgtgtatgggggctgtgacggaaacagcaataattacctgaccaaggaggagtgcctcaagaaatgtgccactgtcacagagaatgccacgggtgacctggccaccagcaggaatgcagcggattcctctgtcccaagtgctcccagaaggcaggattctgaagaccactccagcgatatgttcaactatgaagaatactgcaccgccaacgcagtcactggggccttgcgtggaatcctttcccacgctggnaatttngacgttgagaaggaac

SEQ ID NO: 88 - DNA, 343 bp, Homo sapiens
  misc_feature (48)..(48): “n” is any nucleotide.
  misc_feature (62)..(62): “n” is any nucleotide.
  misc_feature (211)..(211): “n” is any nucleotide.
  misc_feature (232)..(232): “n” is any nucleotide.
  misc_feature (245)..(245): “n” is any nucleotide.
  misc_feature (309)..(309): “n” is any nucleotide.
  misc_feature (318)..(318): “n” is any nucleotide.
gattcggcacaggggaaacagcaataattacctgaccaaggaggagtncctcaagaaatgtnccactgtcacagagaatgccacgggtgacctggccaccagcaggaatgcagcggattcctctgtcccaagtgctcccagaaggcaggattctgaagaccactccagcgatatgttcaactatgaagaatactgcaccgccaacgcagtncactgggccttgcgtggcatnccttcccacgctngtactttgacgtggagaggaactcctggcaataacttcatctatggaggcttgccggggcaatnaagaacagnttaccgctctttaggaggcctgcat

SEQ ID NO: 89 - DNA, 510 bp, Homo sapiens; identical to SEQ ID NO: 14
  misc_feature (424)..(424): “n” is any nucleotide.
  misc_feature (481)..(481): “n” is any nucleotide.
  misc_feature (509)..(509): “n” is any nucleotide.

SEQ ID NO: 90 - DNA, 293 bp, Homo sapiens
  misc_feature (257)..(257): “n” is any nucleotide.
gctaccgctctgaggaggcctgcatgctccgctgcttccgccagcaggagaatcctcccctgccccttggctcaaaggtggtggttctggcggggctgttcgtgatggtgttgatcctcttcctggggagcctccatggtctacctgatccgggtggcacggagggaaccagggagcgtgccctgcgcaccgtctgggagctccggagatgacaagggagcagctgggtgaagaacacatatgttcctgttgaccgncctgttcgccaagaggattgggggaagggaggggga

SEQ ID NO: 91 - DNA, 282 bp, Homo sapiens
  misc_feature (19)..(19): “n” is any nucleotide.
  misc_feature (147)..(147): “n” is any nucleotide.
ttccgccaagcaggaaaantcctcccctcccccttggctcaaaggtggtggttcctggcggggctgttcgtgatggtgttgatccctccttcccgggagcctcccatggtcctaccctgatccgggtggcacggaggaacccaggancgtgccctgcgcaccgtctggagctccggagatgacaaggagcagctggtgaagaacacatatgtcctgtgaccgccctgtcgccaagaggactggggaagggaggggagactatgtgtgagctttttttaaata

SEQ ID NO: 92 - DNA, 390 bp, Homo sapiens
  misc_feature (33)..(33): “n” is any nucleotide.
  misc_feature (55)..(55): “n” is any nucleotide.
  misc_feature (118)..(118): “n” is any nucleotide.
  misc_feature (213)..(213): “n” is any nucleotide.
  misc_feature (228)..(228): “n” is any nucleotide.
  misc_feature (259)..(259): “n” is any nucleotide.
  misc_feature (267)..(267): “n” is any nucleotide.
  misc_feature (324)..(324): “n” is any nucleotide.
  misc_feature (333)..(333): “n” is any nucleotide.
  misc_feature (344)..(344): “n” is any nucleotide.
  misc_feature (387)..(387): “n” is any nucleotide.
gagaggaactcctgcaataacttcatctatggnggctgccggggaataagaacanctaccgctctgaggaggcctgcgtgctccgctgcttccgctgtgtgttctcttccaggccagcaggagaatcctcccctgccccttggctcaaaggtggtggttctggcggggctgttcgtgatggtgttgatcctcttcctgggagcctccatggtntacctgatccgggtngcacggaggaaccagggagcgtgccctgcgnaccgtctnggagctccggagatgacaaggagcagctggtgaagaacacatatgtcctgtgaccgncctgttcgncaagaggactnggggaaaggggaggggagattatgtgttgagttttttttaaantag

SEQ ID NO: 93 - DNA, 406 bp, Homo sapiens
  misc_feature (306)..(306): “n” is any nucleotide.
  misc_feature (328)..(328): “n” is any nucleotide.
  misc_feature (342)..(342): “n” is any nucleotide.
  misc_feature (365)..(365): “n” is any nucleotide.
  misc_feature (370)..(370): “n” is any nucleotide.
  misc_feature (377)..(377): “n” is any nucleotide.
  misc_feature (382)..(382): “n” is any nucleotide.
  misc_feature (402)..(402): “n” is any nucleotide.
gattcggaacgaggagccggggcaataagaacagctaccgctctgaggaggcctgcatgctccgctgcttccgccagcaggagaatcctcccctgccccttggctcaaaggtggtggttctggcggggctgttcgtgatggtgttgatcctcttcctgggagcctccatggtctacctgatccgggtggcacggaggaaccagggagcgtgccctgcgcaccgtctgggagctccggagatgacaagggagcagctggtgaagaacacatatgttcctgttgaccgccctgttcgccaagagggantgggggaaggggagggggagantattgttgttgagnttttttttaaaattaggaggggnttganttcgggnttttnagttgatccatttagggggntgag

SEQ ID NO: 94 - DNA, 360 bp, Homo sapiens
  misc_feature (1)..(1)
  misc_feature (142)..(142): “n” is any nucleotide.
  misc_feature (339)..(339): “n” is any nucleotide.
  misc_feature (347)..(347): “n” is any nucleotide.
nggccttgcagtgctccgctgcttccgccagcaggagaatcctcccctgccccttggctcaaaggtggtggttctggcggggctgttcgtgatggtgttgatcctcttcctgggagcctccatggtctacctgatccgggtngcacggaggaaccaggagcgtgccctgcgcaccgtctggagctccggagatgacaaggagcagctggtgaagaacacatatgtcctgtgaccgccctgtcgccaagaggactggggaagggaggggagactatgtgtgagctttttttaaatagagggattgactcggatttgagtgatcattagggctgaggtctntttctctnggaggtaggacga

SEQ ID NO: 95 - DNA, 438 bp, Homo sapiens
  misc_feature (334)..(334): “n” is any nucleotide.
  misc_feature (368)..(368): “n” is any nucleotide.
  misc_feature (376)..(376): “n” is any nucleotide.
cggggctgttcgtgatggtgttgatcctcttcctgggagcctccatggtctacctgatccgggtggcacggaggaaccaggagcgtgccctgcgcaccgtctggagctccggagatgacaaggagcagctggtgaagaacacatatgtcctgtgaccgccctgtcgccaagaggactggggaagggaggggagactatgtgtgagctttttttaaatagagggattgactcggatttgagtgatcattagggctgaggtctgtttctctgggaggtaggacggctgcttcctgggtcttggcagggatggggtttgctttgggaaatcctcttnggaggctcctccttcgcatgggccttgcagtctnggcagcancccccgagtttttttccttcgctgatccgatttcttttcctccaggtaagaatttttctttt

SEQ ID NO: 96 - DNA, 448 bp, Homo sapiens
  misc_feature (108)..(108)
  misc_feature (261)..(261): “n” is any nucleotide.
gggaaccaggagcgtgccctgcgcaccggtctggagctccggagatgacaaggagcagctggtgaagaacacatatgtcctgtgaccgccctgtcgccaagaggactngggaagggaggggagactatgtgtgagctttttttaaatagagggattgactcggatttgagtgatcattagggctgaggtctgtttctctgggaggtaggacggctgcttcctggtctggcagggatgggtttgctttggagaatcctctangaggctcctcctcgcatggcctgcagtctggcagcagccccgagttgtttcctcgctgatcgatttctttcctccaggtagagttttctttgcttatgttgaattccattgcctcttttctcatcacagaagtgatgttggaatcgtttcttttgttttgtctgatttatgggttttttttaagtat

SEQ ID NO: 97 - DNA, 331 bp, Homo sapiens
  misc_feature (20)..(20): “n” is any nucleotide.
  misc_feature (30)..(30): “n” is any nucleotide.
attagggctgaggtctgttnctctgggagntaggacggctgccttcctggtctggcagggatgggtttgctttggaaatcctctaggaggctcctcctcgcatggcctgcagttctgcagcagccccgagttgtttcctcgctgatcgatttctttcctccaggtagagttttctttgcttatgttgaattccattgcctcttttctcatcacagaagtgatgttggaatcgtttcttttgtttgtctgatttatggtttttttaagtataaacaaaagttttttattagcattctgaaagaaggaaagtaaaatgtacaagtttaataaa

SEQ ID NO: 98 - DNA, 373 bp, Homo sapiens
  misc_feature (45)..(45)
  misc_feature (102)..(102): “n” is any nucleotide.
  misc_feature (105)..(105): “n” is any nucleotide.
  misc_feature (159)..(159): “n” is any nucleotide.
  misc_feature (174)..(174): “n” is any nucleotide.
  misc_feature (213)..(213): “n” is any nucleotide.
  misc_feature (337)..(337): “n” is any nucleotide.
gattgactcggatttgagtgatcattagggctgaggtctgtttcnctgggaggtaggacggctgctcccctggtctggcagggatgggtttgctttggaaanccnctaggaggctcctcctcgcatggcctgcagtctggcagcagccccgagttgttncctcgctgatcgatntctttcccccaggtagagttttctttgcttatgttgaantccattgcctcttttctcatcacagaagtgatgttggaatcgtttcttttgtttgtctgatttatggtttttttaagtataaacaaaagttttttattagcattctgaaagaaggaaagtaaantgtacaagtttaataaaaaggggccttcccctttaa

SEQ ID NO: 99 - DNA, 380 bp, Homo sapiens
gattgactcggatttggagtgatcattagggctgaggtctgtttctctgggaggtaggacggctgcttcctggtctggcagggatgggtttgctttggaaatcctctaggaggctcctccttcgcatggcctgcagtctggcagcagccccgagttgtttcctcgctgatcgatttctttcctccaggtagagttttctttgcttatgttgaattccattgcctcttttctcatcacagaagtgatgttggaatcgtttcttttgtttgtctgatttatggtttttttaagtataaacaaaagttttttattagcattctgaaagaaggaaagtaaaatgtacaagtttaataaaaaggggccttcccctttagaataaa

SEQ ID NO: 100 - DNA, 320 bp, Homo sapiens
  misc_feature (304)..(304): “n” is any nucleotide.
  misc_feature (309)..(309): “n” is any nucleotide.
tctggcagggatgggtttgctttggaaatcctctaggaggctcctcctcgcatggcctgcagtctggcagcagcccgagttgtttcctcgctgatcgatttctttcctccaggtagagttttctttgcttatgttgaattccattgcctcttttctcatcacagaagtgatgttggaatcgtttcttttgtttgtctgatttatggtttttttaagtataaacaaaagttttttattagcattctgaaagaaggaaagtaaaatgtacaagtttaataaaaaggggccttcccctttaggaatnaaaanaaaaaagggtg

SEQ ID NO: 101 - DNA, 397 bp, Homo sapiens
  misc_feature (24)..(24): “n” is any nucleotide.
gattgactcggatttgagtgatcnattagggctgaggtctgtttctctgggaggtaggacggctgcttcatggtctggcagggatgggtttgctttggaaatcctctaggaggctcctcctcgcatggcctgcagtctgcagcagccccgagttgtttcctcgctgatcgatttctttcctccaggtagagttttctttgcttatgttgaattccattgcctcttttctcatcacagaagtgatgttggaatcgtttcttttgtttgtctgatttatggtttttttaagtataaacaaaagttttttattagcattctgaaagaaggaaagtaaaatgtacaagtttaataaaaaggggccttcccctttagaataaatttcagcatgtgctttcaa

SEQ ID NO: 102 - DNA, 289 bp, Homo sapiens
  misc_feature (61)..(61): “n” is any nucleotide.
  misc_feature (74)..(74): “n” is any nucleotide.
  misc_feature (122)..(122): “n” is any nucleotide.
  misc_feature (184)..(184): “n” is any nucleotide.
gaggctcctcctcgcatggcctgcagtcttggcagcagccccgagttgtttcctcgctgancgatttctttccnccaggtagagttttctttgcttatgttgaattccattgcctcttttcncatcacagaagtgatgttggaatcgtttcttttgtttgtctgatttatggtttttttaagtntaaacaaaagttttttattagcattctgaaagaaggaaagtaaaatgtacaagtttaataaaaaggggccttcccctttagaataaaaaaaaaaaaaaaaaaaaa

SEQ ID NO: 103 - DNA, 311 bp, Homo sapiens
  misc_feature (7)..(7): “n” is any nucleotide.
cttttgnaaatcctctaggaggctcctcctcgcatggcctgcagtctgcagcagccccgagttgtttcctcgctgatcggatttctttcctccaggtagagttttctttgcttatgttgaattccattgcctcttttctcatcacagaagtgatgttggaatcgtttcttttgtttgtctgatttatggtttttttaagtataaacaaaagttttttattagcattctgaaagaaggaaagtaaaatgtacaagtttaataaaaaggggccttcccctttagaataaatttcagcatgtgctttcaaaaaa

SEQ ID NO: 104 - DNA, 338 bp, Homo sapiens
  misc_feature (32)..(32): “n” is any nucleotide.
  misc_feature (67)..(67): “n” is any nucleotide.
  misc_feature (136)..(136): “n” is any nucleotide.
ggtctggcagggatgggtttgcctttggaaancctctaggaggctcctcctcgcatggcctgcagtnctggcagcagaccccgagttgtttcctcgctgatcgatttctttacccccaggtagagttttcctttgncttatgttgaattccattgcctcttttactcatcacagaagtgatgttggaatcgtttcttttgtttgtctgatttatggtttttttaagtataaacaaaagttttttattagcattctgaaagaaggaaagtaaaatgtacaagtttaataaaaaggggccttcccctttagaataaaaaaaaaaaaaaaaaaaaaaaaaa

SEQ ID NO: 105 - DNA, 343 bp, Homo sapiens
  misc_feature (13)..(13): “n” is any nucleotide.
  misc_feature (19)..(19): “n” is any nucleotide.
  misc_feature (107)..(107): “n” is any nucleotide.
ccctgggtcctgncaaggnatggggtttgctttggaaatcctcttaggaggctcctcctcgcatggcctgcagtctggcagcagccccgagttgtttcctcgctgancgatttctttcctccaggtagagttttctttgcttatgttgaattccattgcctcttttctcatcacagaagtgatgttggaatcgtttcttttgtttgtctgatttatggtttttttaagtataaacaaaagttttttattagcattctgaaagaaggaaagtaaaatgtacaagtttaataaaaaggggccttcccctttagaataaaaaaaaaaaaaaaaaaaaaaaaaaaaa

SEQ ID NO: 106 - protein, 4 aa, Homo sapiens
LGSK

(19) United States
(12) Patent Application Publication
(10) Pub. No.: US 2007/0086956 A1
(43) Pub. Date: Apr. 19, 2007

(54) Method for accelerating the rate of mucociliary clearance

(75) Inventors: Roderick L. Hall, Woodham (GB); Christopher T. Poll, Windsor Meadows (GB); Benjamin B. Newton, Reading (GB); William J.A. Taylor, Windsor (GB)

Correspondence Address: Lisa A. Haile, J.D., Ph.D.;DLA PIPER US LLP, Sute 1100, 4365 Executive Drive, San Diego, CA, 92121-2133, US

(73) Assignee: BAYER AKTIENGESELLSCHAFT

(21) Appl. No.: 11/542,828

(22) Filed: Oct. 2, 2006

Related U.S. Application Data
(63) Continuation of application No. 09/441,966, filed on Nov. 17, 1999.
Application No. 09/441,966 is a continuation-in-part of application No. 09/218,913, filed on Dec. 22, 1998, now abandoned.

(51) Int. Cl. A61K 38/17; A61K 9/14

(52) U.S. Cl. 424/46; 514/8

(57) ABSTRACT

The instant invention provides for a composition and method for using Kunitz-type serine protease inhibitors, e.g., aprotinin or bikunin, for stimulating the rate of mucociliary clearance of mucus and sputum in lung airways of subjects afflicted with mucociliary dysfunctions such as cystic fibrosis.

### CROSS REFERENCE

Under 35 USC § 120, this application is a continuation application of U.S. application Ser. No. 09/441,966 filed Nov. 17, 1999, now pending, which is a continuation-in-part of U.S. patent application Ser. No. 09/218,913, filed Dec. 22, 1998.

### FIELD OF THE INVENTION

The present invention relates to compositions comprising serine protease inhibitor proteins which stimulate the rate of mucociliary clearance of mucus and sputum in lung airways. The present invention also relates to methods for stimulating the rate of mucociliary clearance in mammals.

### BACKGROUND OF THE INVENTION

Problem Addressed

Mucociliary dysfunction, characterized by the inability of ciliated epithelium to clear mucus and sputum in lung airways, is a serious complication of chronic obstructive lung diseases such as Chronic Bronchitis (CB), Bronchiectasis (BE), asthma and, especially, Cystic Fibrosis (CF). Patients suffering from mucociliary dysfunction are particularly vulnerable to secondary bacterial infections. Treatment and maintenance modalities for CF and other respiratory diseases associated with mucociliary dysfunction and the need for improved treatments have been described. See, for instance, Braga “Drugs in Bronchial Mucology, Raven Press, New York, 1989; Lethem et al, Am Rev. Respir. Dis. 142: 1053-1058, 1990; U.S. Pat. No. 5,830,436.

Cystic Fibrosis

Cystic fibrosis (CF) is an autosomal recessive disease that causes abnormalities in fluid and electrolyte transport in exocrine epithelia. Mutations within the DNA coding for a protein termed the cystic fibrosis transmembrane conductance regulator (CFTR) have been found in virtually all CF patients. Cells of the lung are particularly affected. Di Santagrese et al, Am J. Med. 66: 121-132 (1979).

In CF, the luminal border of the airway mucosal cell is unresponsive to cAMP-dependant protein kinase activation of membrane chloride ion channels. The cell permeability to Cl⁻ is impaired and Na⁺ absorption across the cell membrane is accelerated. Both of these electrolyte imbalances tend to reduce the level of hydration of the airway mucus thus contributing to the viscous lung secretions characteristic of CF. Knowles, Clin. Chest. Med. 11: 75 (1986). Adventitious bacteria and mycoplasmas enter the lung airways and establish colonies within the mucus. The thick mucus associated with CF isolates these pathogens from the immune system. Since mucociliary clearance is reduced in CF patients, bacterial clearance is also reduced. Lung congestion and infection are thus common. The prolonged presence of these pathogenic agents invariably initiates inflammatory reactions that compromise lung function. Bedrossian et al., Human Pathol. 7: 195-204, 1976.

Mucus viscosity in CF lungs is in part due to the decreased hydration of the mucus as related to Cl— channel malfunction and modification of sodium (Na˜) ion concentration in the airway surface liquid (ASL) that change the rate of airway mucociliary clearance (MCC). The mechanisms involved in mucus transport have been studied in vitro and in vivo. CB, CF, and BE sputa are transported slowly by the mammalian ciliated epithelium of the mucus depleted bovine trachea (MDBT) (Wills et al, J. Clin. Invest. 97(1): 9-13, 1995). Slow transportability of diseased sputum on the MDBT may be linked to its low electrolyte/osmolyte content (Wills et al, J. Resp. Crit. Care Med. 151(4): 1255-1258, 1997). Indeed, diseased sputum is known to have low electrolyte content relative to plasma (Matthews et al, Am. Rev. Resp. Dis. 88: 199-204, 1963; Potter et al, Am. Rev. Resp. Dis. 67(1): 83-87, 1967; Tomkiewicz et al, Am. Rev. Resp. Dis. 148(4, Pt. 1): 1002-1007, 1993).

Further studies on the MDBT have shown that transportability of diseased mucus is markedly improved following treatment with sodium chloride (Wills et al 1995). Furthermore, clinical studies have shown that inhalation of hypertonic saline, or of the epithelial sodium channel (ENaC) blocker amiloride can significantly increase MCC in diseased patients (Robinson et al, Thorax 52(10): 900-903, 1997; App et al, Am. Rev. Resp. Dis. 141, 605-612, 1990). Recently, the relationship between mucus clearance and its ionic composition in vivo in the guinea-pig model of tracheal mucus velocity (TMV) has been elucidated. In vivo studies showed that a 5 minute aerosol of hypertonic saline transiently increased TMV. An increase in TMV was observed 1 min after hypertonic saline (14.4%) aerosol. TMV was 5.1±1.0 mm.min⁻¹ (n=9) in 0.9% saline-exposed animals compared to 11.3±1.3 mm.min⁻¹ in hypertonic saline exposed animals (n=9; p≦0.001)(Newton & Hall, 1997). Inhaled amiloride also caused an increase in TMV. A significant increase in TMV was observed 15 minutes after a 20 minute aerosol of amiloride (10 mM). TMV was 3.2±2.5 mm.min⁻¹ (n=9) in water-exposed animals compared to 8.1±0.3 mm.min⁻¹ in amiloride-exposed animals (n=8; p≦0.05) Newton et al, Ped. Pulm. S17, Abs. 364, 1998). These agents would appear to act by increasing the ionic content of airway surface liquid (ASL).

Clinical genetic evidence from subjects with systemic pseudohypo-aldosteronism (SPHA) also supports the view that down-regulation of the activity of airway epithelial sodium channels will increase mucociliary clearance in the lung. SPHA patients with loss of function mutations in the genes for the epithelial sodium channel subunits had no sodium absorption from airways surfaces, they had increased sodium ion concentration in nasal surface liquid compared to normal subjects, and they had 4-fold increased lung mucociliary clearance rate compared to normal subjects (Kerem et al, New England J. Med. 341, 156-162, 1999).

Recently, a serine protease termed channel activating protease-1 (CAP-1) has been found in the apical membrane of amphibian Xenopus kidney epithelial cells (A6 cells) (Vallet et al, Nature 389(6651): 607-610, 1997). CAP-1 appears to modulate Na⁺ channel activity in these cells. Exposure of the apical membrane to the prototypical bovine Kunitz inhibitor, aprotinin, reduced transepithelial Na⁺ transport (Vallet et al 1997: Chraibi et al, J. Gen. Physio. 111(1): 127-138, 1998). The effect of Bukinin (1-170), a two Kunitz domain human homologue of bovine aprotinin (Delaria et al, J. Biol. Chem. 272(18): 12209-12214, 1997; Marlor et al, J. Biol. Chem. 272(18): 12202-12208, 1997), was evaluated using normal cultured human bronchial epithelial cell (HBE) short circuit current (Isc) in vitro (McAulay et al, Ped. Pulm. S17, Abs. 141, 1998). Bikunin (1.5 ug.ml⁻¹: 70 nM) significantly inhibited 54% Na⁺ Isc in normal HBE cells (n=5-8; p≦0.05). Overall, Bikunin (70 nM) inhibited 58% of the baseline Isc in 90 minutes. In a further study, Bikunin (5 ug.ml⁻¹) significantly inhibited 84% Na⁺ Isc in normal HBE cells (n=6; p≦0.01) whilst the serpin-family serine protease inhibitor alpha(1)-protease inhibitor (α₁-PI) (50 ug.ml⁻¹) was without a significant effect. In cultured human cystic fibrosis airway epithelial cells in vitro, Isc was inhibited by bikunin (1-170) (1 ug/mL), and was inhibited by aprotinin (20 ug/mL).

Two recent studies by a single research group have demonstrated a protease inhibitor induced effect on TMV α₁-PI (10 mg) given either 30 min before antigen challenge, or 1 h after challenge, attenuated antigen-induced reduction in TMV in allergic sheep, 6 h after challenge (O'Riordan et al, Am. J. Resp. Crit. Care Med. 97(5): 1522-1528, 1997). In FIG. 1 in the O'Riordan et al 1997 paper, the authors showed that α₁-PI administered on its own (no antigen challenge) to the airways of allergic sheep, had no effect on baseline TMV over a 6 h period. In the second study, α₁-PI was given 6 h after antigen challenge and caused only a significant reversal of the antigen-induced fall in TMV at 24 h after challenge (O'Riordan et al, J. App. Physio. 85(3): 1086-1091, 1998). The authors argue that the mechanism for the effect of α₁-PI is associated with its anti-neutrophil elastase property, where neutrophil elastase is believed to be the enzyme responsible for the reduced rate of mucociliary clearance in their model. They reasoned that a1-PI could be used to treat mucociliary dysfunction brought about by allergy-induced neutrophil elastase release in asthma (O'Riordan et al 1998); they did not speculate on a potential role in other respiratory diseases.

### BRIEF SUMMARY OF THE INVENTION

The instant invention is directed to the use of Kunitz-family serine protease inhibitors that stimulate the rate of mucociliary clearance (MCC) of mucus and sputum in the airways of the lung. Kunitz-serine protease inhibitors can be used to treat lung diseases such as Cystic Fibrosis (CF), Chronic Bronchitis (CB) and Bronchiectasis (BE) where the retention and accumulation of mucus is a major clinical problem. Until now, prior art has not associated protease inhibitors with the ability to increase the rate of MCC above baseline rate. Kunitz-type serine protease inhibitors can also be used to treat chronic sinusitis and glue ear where the retention and accumulation of mucus is a clinical problem.

The instant invention contemplates the use of serine protease inhibitor proteins which include Kunitz domains or Kunitz-like domains for use in a method for stimulating MCC. In one embodiment of the invention, bovine serine protease inhibitor proteins such as aprotinin and variants and fragments thereof such as the ones described in EP 821007, published Jan. 28, 1998 may be used in practicing the invention.

In another embodiment of the invention, human serine protease inhibitors are contemplated for use in the method for stimulating the rate of MCC. Representative examples of human serine protease inhibitors include Bikunin and variants and fragments thereof such as the ones described in WO 97/33996, published Sep. 18, 1997 (Bayer Corp.), and U.S. Pat. No. 5,407,915, issued Apr. 18, 1995 (Bayer AG) which are incorporated herein in their entirety.

### DESCRIPTION OF THE DRAWINGS

The invention will be better understood from a consideration of the following detailed description and claims, taken in conjunction with the drawings, in which:

FIG. 1 depicts the nucleotide sequence of EST R35464 (SEQ ID NO.: 12) and the translation of this DNA sequence (SEQ ID NO.: 13) which yielded an open reading frame with some sequence similarity to aprotinin. The translation product contains 5 of the 6 cysteines in the correct spacing that is characteristic for Kunitz-like inhibitor domains (indicated in bold). The position normally occupied by the remaining cysteine (at codon 38) contained instead a phenylalanine (indicated by an asterisk).

FIG. 2 depicts the nucleotide sequence of EST R74593 (SEQ ID NO.: 14), and the translation of this DNA sequence (SEQ ID NO.: 15) which yielded an open reading frame with homology to the Kunitz class of serine protease inhibitor domains. The translation product contained 6 cysteines in the correct spacing that is characteristic for Kunitz-like inhibitor domains (indicated in bold). However, this reading frame sequence includes stop codons at codon 3 and 23.

FIG. 3 depicts a deduced nucleic acid sequence of human placental bikunin (SEQ ID NO.: 9) labeled “consensus” and matched with the translated protein amino acid sequence labeled “translated” (SEQ ID NO.: 10). Also as comparison are shown the nucleic acid sequence for ESTs H94519 (SEQ ID NO.: 16), N39798 (SEQ ID NO.: 17), R74593 (SEQ ID NO.: 14) and R35464 (SEQ ID NO.: 12). The underlined nucleotides in the consensus sequence correspond to the site of PCR primers described in the Examples. Underlined amino acids in the translated consensus sequence are residues whose identity have been confirmed by amino acid sequencing of purified native human placental bikunin. Nucleotide and amino acid code are standard single letter code, “N” in the nucleic acid code indicates an unassigned nucleic acid, and “*” indicates a stop codon in the amino acid sequence.

FIG. 4A depicts the original overlay of a series of ESTs with some nucleic acid sequence homology to ESTs encoding human placental bikunin, or portions thereof. Shown for reference are the relative positions of bikunin (7-64) and bikunin (102-159), labeled KID1 and KID2 respectively.

FIG. 4B depicts a subsequent more comprehensive EST overlay incorporating additional ESTs. Numbers on the upper X-axis refer to length in base pairs, starting at the first base from the most 5′ EST sequence. The length of each bar is in proportion to the length in base pairs of the individual ESTs including gaps. The EST accession numbers are indicated to the right of their respective EST bars.

FIG. 4C depicts the corresponding alignment of the oligonucleotide sequences of each of the overlapping ESTs shown schematically in FIG. 4B. The upper sequence (SEQ ID NO.: 51) labeled bikunin represents the consensus oligonucleotide sequence derived from the overlapping nucleotides at each position. The numbers refer to base-pair position within the EST map. The oligonucleotides in EST R74593 that are bold underlined (at map positions 994 and 1005) are base insertions observed in R74593 that were consistently absent in each of the other overlapping ESTs.

FIG. 4D depicts the amino acid translation of the consensus oligonucleotide sequence for bikunin depicted in FIG. 4C (SEQ ID NO.: 45).

FIG. 4E depicts the nucleotide sequence (SEQ ID NO.: 46) and corresponding amino acid translation (SEQ ID NO.: 47) of a placental bikunin encoding sequence that was derived from a human placental cDNA library by PCR-based amplification.

FIG. 4F depicts the nucleotide sequence (SEQ ID NO.: 48) and corresponding amino acid translation (SEQ ID NO.: 49) of a native human placental bikunin encoding clone that was isolated from a human placental lambda cDNA library by colony hybridization.

FIG. 4G compares the alignment of the amino acid translated oligonucleotide sequences for placental bikunin obtained by EST overlay (SEQ ID NO.: 45), PCR based cloning (SEQ ID NO.: 47), and conventional lambda colony hybridization (SEQ ID NO.: 49).

FIG. 5 shows a graph of purification of human placental bikunin from placental tissue after Superdex 75 Gel-Filtration. The plot is an overlay of the protein elution profile as measured by OD 280 nm (solid line), activity of eluted protein in a trypsin inhibition assay (% inhibition shown by circles), and activity of eluted protein in a kallikrein inhibition assay (% inhibition shown by squares).

FIG. 6 shows a graph which plots the purification of human placental bikunin from placental tissue using C18 Reverse-Phase Chromatography. The plot is an overlay of the protein elution profile as measured by OD 215 nm (solid line), activity of eluted protein in a trypsin inhibition assay (% inhibition shown by circles), and activity of eluted protein in a kallikrein inhibition assay (% inhibition shown by squares).

FIG. 7 depicts a silver stained SDS-PAGE gel of highly purified placental bikunin (lane 2), and a series of molecular size marker proteins (lane 1) of the indicated sizes in kilodaltons. Migration was from top to bottom.

FIG. 8 shows the amount of trypsin inhibitory activity present in the cell-free fermentation broth from the growth of yeast strains SC101 (panel 8A) or WHL341 (panel 8B) that were stably transformed with a plasmid (pS604) that directs the expression of placental bikunin (102-159).

FIG. 9 shows both a silver stained SDS-PAGE (FIG. 9A) and a Western blot with anti-placental bikunin (102-159) pAb (FIG. 9B) of cell-free fermentation broth from the growth of yeast strain SC101 (recombinants 2.4 and 2.5) that was stably transformed with a plasmid directing the expression of either bovine aprotinin, or placental bikunin (102-159). Migration was from top to bottom.

FIG. 10 is a photograph which shows a silver stained SDS-PAGE of highly purified placental bikunin (102-159) (lane 2) and a series of molecular size marker proteins (lane 1) of the indicated sizes in Kilodaltons. Migration was from top to bottom.

FIG. 11 is a photograph which shows the results of Northern blots of mRNA from various human tissues that was hybridized to a ³²P labeled cDNA probe encoding either placental bikunin (102-159) (FIG. 11A) or encoding placental bikunin (1-213) (FIG. 11B). Migration was from top to bottom. The numbers to the right of each blot refer to the size in kilobases of the adjacent RNA markers. The organs from which mRNA was derived is described under each lane of the blot.

FIG. 12 depicts an immunoblot of placental derived placental bikunin with rabbit antiserum raised against either synthetic reduced placental bikunin (7-64) (FIG. 12A) or 102-159 (FIG. 12b). For each panel, contents were: molecular size markers (lanes 1); native placental bikunin isolated from human placenta (lanes 2); synthetic placental bikunin (7-64) (lanes 3) and synthetic placental bikunin (102-159) (lanes 4). Tricine 10-20% SDS-PAGE gels were blotted and developed with protein A-purified primary polyclonal antibody (8 ug IgG in 20 ml 0.1% BSA/Tris-buffered saline (pH 7.5), followed by alkaline phosphatase-conjugated goat anti-rabbit secondary antibody. Migration was from top to bottom.

FIG. 13 depicts a Coomassie Blue stained 10-20% Tricine SDS-PAGE gel of 3 micrograms of highly purified placental bikunin (1-170) derived from a baculovirus/Sf9 expression system (lane 2). Lane 1 contains molecular size markers. Migration was from top to bottom.

FIG. 14 depicts a comparison of the effect of increasing concentrations of either Sf9-derived human placental bikunin (1-170) (filled circles), synthetic placental bikunin (102-159) (open circles), or aprotinin (open squares) on the activated partial thromboplastin time of human plasma. Clotting was initiated with CaCl₂. The concentration of proteins are plotted versus the fold prolongation in clotting time. The uninhibited clotting time was 30.8 seconds.

FIG. 15 illustrates the effect of Bikunin at dosage levels of 2 uM and 0.2 uM relative to amiloride (100 uM) and Hank's Balanced salt solution (HBSS) vehicle (control) on potential differences in guinea pig trachea 3 hours post-treatment.

FIG. 16 illustrates (a) the positioning of the instillment syringe and beta probe relative to the guinea pig trachea; (b) a representative graph for measurement of trachea mean velocity (TMV) using ³²P-labelled S. cerevisae; and (c) the sustained increase in TMV in vivo in guinea pig in response to Bikunin (5 ug) relative to HBSS vehicle control at 1.5, 1.75, 2.0, 2.25 and 2.5 hours following tracheal instillment.

FIG. 17 illustrates that Bikunin (70 nM) decreases sodium current in cultured human bronchial epithelial cells in vitro relative to amiloride (10 uM).

FIG. 18 illustrates the effect of a 5 min aerosol of hypertonic saline (14.4%) on increasing TMV, following aerosol treatment in guinea pig trachea.

FIG. 19 illustrates the effect of a 20 minute aerosol of amiloride (10 mM) on TMV, following aerosol treatment in guinea pig trachea.

FIG. 20 illustrates that Bikunin (5 ug/mL), aprotinin (5 ug/mL), and aprotinin double mutein (0.5 ug/mL, 1.5 ug/mL and 5 ug/mL) decrease sodium short circuit current in cultured human bronchial cells in vitro.

FIG. 21 illustrates that Aprotinin (1 mg/mL) inhibited Isc in vitro in human CF bronchial epithelial cells.

FIG. 22 illustrates that Bikunin aerosol (3 mL of 3 mg/mL) significantly increased TMV in sheep relative to PBS control.

FIG. 23 illustrates that Bikunin (50 ug/mL) inhibited sodium current in vitro in guinea pig tracheal epithelial cells over a 30 minute period.

FIG. 24 illustrates that Bikunin (100 ug/mL) significantly inhibited sodium current in vitro in ovine trachael epithelial cells over a 90 minute period.

FIG. 25 illustrates that (a) exposure to LPS caused a significant PMN influx and that (b) Bikunin significantly inhibited potential difference in guinea pigs pre-exposed to LPS.

FIG. 26 illustrates that Aprotinin double mutein (10 ug) increased TMV in vivo in the guinea pig relative to HBSS over a sustained period of 1.5 to 2.5 hours following administration.

FIG. 27 represents a plasmid map of pBC-BK (CMV-IE=cytomegalovirus immediate early; DHFR=dihydrofolate reductase; AMP-r=ampicillin resistance)

FIG. 28 illustrates that (a) CHO expressed Bikunin (1-170) (10 ug/mL) decreases sodium current in vitro in human CF bronchial epithelial cells over a 90 minute period and (b) CHO Bikunin (1-170) at 1, 5, and 10 ug/mL and Aprotinin at 20 ug/mL decreases sodium current at 90 minutes after apical application to human CF bronchial epithelial cells in vitro.

FIG. 29 illustrates the process steps for purifying Bikunin (1-170) from a CHO cell expression system.

FIG. 30A shows a graph of the migration of the isoforms of purified CHO bikunin (1-170) using C18 Reverse-Phase Chromatography. The plot is an overlay of the protein elution profile as measured by Absorbance at 280 nm (solid line) and the percentage of acetonitrile in 0.1% Trifluoroacetic acid used to elute the protein (diamonds).

FIG. 30B is a photograph which shows a silver stained SDS-PAGE of purified bikunin (1-170) glycosylated isoforms (lanes 45-55) expressed from a CHO cell expression system and a series of molecular size marker proteins (between lanes 54 and 55) of the indicated sizes in Kilodaltons. Migration was from top to bottom.

FIG. 31 is a photograph which shows a silver stained SDS-PAGE of N-Glycosidase F treated (lanes 2 and 4) and untreated (lanes 1 and 3) of CHO purified bikunin (1-170) isoforms and a series of molecular size marker proteins of the indicated sizes in Kilodaltons. Migration was from top to bottom.

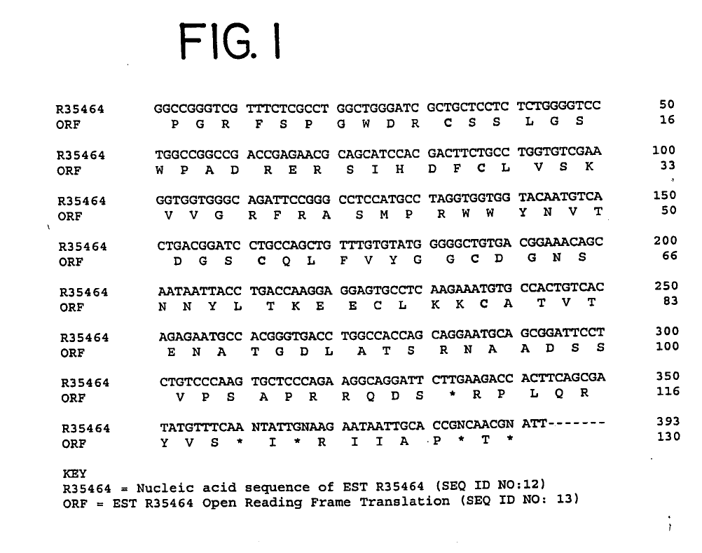

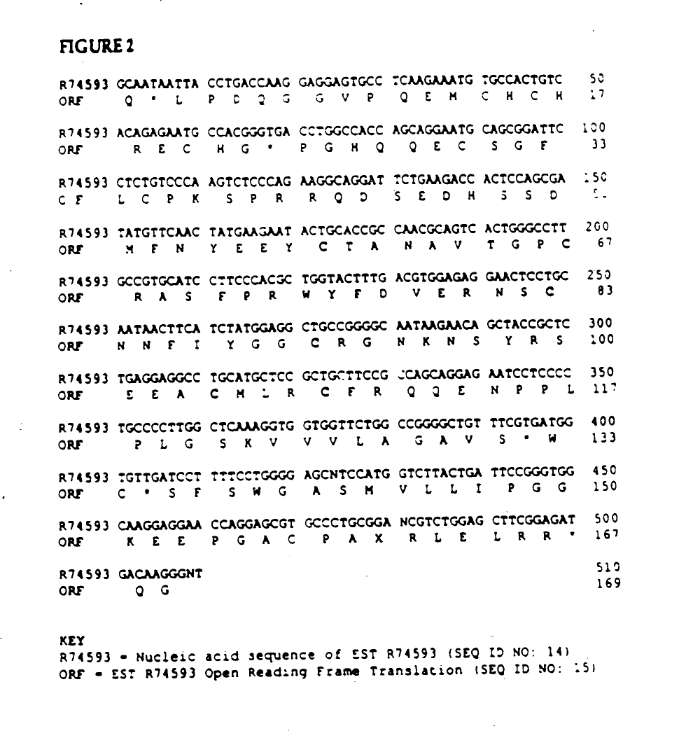

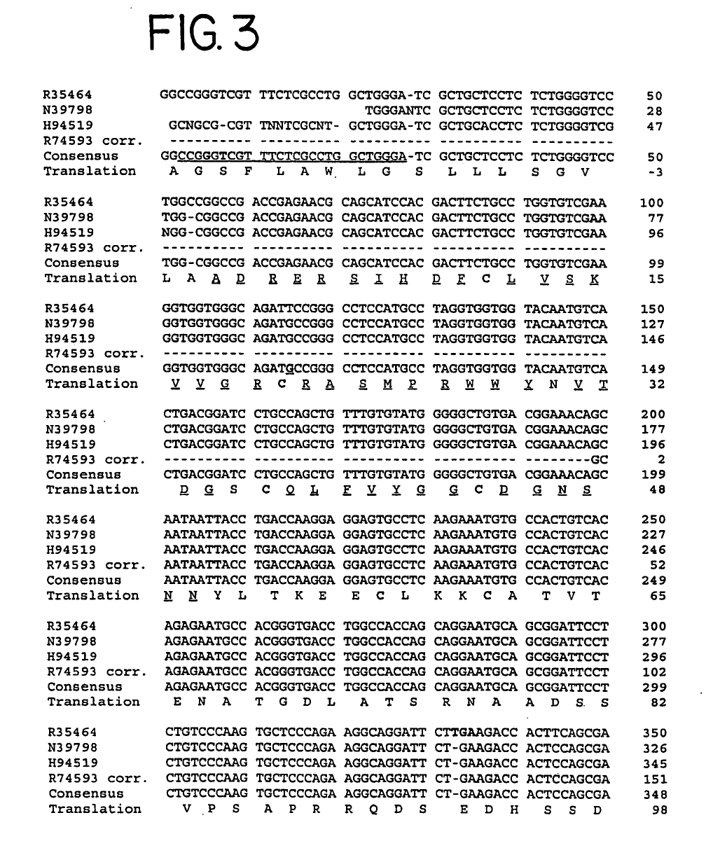

### DETAILED DESCRIPTION OF THE INVENTION

The present invention relates to compositions comprising Kunitz-type serine protease inhibitor proteins and fragments thereof which stimulate the rate of mucociliary clearance of mucus and sputum in lung airways. The compositions also encompass a newly identified human protein herein called human placental bikunin that contains two serine protease inhibitor domains of the Kunitz class.

The present invention also provides methods for stimulating the rate of mucociliary clearance in patients with mucociliary dysfunction, wherein an effective amount of the disclosed serine protease inhibitors of the present invention, in a biologically compatible vehicle, is administered to the patient.

A preferred application for placental bikunin, isolated domains, and other variants is for stimulating mucociliary clearance in CF patients as part of disease therapy and management. These methods and compositions reduce or eliminate mucus and sputum buildup in lung airways in patients with chronic obstructive lung disease, thereby reducing the risk of secondary lung infections and other adverse side effects, as well as avoiding or delaying the need for lung transplant surgery in CF patients.

The method of the present invention contemplates the use of aprotinin to stimulating MCC. Aprotinin has been shown to reduce transepithelial Na⁺ transport in the apical membrane of amphibian Xenopus kidney epithelial cells (A6 cells) (Vallet et al 1997: Chraibi et al 1998). The mechanism of aprotinin action has been proposed to involve inhibition of CAP-1, a protease involved in modulating Na⁺ channel activity in A6 cells. Bikunin (1-170), a two Kunitz domain human homologue of bovine aprotinin (Delaria et al 1997: Marlor et al 1997), was also shown to significantly inhibit normal cultured human bronchial epithelial cell (HBE) short circuit current (Isc) in vitro (McAulay et al 1998). Bikunin (1.5 ug.ml⁻¹: 70 nM) significantly inhibited 54% Na⁺ Isc in normal HBE cells (n=5-8; p≦50.05). Overall, Bikunin (70 nM) inhibited 58% of the baseline Isc in 90 minutes. In a further study, Bikunin (5 ug.^{ml-}1) significantly inhibited 84% Na⁺ Isc in normal HBE cells (n=6; p≦50.01) whilst the serpin-family serine protease inhibitor alpha(1)-protease inhibitor (α₁-PI)(50 ug.mL⁻¹) was without a significant effect. In cultured human cystic fibrosis airway epithelial cells in vitro, Isc was inhibited by bikunin (1-170) (1 ug/mL), and was inhibited by aprotinin (20 ug/mL).

In light of these observations, Kunitz-type serine inhibitors such as aprotinin, placental bikunin and fragments thereof are contemplated as therapeutics for treating mucocililiary dysfunction, including cystic fibrosis.

By “Kunitz inhibitor” is meant an inhibitor of proteases; structurally, a “Kunitz inhibitor” or “Kunitz domain” is a protein, or protein domain, typically of about 60 amino acids in length and containing three disulfide bonds. (See Laskowske & Kato, Ann. Rev. Biochem. 49, 593-626, 1980).

A significant advantage of the Kunitz domains of the serine protease inhibitor Bikunin and fragments and analogs thereof of the present invention is that they are human proteins, and also less positively charged than Trasylol® (Example 1), thereby reducing the risk of kidney damage on administration of large doses of the proteins. Being of human origin, the protein of the instant invention can thus be administered to human patients with significantly reduced risk of undesired immunological reactions as compared to administration of similar doses of Trasylol®. Furthermore, it was found that bikunin (102-159), bikunin (7-64), and bikunin (1-170) are significantly more potent inhibitors of plasma kallikrein than Trasylol® in vitro (Example 3, 4 and 10). Thus bikunin and fragments thereof are expected to be more effective in vivo relative to aprotinin.

The amount of the pharmaceutical composition to be employed will depend on the recipient and the condition being treated. The requisite amount may be determined without undue experimentation by protocols known to those skilled in the art. Alternatively, the requisite amount may be calculated, based on a determination of the amount of target protease such as plasmin, kallikrein or prostasin which must be inhibited in order to treat the condition. As the active materials contemplated in this invention are deemed to be nontoxic, treatment preferably involves administration of an excess of the optimally required amount of active agent.

For stimulating the rates of mucociliary clearance in patients with chronic obstructive lung disease, the proteins of the instant invention may be used like aprotinin Trasylol® while taking into account the differences in potency. The use of Trasylol®t is outlined in the Physicians Desk Reference, 1995, listing for Trasylol® supplement A. Briefly, with the patient in a supine position, the loading dose of placental bikunin, isolated domain or other variant is given by infusion slowly over about 20 to 30 minutes. In general, a total dose of between about 2×10⁶ KIU (kallikrein inhibitory units) and 8 X10⁶ KIU will be used, depending on such factors as patient weight and condition. Preferred loading doses are those that contain a total of 1 to 2 million kallikrein inhibitory units (KIU).

The proteins of the instant invention are employed in pharmaceutical compositions formulated in the manner known to the art. Such compositions contain active ingredient(s) plus one or more pharmaceutically acceptable carriers, diluents, fillers, binders, and other excipients, depending on the administration mode and dosage form contemplated. Examples of therapeutically inert inorganic or organic carriers known to those skilled in the art include, but are not limited to, lactose, corn starch or derivatives thereof, talc, vegetable oils, waxes, fats, polyols such as polyethylene glycol, water, saccharose, alcohols, glycerin and the like. Various preservatives, emulsifiers, dispersants, flavorants, wetting agents, antioxidants, sweeteners, colorants, stabilizers, salts, buffers and the like can also be added, as required to assist in the stabilization of the formulation or to assist in increasing bioavailability of the active ingredient(s) or to yield a formulation of acceptable flavor or odor in the case of oral, nasal or pulmonary dosing. The inhibitor employed in such compositions may be in the form of the original compound itself, or optionally, in the form of a pharmaceutically acceptable salt. The compositions so formulated are selected as needed for administration of the inhibitor by any suitable mode known to those skilled in the art.

Parenteral administration modes include intravenous (i.v.), subcutaneous (s.c.), intraperitoneal (i.p.), and intramuscular (i.m.) routes. Intravenous administration can be used to obtain acute regulation of peak plasma concentrations of the drug as might be needed. Alternatively, the drug can be administered at a desired rate continuously by i.v. catheter. Suitable vehicles include sterile, non-pyrogenic aqueous diluents, such as sterile water for injection, sterile-buffered solutions or sterile saline. The resulting composition is administered to the patient prior to and/or during surgery by intravenous injection or infusion.

Improved half life and targeting of the drug to phagosomes such as neutrophils and macrophage involved in inflammation may be aided by entrapment of the drug in liposomes. It should be possible to improve the selectivity of liposomal targeting by incorporating into the outside of the liposomes ligands that bind to macromolecules specific to target organs/tissues such as the GI tract and lungs. Alternatively, i.m. or s.c. deposit injection with or without encapsulation of the drug into degradable microspheres (e.g., comprising poly-DL-lactide-co-glycolide) or protective formulations containing collagen can be used to obtain prolonged sustained drug release. For improved convenience of the dosage form it is possible to use an i.p. implanted reservoir and septum such as the percuseal system. Improved convenience and patient compliance may also be achieved by use of either injector pens (e.g., the Novo Pin or Q-pen) or needle-free jet injectors (e.g., from Bioject, Mediject or Becton Dickinson). Precisely controlled release can also be achieved using implantable pumps with delivery to the desired site via a cannula. Examples include the subcutaneously implanted osmotic pumps available from ALZA such as the ALZET osmotic pump.

Oral delivery may be achieved by incorporating the drug into tablets, coated tablets, dragees, hard and soft gelatin capsules, solutions, emulsions, suspensions or enteric coated capsules designed to release the drug into the colon where digestive protease activity is low. Examples of the latter include the OROS-CT/Osmet™ system of ALZA, and the PULSINCAP™ system of Scherer Drug Delivery Systems. Other systems use azo-crosslinked polymers that are degraded by colon-specific bacterial azoreductases, or pH sensitive polyacrylate polymers that are activated by the rise in pH in the colon. The above systems may be used in conjunction with a wide range of available absorption enhancers. Rectal delivery may be achieved by incorporating the drug into suppositories. Nasal delivery may be achieved by incorporating the drug into bioadhesive particulate carriers (<200 mm) such as those comprising cellulose, polyacrylate or polycarbophil, in conjunction with suitable absorption enhancers such as phospholipids or acylcarnitines. Commercially available systems include those developed by Dan Biosys and Scios Nova.

For stimulating the rate of mucociliary clearance, the preferred mode of administration of the placental bikunin variants of the present invention is pulmonary delivery. The Kunitz-type serine protease inhibitors disclosed herein may be administered to the lungs of a subject by any suitable means, but are preferably administered by administering an aerosol suspension of respirable particles comprised of the active compound, which the subject inhales. The respirable particles may be liquid or solid. Micron-sized dry powders containing the medicament in a suitable carrier such as mannitol, sucrose or lactose may be delivered to the lung airway surface using dry powder inhalers such as those of Inhale™, Dura™, Fisons (Spinhaler™), and Glaxo (Rotahaler™), or Astra (Turbohaler™) propellant based metered dose inhalers. Solution formulations with or without liposomes may be delivered using nebulizers.

Aerosols of liquid particles comprising the proteins may be produced by any suitable means, such as with a pressure-driven aerosol nebulizer or an ultrasonic nebulizer. See, e.g., U.S. Pat. No. 4,501,729. Nebulizers are commercially available devices which transform solutions or suspensions of the active ingredient into a therapeutic aerosol mist either by means of acceleration of compressed gas, typically air or oxygen, through a narrow venturi orifice or by means of ultrasonic agitation. Suitable formulations for use in nebulizers consist of the active ingredient in a liquid carrier. The carrier is typically water (and most preferably sterile, pyrogen-free water) or a dilute aqueous alcoholic solution, preferably made isotonic with body fluids by the addition of, for example, sodium chloride. Optional additives include preservatives if the formulation is not made sterile, for example, methyl hydroxybenzoate, antioxidants, flavoring agents, volatile oils, buffering agents and surfactants.

Aerosols of solid particles comprising the protein may likewise be produced with any solid particulate medicament aerosol generator. Aerosol generators for administering solid particulate medicaments to a subject produce particles which are respirable, as explained above, and generate a volume of aerosol containing a predetermined metered dose of a medicament at a rate suitable for human administration. One illustrative type of solid particulate aerosol generator is an insufflator. Suitable formulations for administration by insufflation include finely comminuted powders which may be delivered by means of an insufflator or taken into the nasal cavity in the manner of a snuff. In the insufflator, the powder (e.g., a metered dose thereof effective to carry out the treatments described herein) is contained in capsules or cartridges, typically made of gelatin or plastic, which are either pierced or opened in situ and the powder delivered by air drawn through the device upon inhalation or by means of a manually-operated pump. The powder employed in the insufflator consists either solely of the protein or of a powder blend comprising the protein, a suitable powder diluent, such as lactose, and an optional surfactant. A second type of illustrative aerosol generator comprises a metered dose inhaler. Metered dose inhalers are pressurized aerosol dispensers, typically containing a suspension or solution formulation of the active ingredient in a liquified propellant. During use these devices discharge the formulation through a valve adapted to deliver a metered volume, typically from 10 to 200 uL, to produce a fine particle spray containing the protein. Suitable propellants include certain chlorofluorocarbon compounds, for example, dichlorodifluoromethane, trichlorofluoromethane, dichlorotetrafluoroethane and mixtures thereof. The formulation may additionally contain one or more co-solvents, for example, ethanol, surfactants, such as oleic acid or sorbitan trioleate, antioxidants and suitable flavoring agents.

For metered dose inhaler or dry powder inhaler devices, the aerosol, whether formed from solid or liquid particles, may be produced by the aerosol generator at a rate of from about 5 to 150 liters per minute, more preferably from about 10 to 100 liters per minute, and most preferably for metered dose inhalers from about 10 to 50 liters per minute, and most preferably for dry powder inhalers about 60 liters per, minute. Aerosols generated by nebulizer, jet or ultrasonic, may be produced by the aerosol generator at a rate of from about 1 to 100 liters per minute, more preferably from about 4 to 10 liters per minute. Aerosols containing greater amounts of protein may be administered more rapidly.

The dosage of the protease inhibitor will vary depending on the condition being treated and the state of the subject. The daily dose may be divided among one or several unit dose administrations. The daily dose by weight may range from about 0.01 to 20 milligrams of respirable particles for a human subject, depending upon the age and condition of the subject.

Solid or liquid particulate pharmaceutical formulations containing protease inhibitors of the present invention should include particles of respirable size: that is, particles of a size sufficiently small to pass through the mouth and larynx upon inhalation and into the bronchi and alveoli of the lungs. In general, particles ranging from about 1 to 8 microns in size (more particularly, less than about 6 microns in size) are respirable. Particles of non-respirable size which are included in the aerosol tend to be deposited in the throat and swallowed, and the quantity of non-respirable particles in the aerosol is preferably minimized. For nasal administration, a particle size in the range of 10-500 microns is preferred to ensure retention in the nasal cavity.

In the manufacture of a formulation according to the invention, the protease inhibitor is typically admixed with, inter alia, an acceptable carrier. The carrier must, of course, be acceptable in the sense of being compatible with any other ingredients in the formulation and must not be deleterious to the patient. The carrier may be a solid or a liquid, or both, and is preferably formulated with the compound as a unit-dose formulation, for example, a capsule, which may contain from 0.5% to 99% by weight of the active compound. One or more active compounds may be incorporated in the formulations of the invention, which formulations may be prepared by any of the well-known techniques of pharmacy consisting essentially of admixing the components.

Compositions containing respirable dry particles of protease inhibitor may be prepared by grinding the inhibitor with a mortar and pestle, and then passing the micronized composition through a 400 mesh screen to break up or separate out large agglomerates.

The pharmaceutical composition may optionally contain a dispersant which serves to facilitate the formation of an aerosol. A suitable dispersant is lactose, which may be blended with the active agent in any suitable ratio (e.g., a 1 to 1 ratio by weight).

If desired, general ex vivo and in vivo gene therapy strategies may employed to deliver nucleic acid constructs encoding Kunitz-type serine protease inhibitor proteins such as Bikunin, Aprotinin or fragments and variants thereof such as the ones described in WO 97/33996 (Bayer Corp.) and U.S. Pat. No. 5,407,915. (Bayer AG). Gene therapy techniques that are primarily virus-based have been used to transform pulmonary cells as a means for treating the manifestations of CF in the lung and associated extrapulmonary tissues. See WO 93/03709, published Mar. 3, 1993 which describes the use of retroviral and non-retroviral vectors (e.g., adenoviruses and adeno-associated viruses) for the stable expression of the CFTR gene in CF patients. Alternatively, non-viral methods for delivery of exogenous nucleic acids are also known and are contemplated for use in the instant invention. See WO 93/12240, published Jun. 24, 1993 and references cited therein, describing a transcription or expression cassettes including the coding sequence for a CFTR molecule operably joined to regulatory sequences functional in a mammal. The nucleic acid constructs are then supplied to the airways and alveoli of the lung in a number of ways including aerosolized delivery alone or in combination with lipid-based complexes, e.g., Lipofectin™ WO 95/26356, published Oct. 5, 1995 describes representative examples of lipids useful for transfection. It is therefore contemplated in the instant invention that nucleic acid molecules encoding Kunitz-type serine protease inhibitors such as Bikunin, Aprotinin or variants and fragments thereof may be similarly administered to lung airways by any suitable gene therapy method as a means for stimulating the rate of mucociliary clearance of mucus and sputum in a subject in need of such treatment.

### Searching Human Sequence Data

The existence of a distinct human protein homologous in function to aprotinin, was deduced following a unique analysis of sequence entries to the expressed-sequence-tag data-base (hereafter termed dbEST) at the NCBI (National Center for Biological Information, Maryland). Using the TBlastN algorithm (BLAST, or Basic Local Alignment Search Tool uses the method of Altschul et al., (1990) J. Mol. Biol 215, 00 403-410, to search for similarities between a query sequence and all the sequences in a data-base, protein or nucleic acid in any combination), the data-base was examined for nucleotide sequences bearing homology to the sequence of bovine pre-pro-aprotinin, Trasylol®. This search of numerous clones was selectively narrowed to two particular clones which could possibly encode for a deduced amino acid sequence that would correspond to a human protein homologous in function to aprotinin. The selected nucleic acid sequences were R35464 (SEQ ID NO: 12) and R74593 (SEQ ID NO: 14) that were generated from a human placental nucleic acid library. The translated protein sequence in the longest open reading frame for R35464 (SEQ ID NO: 13) was missing one of the 6 cysteines that are critical for formation of the Kunitz-domain covalent structure, meaning that the nucleic acid sequence of R35464 could not yield a functional inhibitor. Similarly, the longest translated open reading frame from clone R74593 (SEQ ID NO: 15) contained a stop codon 5′ to the region encoding the Kunitz like sequence, meaning that this sequence, could not be translated to yield a functional secreted Kunitz domain. The significance of these sequences alone was unclear. It was possible that they represented a) the products of pseudogenes, b) regions of untranslated mRNA, or c) the products of viable mRNA which had been sequenced incorrectly.

### Discovery of Human Bikunin

To specifically isolate and determine the actual human sequence, cDNA primers were designed to be capable of hybridizing to sequences located 5′ and 3′ to the segment of cDNA encoding our proposed Kunitz like sequences found within R35464 and R74593. The primers used to amplify a fragment encoding the Kunitz like sequence of R74593 were CGAAGCTTCATCTCCGAAGCTCCAGACG (the 3′primer with a HindIII site; SEQ ID NO.: 33) and AGGATCTAGACAATAATTACCTGACCAAGGA (the 5′primer with an XbaI site; SEQ ID NO.: 34).

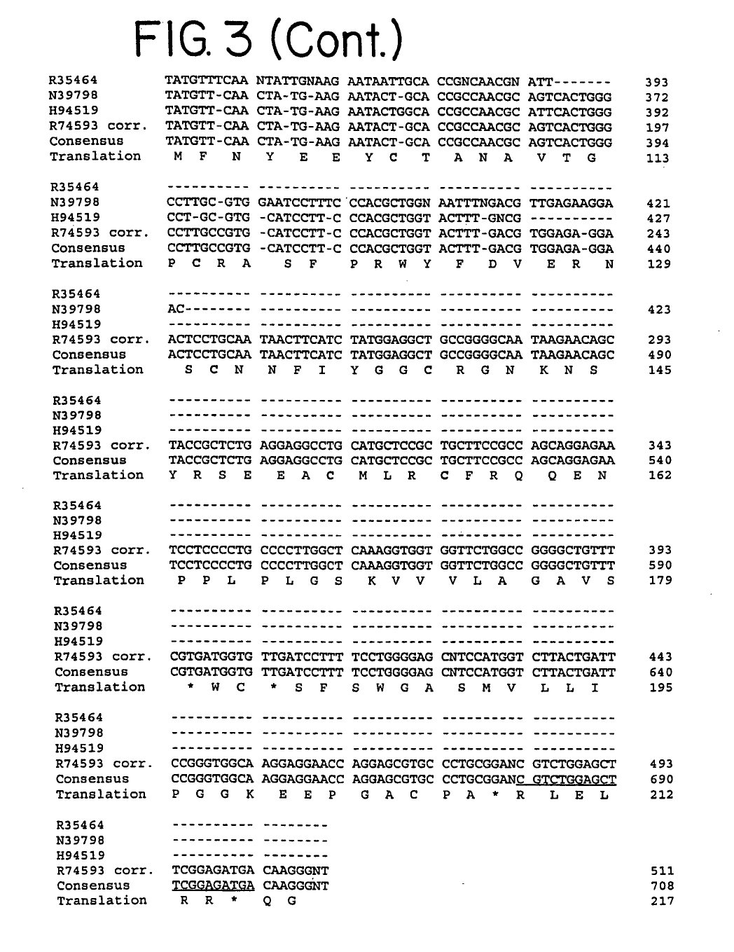

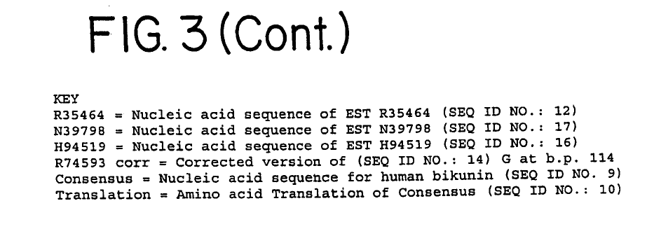

These primers were used to amplify by PCR (30 cycles) a 500 base pair product from a human placental cDNA library from Clontech (MATCHMAKER, Cat #HL4003AB, Clontech Laboratories, Palo Alto, Calif.), which was subcloned into Bluescript-SK+ and sequenced with the T3 primer with a Sequenase™ kit version 2.0. Surprisingly, the sequence of the fragment obtained using our primers was different from the sequence listed in the dbEST data base for clone R74593. In particular, our new sequence contained an additional guanosine base inserted 3′ to the putative stop codon, but 5′ to the segment encoding the Kunitz-like sequence (FIG. 3). The insertion of an additional G shifted the stop codon out of the reading frame for the Kunitz-like domain (G at base pair 114 of the corrected sequence for R74593; FIG. 3).

Subsequent query of the dbEST for sequences homologous to the Kunitz-like peptide sequence of R74593 yielded H94519 derived from human retina library and N39798. These sequences contained a Kunitz-like sequence that was almost identical to the Kunitz-like domain encoded in R35464 except that it contained all six of the characteristic cysteines. Overlay of each of the nucleotide sequences with that of R74593 (corrected by the insertion of G at b.p. 114) and R35464 was used to obtain a consensus nucleotide sequence for a partial human placental bikunin (SEQ ID NO.: 9; FIG. 3). The translated consensus sequence yielded an open reading frame extending from residue −18 to +179 (FIG. 3; full translation SEQ ID NO.: 10) that contained two complete Kunitz-like domain sequences, within the region of amino acid residues 17-64 and 102-159 respectively.

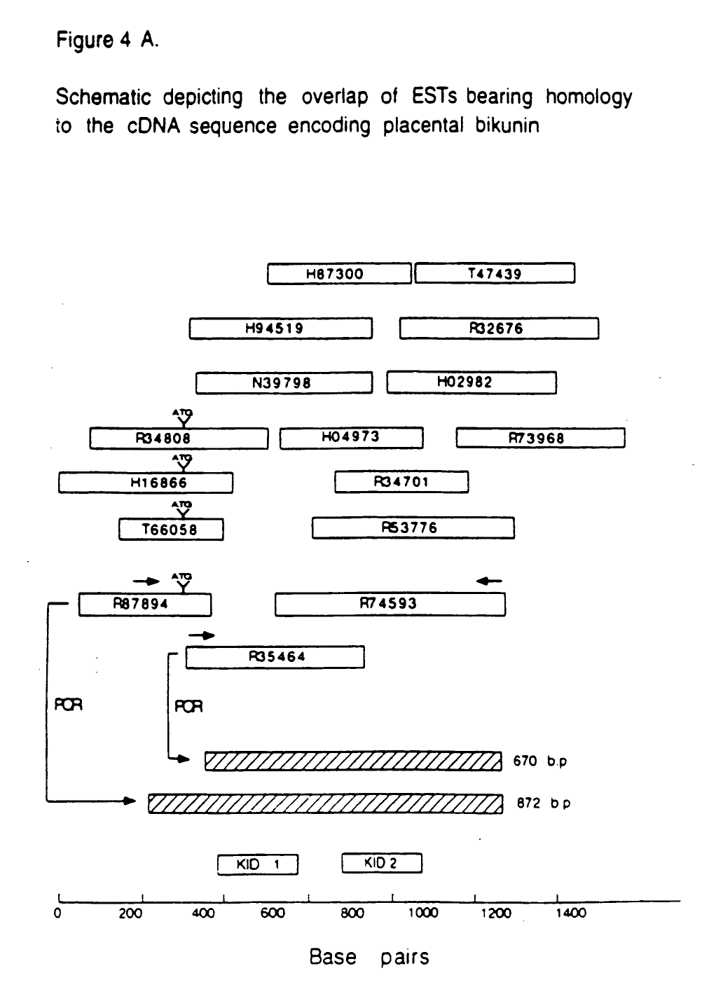

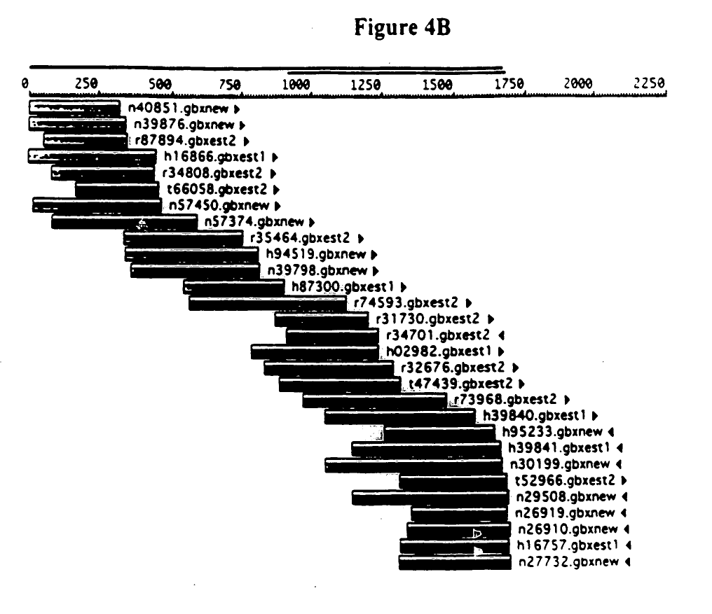

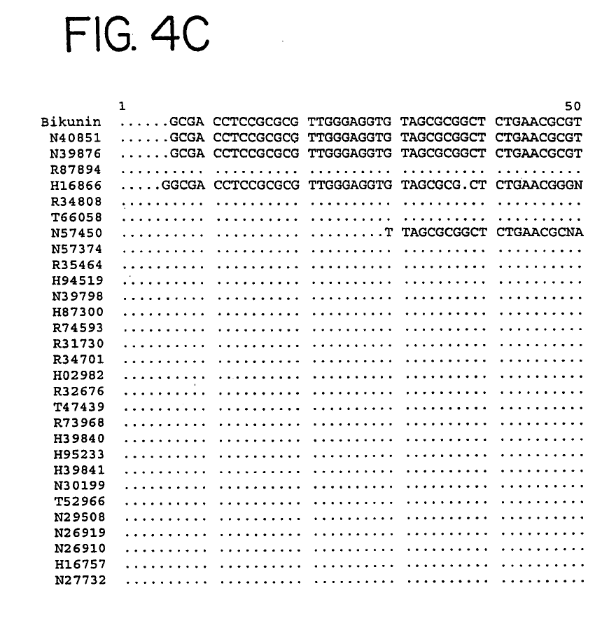

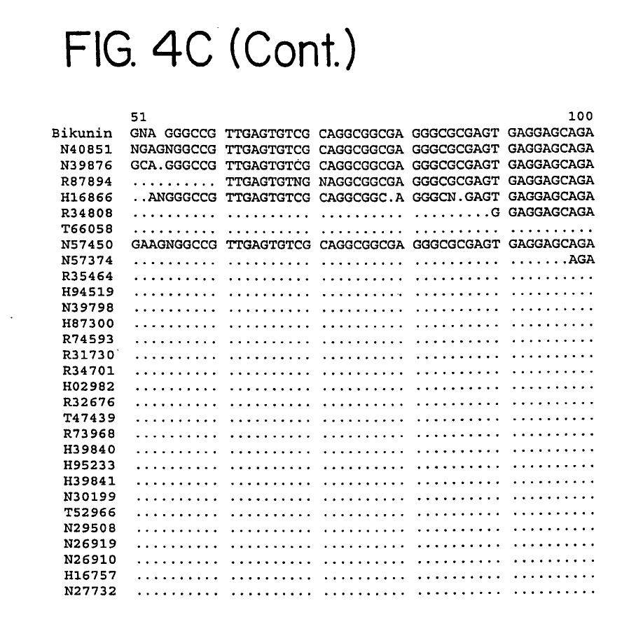

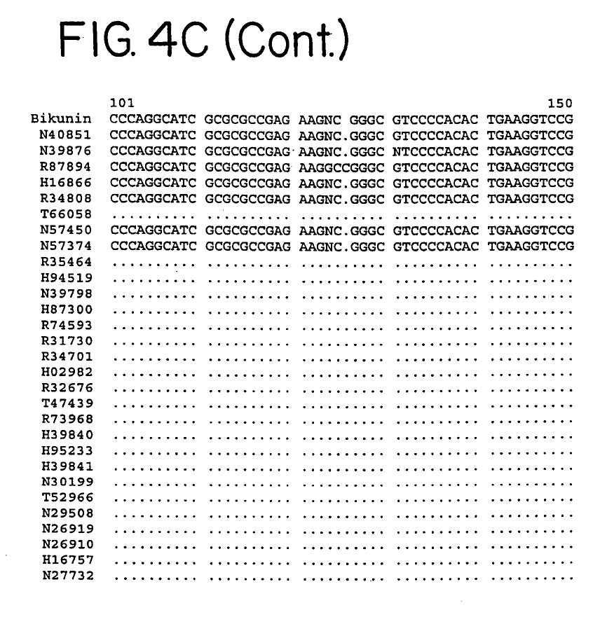

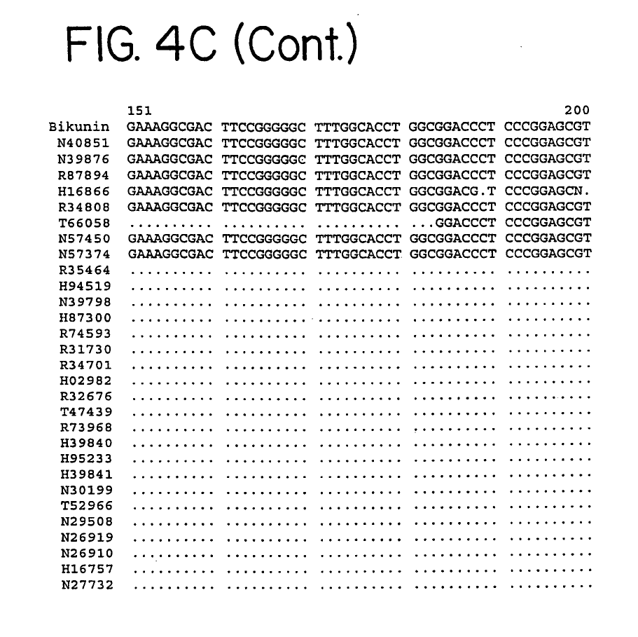

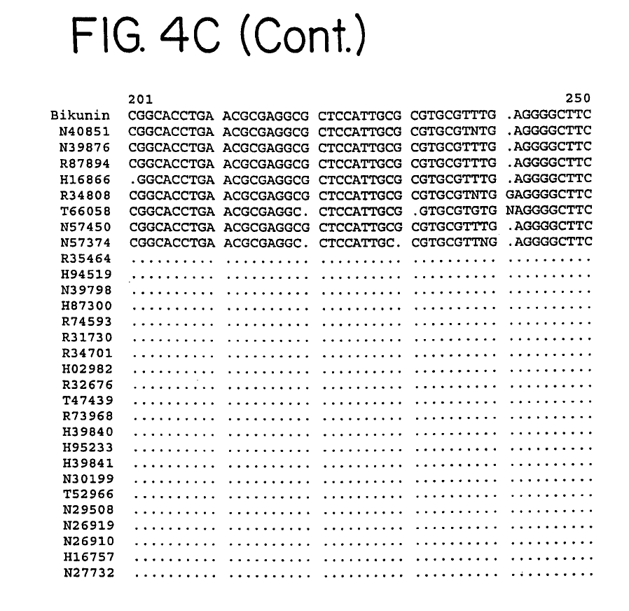

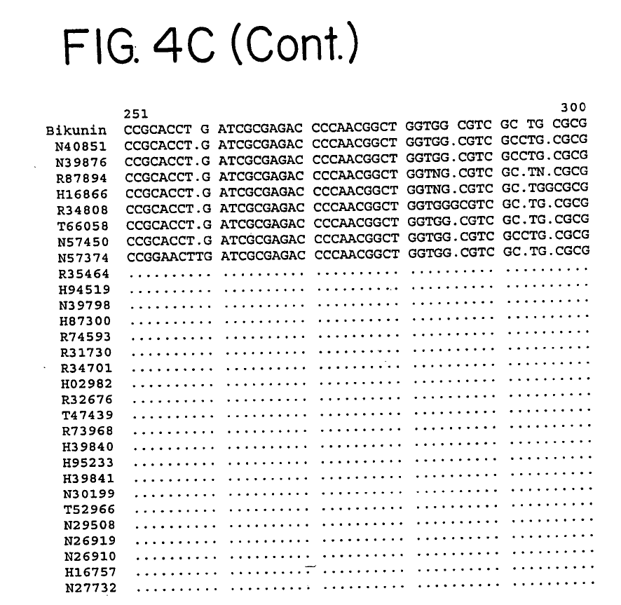

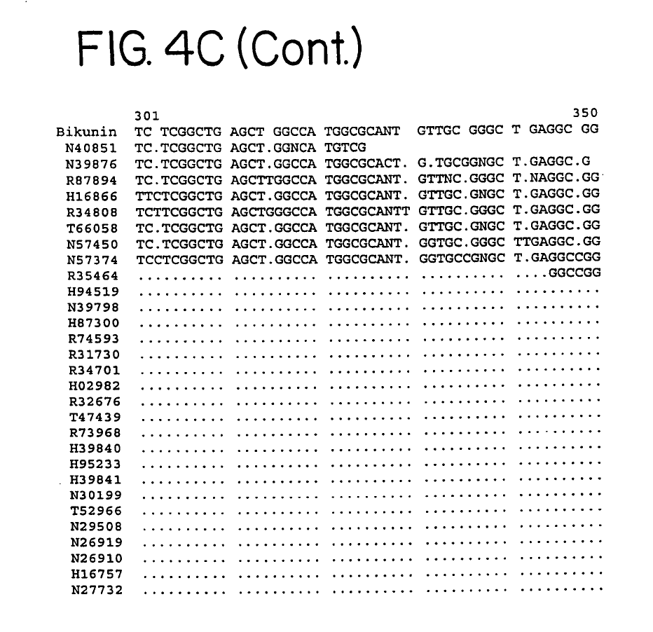

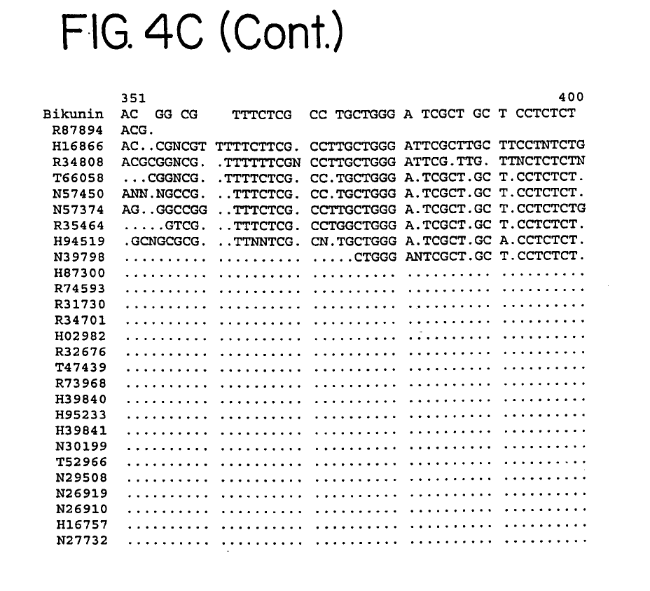

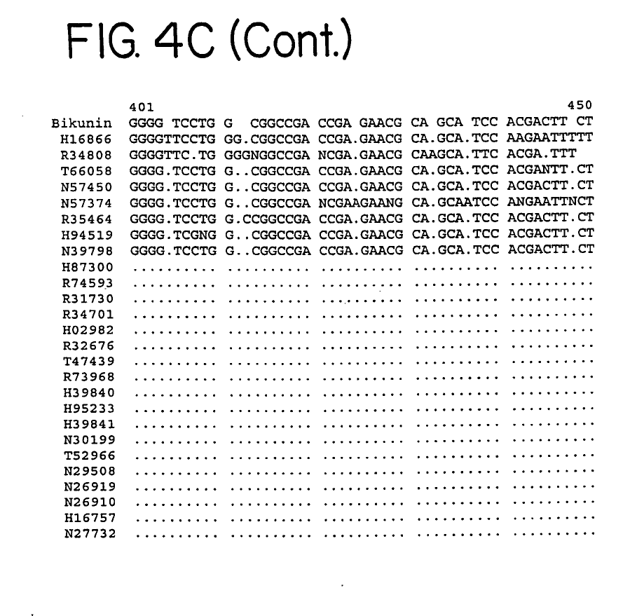

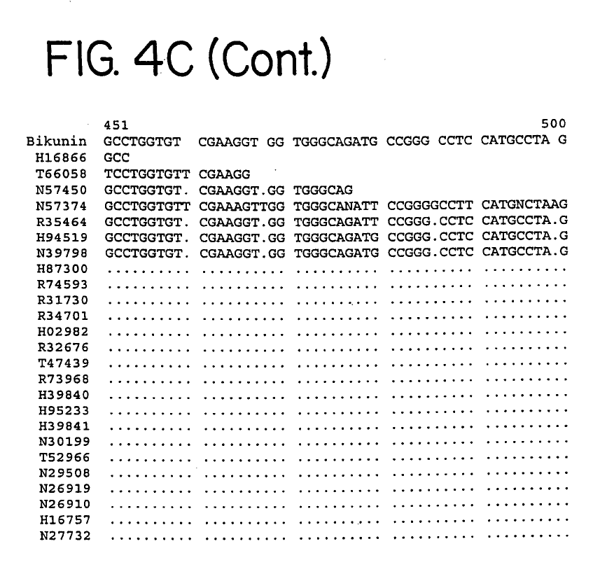

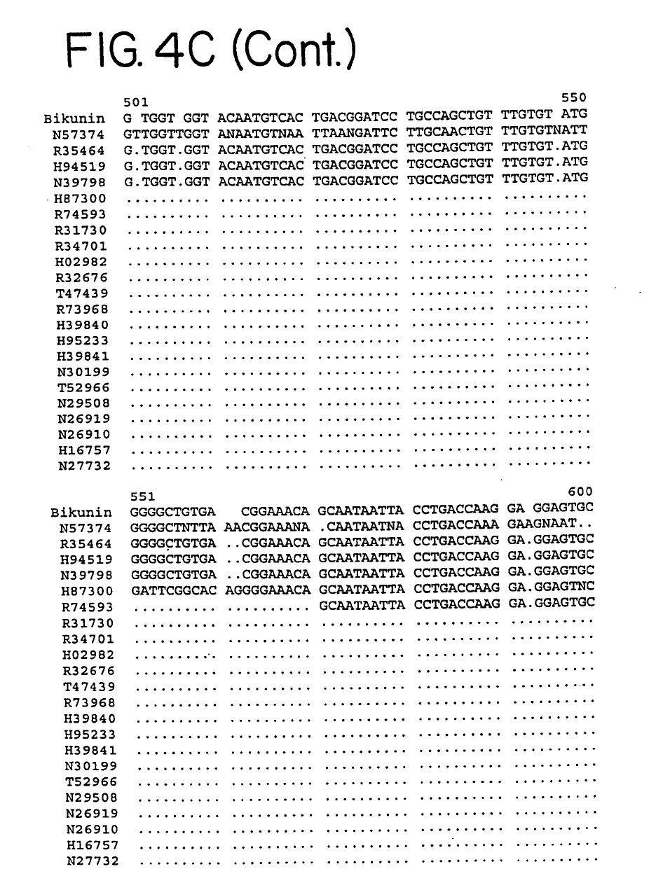

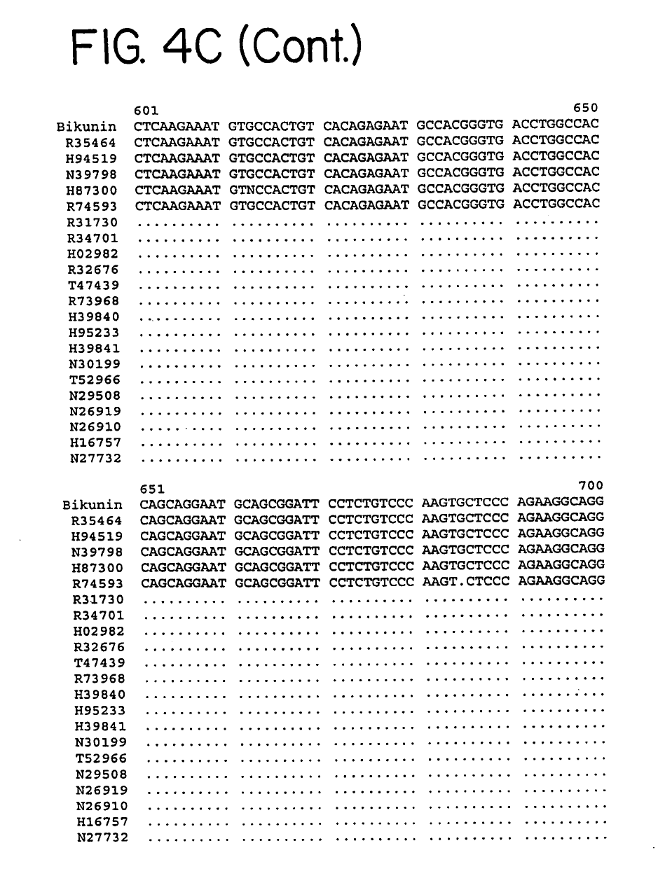

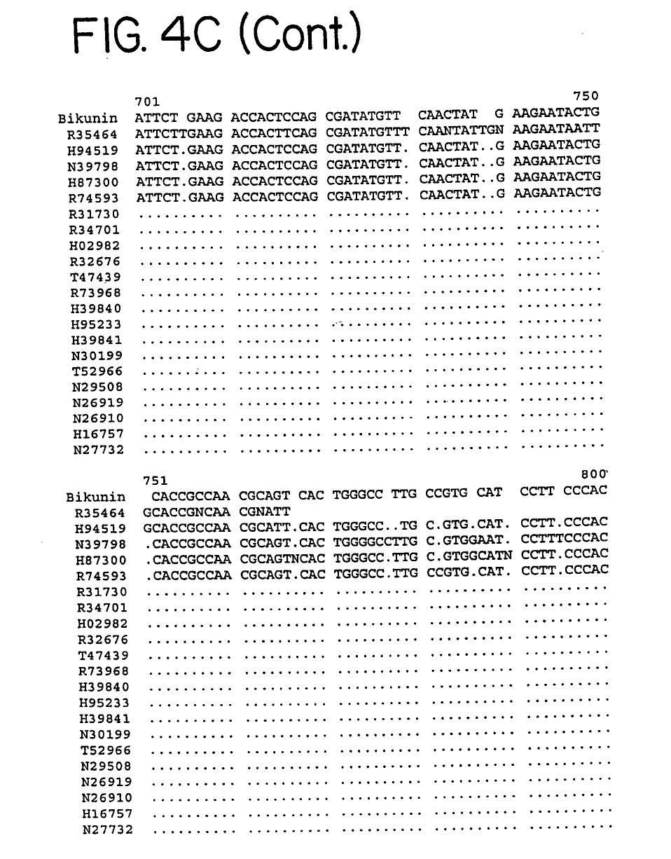

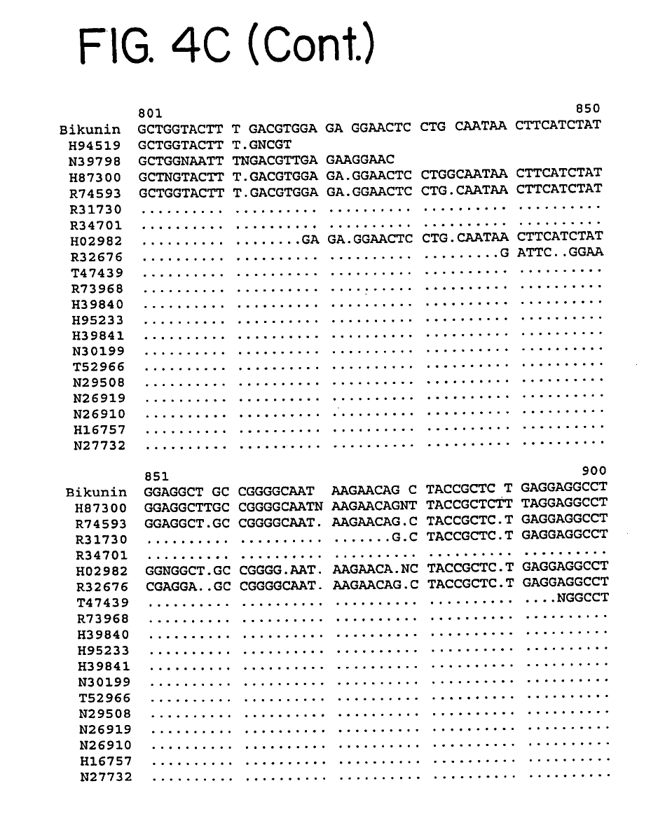

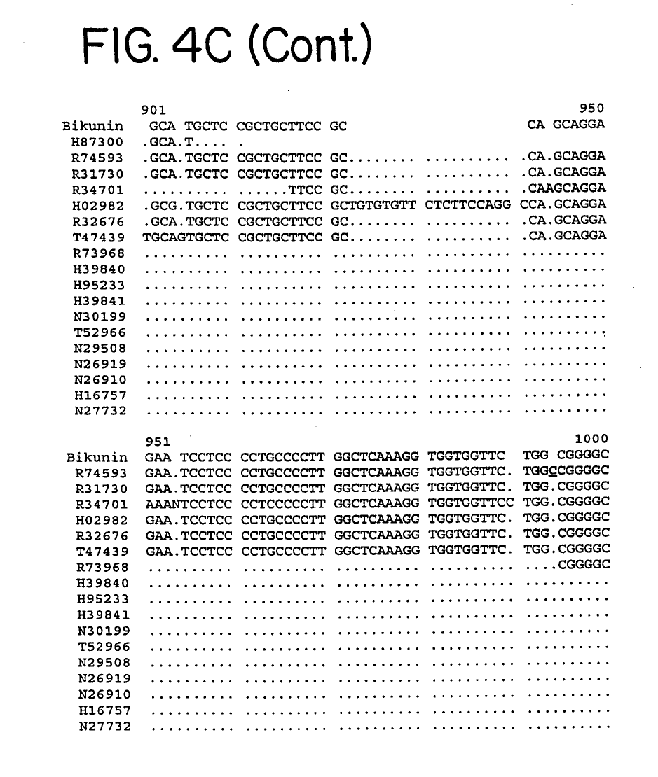

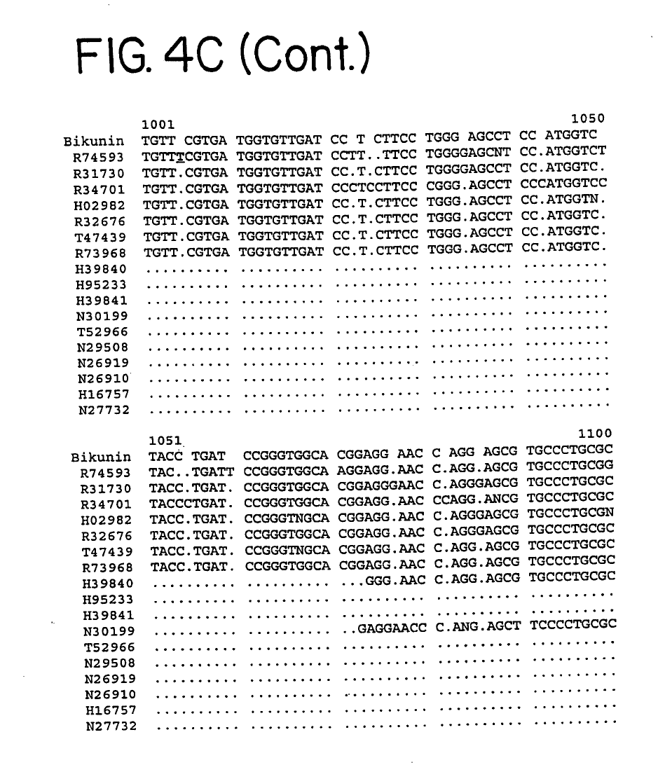

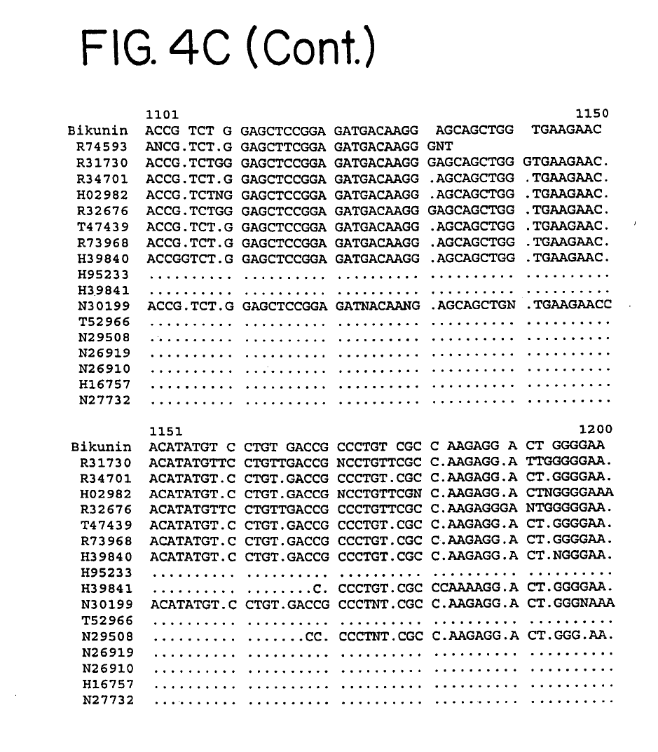

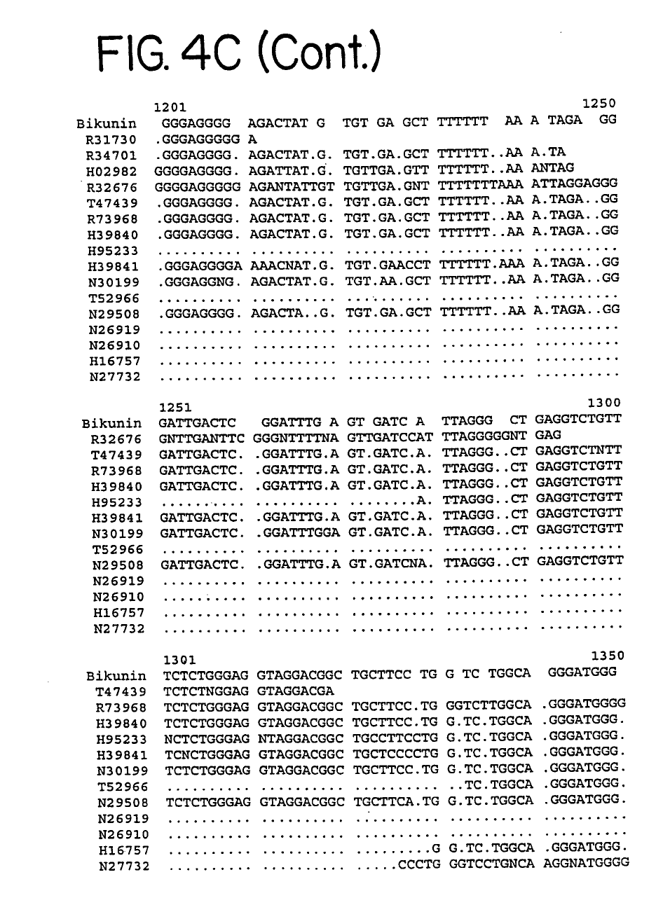

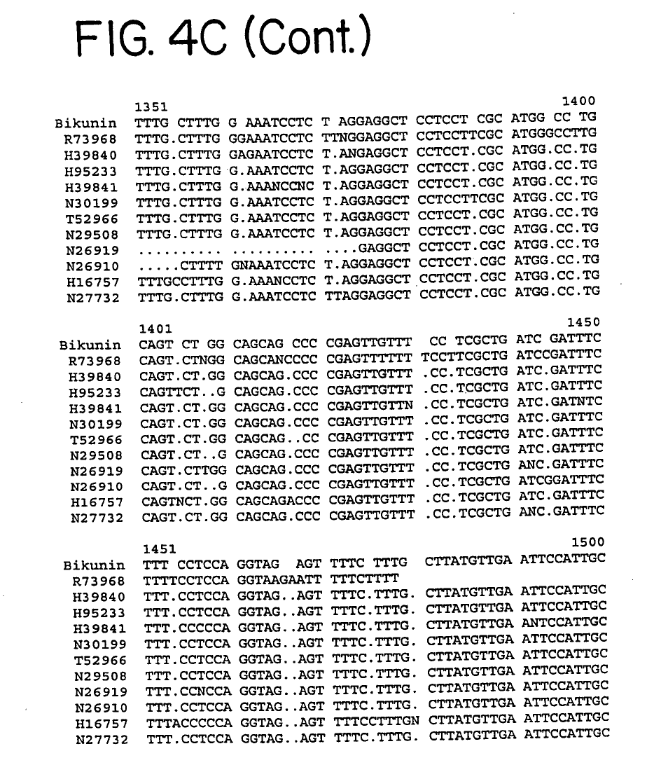

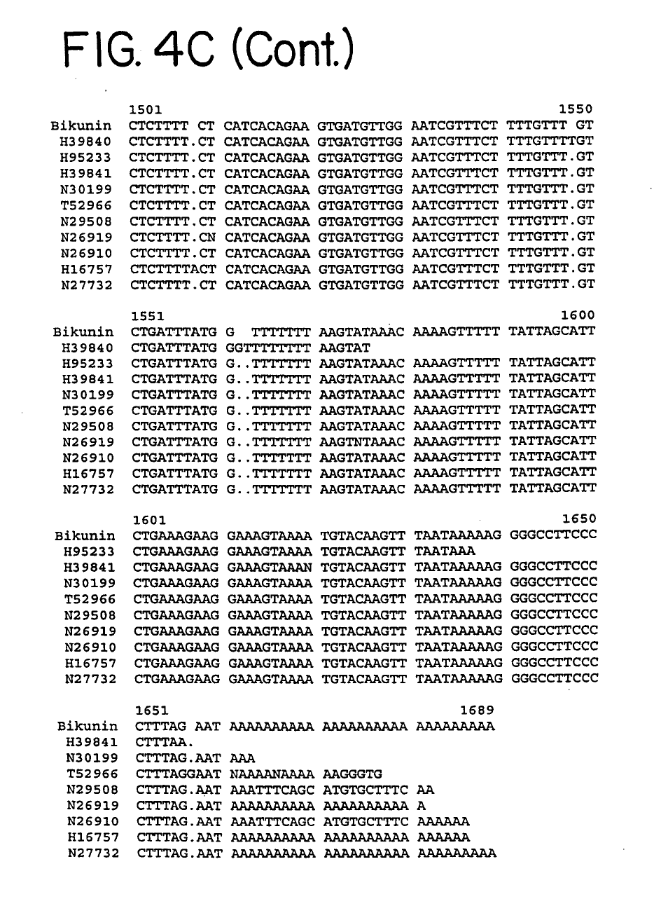

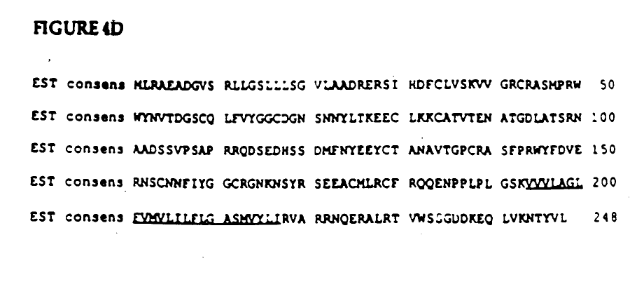

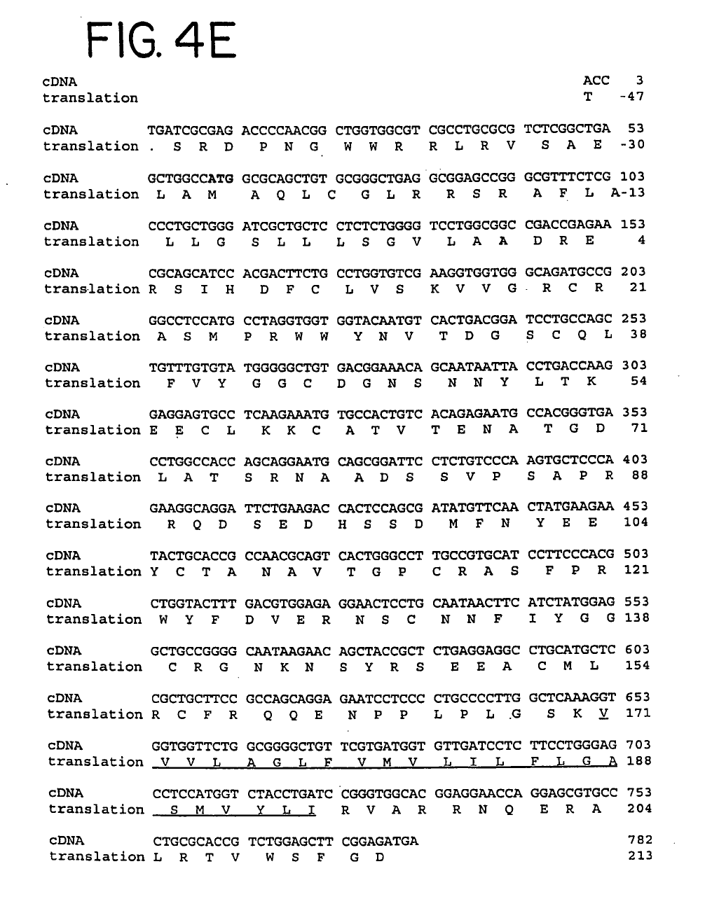

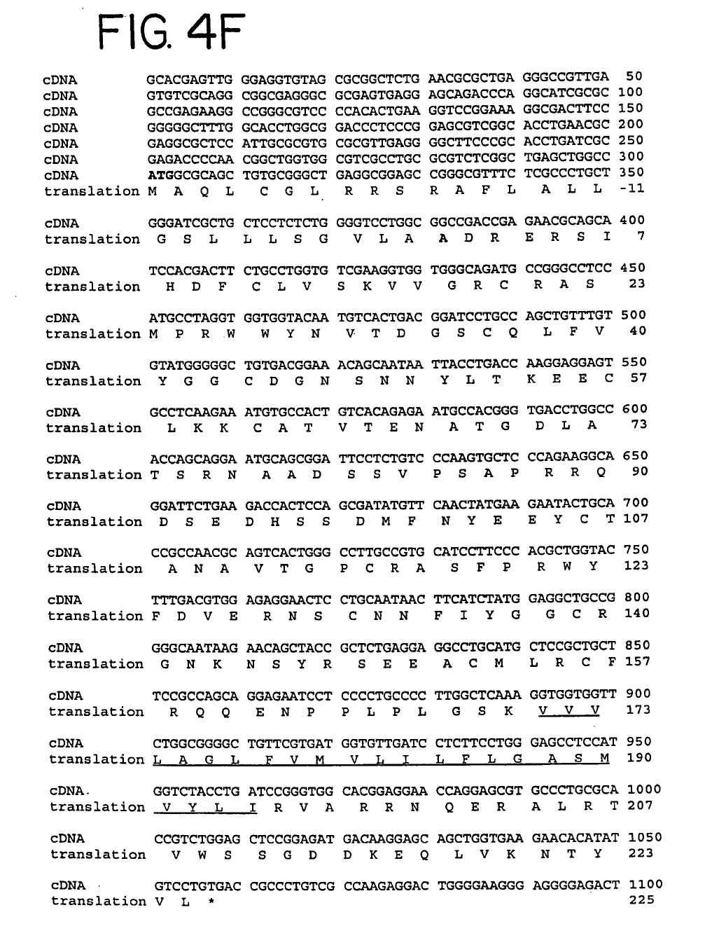

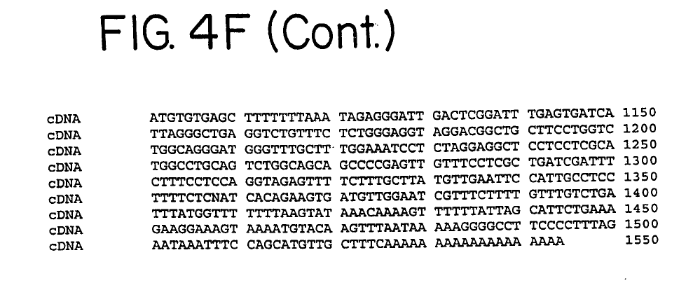

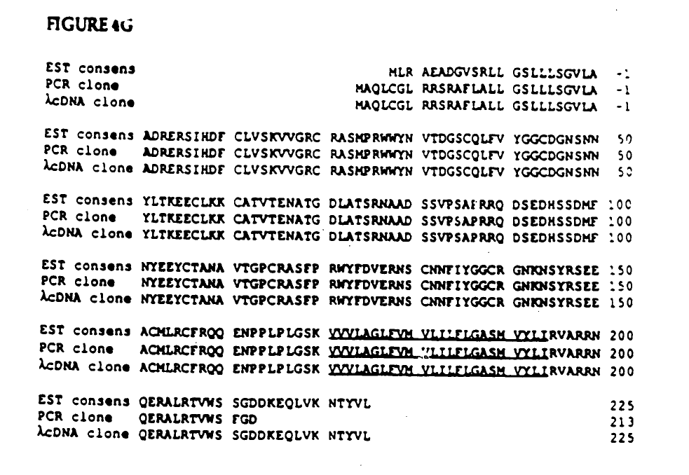

Further efforts attempted to obtain additional 5′ sequence by querying dbEST with the sequence of R35464. Possible matches from such searches, that possessed additional 5′ sequence were then in turn used to re-query the dbEST. In such an iterative fashion, a series of overlapping 5′ sequences were identified which included clones H16866, T66058, R34808, R87894, N40851 and N39876 (FIG. 4). Alignment of some of these sequences suggested the presence of a 5′ ATG which might serve as a start site for synthesis of the consensus translated protein sequence. From this selected information, it was now possible to selectively screen for, and determine the nucleic acid and polypeptide sequences of a human protein with homologous function to aprotinin.

Re-interrogation of the dbEST revealed a number of new EST entries shown schematically in FIG. 4B. Overlap with these additional ESTs allowed us to construct a much longer consensus oligonucleotide sequence (FIG. 4C) that extended both 5′ and 3′ beyond the original oligonucleotide sequence depicted in FIG. 3. In fact, the new sequence of total length 1.6 kilobases extended all the way to the 3′ poly-A tail. The increased number of overlapping ESTs at each base-pair position along the sequence improved the level of confidence in certain regions such as the sequence overlapping with the 3′ end of EST R74593 (FIG. 3). Several overlapping ESTs in this region corroborated two critical base deletions relative to R74593 (located as bold underlined in FIG. 4C, map positions 994 and 1005). Translation of the new consensus sequence (FIG. 4D) in the bikunin encoding frame yielded a form of placental bikunin that was larger (248 amino acids) than the mature sequence (179 amino acids) encoded from the original consensus (SEQ ID NO.: 1), and was terminated by an in-frame stop codon within the oligonucleotide consensus. The size increase was due to a frame shift in the 3′ coding region resulting from removal of the two base insertions unique to EST R74593. The frame shift moved the stop codon of the original consensus (FIG. 3) out of frame enabling read through into a new frame encoding the additional amino acid sequence. The new translation product (FIG. 4D) was identical to the original protein consensus sequence (SEQ ID NO.: 1) between residues +1 to +175 (encoding the Kunitz domains), but contained a new C-terminal extension exhibiting a putative 24 residue long transmembrane domain (underlined in FIG. 4D) followed by a short 31 residue cytoplasmic domain. The precise sequence around the initiator methionine and signal peptide was somewhat tentative due to considerable heterogeneity amongst the overlapping ESTs in this region.

Analysis of the protein sequence by Geneworks™, highlighted asparagine residues at positions 30 and 67 as consensus sites for putative N-linked glycosylation. Asparagine 30 was not observed during N-terminal sequencing of the full length protein isolated from human placenta, consistent with it being glycosylated.

### Cloning of Human Bikunin

The existence of a human mRNA corresponding to the putative human bikunin nucleotide sequence inferred from the analysis of FIG. 3, was confirmed as follows. The nucleic acid primer hybridizing 5′ to the Kunitz-encoding cDNA sequence of R35464 (b.p. 3-27 of consensus nucleotide sequence in FIG. 3): GGTCTAGAGGCCGGGTCGTTTCTCGCCTGGCTGGGA (a 5′ primer derived from R35464 sequence with an XbaI site; SEQ ID NO.: 35), and the nucleic acid primer hybridizing 3′ to the Kunitz encoding sequence of R74593 (b.p. 680-700 of consensus nucleotide sequence in FIG. 3), was used to PCR amplify, from a Clontech human placental library, a fragment of the size (ca. 670 b.p) expected from a cDNA consensus nucleotide sequence encoding the placental bikunin sequence of FIG. 3 (Shown schematically in FIG. 4A).

Using a 5′ primer hybridizing to a sequence in R87894 that is 126 b.p 5′ to the putative ATG start site discussed above, (shown schematically in FIG. 4A at b.p. 110) plus the same 3′ primer to R74593 as used above, it was possible to amplify a fragment from a Clontech human placental library of the expected size (approximately 872 b.p) predicted by EST overlay (Shown schematically in FIG. 4).

Sequencing of the 872 b.p. fragment showed it to contain nucleotide segment corresponding to b.p. 110 to 218 of EST R87894 at its 5′ end and b.p. 310 to 542 of the consensus sequence for placental bikunin inferred from the EST overlay analysis (of FIG. 3), at its 3′ end. This 3′ nucleotide sequence contained all of the Kunitz-like domain encoded by placental bikunin (102-159).

To obtain a cDNA encoding the entire extracellular region of the protein, the following 5′ PCR primer: CACCTGATCGCGAGACCCC (SEQ ID NO.: 36) designed to hybridize to a sequence within EST R34808 was used with the same 3′ primer to EST 74593 to amplify (30 cycles) an approximately 780 base-pair cDNA product from the human placental cDNA library. This product was gel purified, and cloned into the TA vector (Invitrogen) for DNA sequencing by the dideoxy method (Sanger F., et al., (1977) Proc. Natl. Acad. Sci (USA), 74, pp 5463-5467) with the following primers:

(SEQ ID NO.: 37)

| | |
|---|---|
| Vector Specific: | GATTTAGGTGACACTATAG (SP6) |

(SEQ ID NO.: 38)

TAATACGACTCACTATAGGG (T7)

(SEQ ID NO.: 39)

| | |
|---|---|
| Gene Specific: | TTACCTGACCAAGGAGGAGTGC |

(SEQ ID NO.: 40)

AATCCGCTGCATTCCTGCTGGTG

(SEQ ID NO.: 41)

CAGTCACTGGGCCTTGCCGT

The resulting cDNA sequence is depicted in FIG. 4E together with its translation product. At the nucleotide level, the sequence exhibited only minor differences from the consensus EST sequence (FIG. 4D). Translation of the sequence yielded a coding sequence containing an in-frame initiator ATG site, signal peptide and mature placental bikunin sequence and transmembrane domain. The translated sequence of the PCR product was missing the last 12 amino acid residues from the cytoplasmic domain as a consequence of the choice of selection of the 3′ primer for PCR amplification. This choice of 3′ PCR primer (designed based on the sequence of R74593) was also responsible for the introduction of an artifactual S to F mutation at amino acid position 211 of the translated PCR-derived sequence. The signal peptide deduced from translation of the PCR fragment was somewhat different to that of the EST consensus.

To obtain a full length placental bikunin cDNA, the PCR derived product (FIG. 4E) was gel purified and used to isolate a non-PCR based full length clone representing the bikunin sequence. The PCR derived cDNA sequence was labeled with ³²P-CTP by High Prime (Boehringer Mannheim) and used to probe a placental cDNA Library (Stratagene, Unizap™ λ library) using colony hybridization techniques. Approximately 2×10⁶ phage plaques underwent 3 rounds of screening and plaque purification. Two clones were deemed full length (˜1.5 kilobases) as determined by restriction enzyme analysis and based on comparison with the size of the EST consensus sequence (see above). Sequencing of one of these clone by the dideoxy method yielded the oligonucleotide sequence depicted in FIG. 4F. The translation product from this sequence yielded a protein with inframe initiator methionine, signal peptide and mature placental bikunin sequence. The mature placental bikunin sequence was identical to the sequence of the mature protein derived by translation of the EST consensus although the signal peptide sequence lengths and sequences differed. Unlike the PCR derived product, the cDNA derived by colony hybridization contained the entire ectodomain, transmembrane domain, cytoplasmic domain and in-frame stop codon. In fact, the clone extended all the way to the poly-A tail. The initiator methionine was followed by a hydrophobic signal peptide which was identical to the signal peptide encoded in the PCR derived clone. Subsequently, we expressed and purified soluble fragments of placental bikunin, Bikunin (1-170), from Sf9 cells (Example 9) and CHO cells (Example 17), and found them to be functional protease inhibitors (Examples 10 and 18). Furthermore, we isolated from human placenta a soluble fragment of placental bikunin which was also an active protease inhibitor (Example 7).

Based on the above observations, it seems that full length placental bikunin has the capacity to exist as a transmembrane protein on the surface of cells as well as a soluble protein. Other transmembrane proteins that contain Kunitz domains are known to undergo proteolytic processing to yield mixtures of soluble and membrane associated forms. These include two forms of the Amyloid Precursor Protein termed APP751 (Esch F., et al., (1990) Science, 248, pp 1122-1124) and APP 770 (Wang R., et al., (1991), J. Biol Chem, 266, pp 16960-16964).

Contact activation is a process which is activated by exposure of damaged vascular surfaces to components of the coagulation cascade. Angiogenesis is a process that involves local activation of plasmin at endothelial surfaces. The specificity of placental bikunin and its putative capacity to anchor to cell surfaces, suggest that the physiologic functions of transmembranous placental bikunin may include regulation of contact activation and angiogenesis.

The amino acid sequences for placental bikunin (7-64), bikunin (102-159), and full length placental bikunin (FIG. 4F) were searched against the PIR (Vers. 46.0) and PatchX (Vers. 46.0) protein databases as well as the GeneSeq (Vers. 20.0) protein database of patented sequences using the Genetics Computer Group program FastA. Using the Genetics Computer Group program TFastA (Pearson and Lipman, 1988, Proc. Natl. Acad. Sci. USA 85: 2444-2448), these same protein sequences were searched versus the six-frame translations of the GenBank (Vers. 92.0 with updates to Jan. 26, 1996) and EMBL (modified Vers. 45.0) nucleotide databases as well as the GeneSeq (Vers. 20.0) nucleotide database of patented sequences. The EST and STS subsets of GenBank and EMBL were not included in this set of searches. The best matches resulting from these searches contained sequences which were only about 50% identical over their full length to the 58-amino acid protein sequence derived from our analysis of clones R74593 and R35464.

### Isolation of Human Bikunin

As mentioned above, synthetic peptides corresponding to bikunin (7-64) and bikunin (102-159) as determined from the translated consensus sequence for bikunin (FIG. 3), could be refolded (Examples 2 and 1, respectively) to yield active kallikrein inhibitor protein (Example 4 and 3, respectively). We exploited this unexpected property to devise a purification scheme to isolate native placental bikunin from human tissue.

Using a purification scheme which employed kallikrein-sepharose affinity chromatography as a first step, highly purified native potent kallikrein inhibitor was isolated. The isolated native human bikunin had an identical N-terminus (sequenced for 50 amino acid residues) as the sequence predicted by the translation of the consensus nucleic acid sequence (FIG. 3) amino acid residues +1 to +50 (Example 7). This confirmed for the first time the existence of a novel native kallikrein inhibitor isolated from human placenta.

Known Kunitz-like domains are listed below. Residues believed to be making contact with target proteases are highlighted as of special interest (bold/underlined). These particular residues are named positions Xaa¹⁻⁶ for specific reference as shown by label Xaa.

Bikunin (7-64) (SEQ ID NO.: 4)
Xaa 1 1 111 1 1
1 2 3 456789 0 1 234 5 6
IHDFCLVSKVV GRCRASMPRW WYNVTDGSCQ LFVYGGCDGN SNNYLTKEEC LKKCATV
Bikunin (102-159) (SEQ ID NO.: 6)
YEEYCTANAVT GPCRASFPRW YFDVERNSCN NFIYGGCRGN KNSYRSEEAC MLRCFRQ
Tissue factor pathway inhibitor precursor 1 (SEQ ID NO.: 18)
-HSFCAFKADD GPCKAIMKRF FFNIFTRQCE EFIYGGCEGN QNRFESLEEC KKMCTRD
Tissue factor pathway inhibitor precursor 1 (SEQ ID NO.: 19)
-PDFCFLEEDP GICRGYITRY FYNNQTKQCE RFKYGGCLGN MNNFETLEEC KNICEDG
Tissue factor pathway inhibitor precursor (SEQ ID NO.: 20)
-PSWCLTPADR GLCRANENRF YYNSVIGKCR PFKYSGCGGN ENNFTSKQEC LRACKKG
Tissue factor pathway inhibitor precursor 2 (SEQ ID NO.: 21)
-AEICLLPLDY GPCRALLLRY YYRYRTQSCR QFLYGGCEGN ANNFYTWEAC DDACWRI
Tissue factor pathway inhibitor precursor 2 (SEQ ID NO.: 22)
-PSFCYSPKDE GLCSANVTRY YFNPRYRTCD AFTYTGCGGN DNNFVSREDC KRACAKA
Amyloid precursor protein homologue (SEQ ID NO: 23)
-KAVCSQEAMT GPCRAVMPRT TFDLSKGKCV RFITGGCGGN RNNFESEDYC MAVCKAM
Aprotinin (SEQ ID NO: 24)
RPDFCLEPPYT GPCKARIIRY FYNAKAGLCQ TFVYGGCRAK RNNFKSAEDC MRTCGGA
Inter-α-trypsin inhibitor precursor (SEQ ID NOs: 25)
----CQLGYSA GPCMGMTSRY FYNGTSMACE TFQYGGCMGN GNNFVTEKEC LQTC
Inter-α-trypsin inhibitor precursor (SEQ ID NOs: 26)
VAACNLPIVR GPCRAFIQLWAFDAVKGKCV LFPYGGCQGN GNKFYSEKEC REYCGVP
Amyloid precursor protein (SEQ ID NO: 27)
-EVCCSEQAET GPCRAMISRW YFDVTEGKCA PFFYGGCGGN RNNFDTEEYC MAVCGSA
Collagen α-3(VI) precursor (SEQ ID NO: 28)
----CKLPKDE GTCRDFILKW YYDPNTKSCA RFWYGGCGGN ENKFGSQKEC EKVC
HKI-B9 (SEQ ID NO: 29)
-PNVCAFPMEK GPCOTYMTRW FFNFETGECE LFAYGGCGGN SNNFLRKEKC EKFCKFT

The placental bikunin, isolated domains or other variants of the present invention may be produced by standard solid phase peptide synthesis using either t-Boc chemistry as described by Merrifield R. B. and Barany G., in: The peptides, Analysis, Synthesis, Biology, 2, Gross E. et al., Eds. Academic Press (1980) Chapter 1; or using F-moc chemistry as described by Carpino L. A., and Han G. Y., (1970) J. Amer Chem Soc., 92, 5748-5749, and illustrated in Example 2. Alternatively, expression of a DNA encoding the placental bikunin variant may be used to produce recombinant placental bikunin variants.

The instant invention provides for the use of a purified human serine protease inhibitor which can specifically inhibit kallikrein, that has been isolated from human placental tissue via affinity chromatography. The human serine protein inhibitor, herein called human placental bikunin, contains two serine protease inhibitor domains of the Kunitz class. In one particular embodiment, the instant invention embodies a protein having the amino acid sequence:

(SEQ ID NO.: 1)

| | |
|---|---|
| ADRERSIHDF CLVSKVVGRC RASMPRWWYN VTDGSCQLFV | 50 |
| YGGCDGNSNN | |
| YLTKEECLKK CATVTENATG DLATSRNAAD SSVPSAPRRQ | 100 |
| DSEDHSSDMF | |
| NYEEYCTANA VTGPCRASFP RWYFDVERNS CNNFIYGGCR | 150 |
| GNKNSYRSEE | |
| ACMLRCFRQQ ENPPLPLGSK VVVLAGAVS | 179 |

In a preferred embodiment the instant invention provides for a bikunin protein (Bikunin (1-170)) having the amino acid sequence:

(SEQ ID NO.: 52)

| | |
|---|---|
| ADRERSIHDF CLVSKVVGRC RASMPRWWYN VTDGSCQLFV | 50 |
| YGGCDGNSNN | |
| YLTKEECLKK CATVTENATG DLATSRNAAD SSVPSAPRRQ | 100 |
| DSEDHSSDMF | |
| NYEEYCTANA VTGPCRASFP RWYFDVERNS CNNFIYGGCR | 150 |
| GNKNSYRSEE | |
| ACMLRCFRQQ ENPPLPLGSK | 179 |

In one aspect, the biological activity of the protein useful in practicing the instant invention is that it can bind to and substantially inhibit the biological activity of trypsin, human plasma and tissue kallikreins, human plasmin and Factor XIIa. In a preferred embodiment, the present invention provides for a native human placental bikunin protein, in glycosylated form. In a further embodiment the instant invention encompasses native human bikunin protein which has been formed such that it contains at least one cysteine-cysteine disulfide bond. In a preferred embodiment, the protein contains at least one intra-chain cysteine-cysteine disulfide bond formed between a pair of cysteines selected from the group consisting of CYS11-CYS61, CYS20-CYS44, CYS36-CYS57, CYS106-CYS156, CYS115-CYS139, and CYS131-CYS152, wherein the cysteines are numbered according to the amino acid sequence of native human placental bikunin. One of ordinary skill will recognize that the protein of the instant invention may fold into the proper three-dimensional conformation, such that the biological activity of native human bikunin is maintained, where none, one or more, or all of the native intra-chain cysteine-cysteine disulfide bonds are present. In a most preferred embodiment, the protein of the instant invention is properly folded and is formed with all of the proper native cysteine-cysteine disulfide bonds.

Active protein for use in the instant invention can be obtained by purification from human tissue, such as placenta, or via synthetic protein chemistry techniques, as illustrated by the Examples below. It is also understood that the protein for use in the instant invention may be obtained using molecular biology techniques, where self-replicating vectors are capable of expressing the protein of the instant invention from transformed cells. Such protein can be made as non-secreted, or secreted forms from transformed cells. In order to facilitate secretion from transformed cells, to enhance the functional stability of the translated protein, or to aid folding of the bikunin protein, certain signal peptide sequences may be added to the NH2-terminal portion of the native human bikunin protein.

In one embodiment, the instant invention thus provides for the native human bikunin protein with at least a portion of the native signal peptide sequence intact. Thus one embodiment of the invention provides for native human bikunin with at least part of the signal peptide, having the amino acid sequence:

(SEQ ID NO.: 2)

| | | | | | |
|---|---|---|---|---|---|
| AGSFL | AWLGS | LLLSG | VLA | | −1 |
| ADRER | SIHDFC | CLVSK | VVGRC | RASMP | 25 |
| RWWYN | VTDGS | CQLFV | YGGCD | GNSNN | 50 |
| YLTKE | ECLKK | CATVT | ENATG | DLATS | 75 |
| RNAAD | SSVPS | APRRQ | DSEDH | SSDMF | 100 |
| NYEEY | CTANA | VTGPC | RASFP | RWYFD | 125 |
| VERNS | CNNFI | YGGCR | GNKNS | YRSEE | 150 |
| ACMLR | CFRQQ | ENPPL | PLGSK | VVVLA | 175 |
| GAVS | | | | | 179 |

In a preferred embodiment, the instant invention provides for the use of a native human placental bikunin protein with part of the leader sequence intact, having the amino acid sequence of SEQ ID NO.: 52 with an intact leader segment having the amino acid sequence:

| | | |
|---|---|---|
| MAQLCGL RRSRAFLALL GSLLLSGVLA | −1 | (SEQ ID NO.: 53) |

In another embodiment, the instant invention provides for the use of bikunin protein with part of the leader sequence intact, having the amino acid sequence of SEQ ID NO.: 52 with the intact leader segment having the amino acid sequence:

| | | |
|---|---|---|
| MLR AEADGVSRLL GSLLLSGVLA | −1 | (SEQ ID NO.: 54) |

In a preferred numbering system used herein the amino acid numbered +1 is assigned to the NH2-terminus of the amino acid sequence for native human placental bikunin. One will readily recognize that functional protein fragments can be derived from native human placental bikunin, which will maintain at least part of the biological activity of native human placental bikunin, and act as serine protease inhibitors.

In one embodiment, the protein for use in the method of the instant invention comprises a fragment of native human placental bikunin, which contains at least one functional Kunitz-like domain, having the amino acid sequence of native human placental bikunin amino acids 7-159, hereinafter called “bikunin (7-159)”. Thus the instant invention embodies a method that employs a protein having the amino acid sequence:

(SEQ ID NO.: 3)

| | | | | | |
|---|---|---|---|---|---|
| | IHDF | CLVSK | VVGRC | RASMP | 25 |
| RWWYN | VTDGS | CQLFV | YGGCD | GNSNN | 50 |
| YLTKE | ECLKK | CATVT | ENATG | DLATS | 75 |
| RNAAD | SSVPS | APRRQ | DSEDH | SSDMF | 100 |
| NYEEY | CTANA | VTGPC | RASFP | RWYFD | 125 |
| VERNS | CNNFI | YGGCR | GNKNS | YRSEE | 150 |
| ACMLR | CFRQ | | | | 159 |

where the amino acid numbering corresponds to that of the amino acid sequence of native human placental bikunin. Another functional variant of this embodiment can be the fragment of native human placental bikunin, which contains at least one functional Kunitz-like domain, having the amino acid sequence of native human placental bikunin amino acids 11-156, bikunin (11-156)

(SEQ ID NO.: 50)

| | | | | | |
|---|---|---|---|---|---|
| | | CLVSK | VVGRC | RASMP | 25 |
| RWWYN | VTDGS | CQLFV | YGGCD | GNSNN | 50 |
| YLTKE | ECLKK | CATVT | ENATG | DLATS | 75 |
| RNAAD | SSVPS | APRRQ | DSEDH | SSDMF | 100 |
| NYEEY | CTANA | VTGPC | RASFP | RWYFD | 125 |
| VERNS | CNNFI | YGGCR | GNKNS | YRSEE | 150 |
| ACMLR | C | | | | 156. |

One can recognize that the individual Kunitz-like domains are also fragments of the native placental bikunin. In particular, the instant invention contemplates the use of a protein having the amino acid sequence of a first Kunitz-like domain consisting of the amino acid sequence of native human placental bikunin amino acids 7-64, hereinafter called “bikunin (7-64)”. Thus in one embodiment the instant invention encompasses a protein which contains at least one Kunitz-like domain having the amino acid sequence:

(SEQ ID NO.: 4)

| | | | | | |
|---|---|---|---|---|---|
| | IHDF | CLVSK | VVGRC | RASMP | 25 |
| RWWYN | VTDGS | CQLFV | YGGCD | GNSNN | 50 |
| YLTKE | ECLKK | CATV | | | 64 |

where the amino acid numbering corresponds to that of the amino acid sequence of native human placental bikunin. Another form of the protein of the instant invention can be a first Kunitz-like domain consisting of the amino acid sequence of native human placental bikunin amino acids 11-61, “bikunin (11-61)” having the amino acid sequence:

(SEQ ID NO.: 5)

| | | | | | |
|---|---|---|---|---|---|
| | | CLVSK | VVGRC | RASMP | 25 |
| RWWYN | VTDGS | CQLFV | YGGCD | GNSNN | 50 |
| YLTKE | ECLKK | C | | | 61 |

The instant invention also provides for a protein having the amino acid sequence of a Kunitz-like domain consisting of the amino acid sequence of native human placental bikunin amino acids 102-159, hereinafter called “bikunin (102-159)”. Thus one embodiment the instant invention encompasses a protein which contains at least one Kunitz-like domain having the amino acid sequence:

(SEQ ID NO.: 6)

| | | | | | |
|---|---|---|---|---|---|
| YEEY | CTANA | VTGPC | RASFP | RWYFD | 125 |
| VERNS | CNNFI | YGGCR | GNKNS | YRSEE | 150 |
| ACMLR | CFRQ | | | | 159 |

where the amino acid numbering corresponds to that of the amino acid sequence of native human placental bikunin. Another form of this domain can be a Kunitz-like domain consisting of the amino acid sequence of native human placental bikunin amino acids 106-156, “bikunin (106-156)” having the amino acid sequence:

(SEQ ID NO.: 7)

| | | | | | |
|---|---|---|---|---|---|
| | CTANA | VTGPC | RASFP | RWYFD | 125 |
| VERNS | CNNFI | YGGCR | GNKNS | YRSEE | 150 |
| ACMLR | C | | | | 156 |

Thus one of ordinary skill will recognize that fragments of the native human bikunin protein can be made which will retain at least some of the native protein biological activity. Such fragments can also be combined in different orientations or multiple combinations to provide for alternative proteins which retain some of, the same, or more biological activity of the native human bikunin protein.

One will readily recognize that biologically active protein employed in the method of the instant invention may comprise one or more of the instant Kunitz-like domains in combination with additional Kunitz-like domains from other sources. Biologically active protein of the method of the instant invention may comprise one or more of the instant Kunitz-like domains in combination with additional protein domains from other sources with a variety of biological activities. The biological activity of the protein useful in practicing the instant invention can be combined with that of other known protein or proteins to provide for multifunctional fusion proteins having predictable biological activity. Thus, in one embodiment, the method of instant invention encompasses the use of a protein which contains at least one amino acid sequence segment the same as, or functionally equivalent to the amino acid sequence of either SEQ ID NO.: or SEQ ID NO.: 7.

An open reading frame which terminates at an early stop codon can still code for a functional protein. The instant invention encompasses such alternative termination, and in one embodiment provides for the use of a protein of the amino acid sequence:

(SEQ ID NO.: 8)

| | | | | | |
|---|---|---|---|---|---|
| ADRER | SIHDF | CLVSK | VVGRC | RASMP | 25 |
| RWWYN | VTDGS | CQLFV | YGGCD | GNSNN | 50 |
| YLTKE | ECLKK | CATVT | ENATG | DLATS | 75 |
| RNAAD | SSVPS | APRRQ | DS | | 92 |

In one embodiment, the instant invention provides for the use of substantially purified, or recombinantly produced native human bikunin protein with an intact segment of the leader sequence, and at least a portion of the native transmembrane region intact. Thus one embodiment of the invention provides for the use of native human bikunin, with an intact leader sequence, and with at least part of the transmembrane domain (underlined), having an amino acid sequence selected from:

| | | | | | | |
|---|---|---|---|---|---|---|
| EST | | | MLR | AEADGVSRLL | GSLLLSGVLA | −1 |
| PCR | | | MAQLCGL | RRSRAFLALL | GSLLLSGVLA | −1 |
| λcDNA | | | MAQLCGL | RRSRAFLALL | GSLLLSGVLA | −1 |
| EST | ADRERSIHDF | CLVSKVVGRC | RASMPRWWYN | VTDGSCQLFV | YGGCDGNSNN | 50 |
| PCR | ADRERSIHDF | CLVSKVVGRC | RASMPRWWYN | VTDGSCQLFV | YGGCDGNSN | 50 |
| λcDNA | ADRERSIHDF | CLVSKVVGRC | RASMPRWWYN | VTDGSCQLFV | YGGCDGNSN | 50 |
| EST | YLTKEECLKK | CATVTENATG | DLATSRNAAD | SSVPSAPRRQ | DSEDHSSDMF | 100 |
| PCR | YLTKEECLKK | CATVTENATG | DLATSRNAAD | SSVPSAPRRQ | DSEDHSSDMF | 100 |
| λcDNA | YLTKEECLKK | CATVTENATG | DLATSRNAAD | SSVPSAPRRQ | DSEDHSSDMF | 100 |
| EST | NYEEYCTANA | VTGPCRASFP | RWYFDVERNS | CNNFIYGGCR | GNKNSYRSEE | 150 |
| PCR | NYEEYCTANA | VTGPCRASFP | RWYFDVERNS | CNNFIYGGCR | GNKNSYRSEE | 150 |
| λcDNA | NYEEYCTANA | VTGPCRASFP | RWYFDVERNS | CNNFIYGGCR | GNKNSYRSEE | 150 |
| EST | ACMLRCFRQQ | ENPPLPLGSK | VVVLAGLFVM | VLILFLGASM | VYLIRVARRN | 200 |
| PCR | ACMLRCFRQQ | ENPPLPLGSK | VVVLAGLFVM | VLILFLGASM | VYLIRVARRN | 200 |
| λcDNA | ACMLRCFRQQ | ENPPLPLGSK | VVVLAGLFVM | VLILFLGASM | VYLIRVARRN | 200 |
| EST | QERALRTVWS | SGDDKEQLVK | NTYVL | | | 225 |
| PCR | QERALRTVWS | FGD | | | | 213 |
| λcDNA | QERALRTVWS | SGDDKEQLVK | NTYVL | | | 225 |

where EST is EST derived consensus SEQ ID NO.: 45, PCR is PCR clone SEQ ID NO.: 47, and λDNA is lambda cDNA clone SEQ ID NO.: 49. In a preferred embodiment a protein of the method of the instant invention comprises one of the amino acid sequence of SEQ ID NO.: 45, 47 or 49 wherein the protein has been cleaved in the region between the end of the last Kunitz domain and the transmembrane region (underlined).

The instant invention also embodies the use of the protein wherein the signal peptide is deleted. Thus the method of the instant invention provides for a protein having the amino acid sequence of SEQ ID NO.: 52 continuous with a transmembrane amino acid sequence:

(SEQ ID NO.: 69)

a transmembrane amino acid sequence:

(SEQ ID NO.: 68)

| | | | | |
|---|---|---|---|---|
| PCR | VVVLAGLFVM | VLILFLGASM | VYLIRVARRN | 200 |
| PCR | QERALRTVWS | FGD | | 213 |

or a transmembrane amino acid sequence:

(SEQ ID NO.: 67)

| | | | | |
|---|---|---|---|---|
| cDNA | VVVLAGLFVM | VLILFLGASM | VYLIRVARRN | 200 |
| λcDNA | QERALRTVWS | SGDDKEQLVK | NTYVL | 225. |

The protein amino acid sequences for use in the instant invention clearly teach one skilled in the art the appropriate nucleic acid sequences which can be used in molecular biology techniques to produce the proteins for use in the instant invention. Thus, one embodiment of the instant invention provides for use of a nucleic acid sequence which encodes for a human bikunin having the consensus DNA sequence of FIG. 3 (SEQ ID NO.: 9), which translates into the amino acid sequence for native human placental bikunin sequence of FIG. 3 (SEQ ID NO.: 10). In another embodiment, the instant invention provides for a consensus nucleic acid sequence of FIG. 4C (SEQ ID NO.: 51) which encodes for an amino acid sequence of FIG. 4D (SEQ ID NO.: 45).

In a preferred embodiment, the instant invention provides for the use of a nucleic acid sequence which encodes for native human placental bikunin having the DNA sequence of FIG. 4F (SEQ ID NO.: 48) which encodes for the protein sequence of SEQ ID NO.: 49. In an another embodiment, the instant invention provides for a nucleic acid sequence of FIG. 4E (SEQ ID NO.: 46) which encodes for a protein sequence of SEQ ID NO.: 47.

One can easily recognize that certain allelic mutations, and conservative substitutions made in the nucleic acid sequence can be made which will still result in a protein amino acid sequence encompassed by the method of the instant invention. One of skill in the art can recognize that certain natural allelic mutations of the protein of the instant invention, and conservative substitutions of amino acids in the protein of the instant invention will not significantly alter the biological activity of the protein, and are encompassed by the instant invention.

The instant invention also provides for pharmaceutical compositions containing human placental bikunin and fragments thereof that are useful for stimulating MCC in patients impaired by mucociliary dysfunction.

The present invention also provides methods for stimulating MCC in a patient suffering from mucociliary dysfunction, wherein an effective amount of the disclosed human serine protease inhibitors of the present invention in a biologically compatible vehicle is administered to the patient.

The present invention also provides for a method for stimulating MCC that employs variants of placental bikunin, and the specific Kunitz domains described above, that contain amino acid substitutions that alter the protease specificity. Preferred sites of substitution are indicated below as positions Xaa¹ through Xaa³² in the amino acid sequence for native placental bikunin. Substitutions at Xaa¹ through Xaa¹⁶ are also preferred for variants of bikunin (7-64), while substitutions at Xaa¹⁷ through Xaa³² are preferred for variants of bikunin (102-159).

Thus the method of the present invention embodies the use of a protein having an amino acid sequence:

(SEQ ID NO: 11)

| | |
|---|---|
| Ala Asp Arg Glu Arg Ser Ile Xaa¹ Asp Phe | 10 |
| Cys Leu Val Ser Lys Val Xaa² Gly Xaa³ Cys | 20 |
| Xaa⁴ Xaa⁵ Xaa⁶ Xaa⁷ Xaa⁸ Xaa⁹ Trp Trp Tyr Asn | 30 |
| Val Thr Asp Gly Ser Cys Gln Leu Phe Xaa¹⁰ | 40 |
| Tyr Xaa¹¹ Gly Cys Xaa¹² Xaa¹³ Xaa¹⁴ Ser Asn Asn | 50 |
| Tyr Xaa¹⁵ Thr Lys Glu Glu Cys Leu Lys Lys | 60 |
| Cys Ala Thr Xaa¹⁶ Thr Glu Asn Ala Thr Gly | 70 |
| Asp Leu Ser Thr Ser Arg Asn Ala Ala Asp | 80 |
| Ser Ser Val Pro Ser Ala Pro Arg Arg Gln | 90 |
| Asp Ser Glu His Asp Ser Ser Asp Met Phe | 100 |
| Asn Tyr Xaa¹⁷ Glu Tyr Cys Thr Ala Asn Ala | 110 |
| Val Xaa¹⁸ Gly Xaa¹⁹ Cys Xaa²⁰ Xaa²¹ Xaa²² Xaa²³ | 120 |
| Xaa²⁴ | |
| Xaa²⁵ Trp Tyr Phe Asp Val Glu Arg Asn Ser | 130 |
| Cys Asn Asn Phe Xaa²⁶ Tyr Xaa²⁷ Gly Cys Xaa²⁸ | 140 |
| Xaa²⁹ Xaa³⁰ Lys Asn Ser Tyr Xaa³¹ Ser Glu Glu | 150 |
| Ala Cys Met Leu Arg Cys Phe Arg Xaa³² Gln | 160 |
| Glu Asn Pro Pro Leu Pro Leu Gly Ser Lys | 170 |
| Val Val Val Leu Ala Gly Ala Val Ser | 179 |

where Xaa¹-Xaa³² each independently represents a naturally occurring amino acid residue except Cys, with the proviso that at least one of the amino acid residues Xaa¹-Xaa³² is different from the corresponding amino acid residue of the native sequence.

In the present context, the term “naturally occurring amino acid residue” is intended to indicate any one of the 20 commonly occurring amino acids, i.e., Ala, Arg, Asn, Asp, Cys, Gin, Glu, Gly, His, Ile, Leu, Lys, Met, Phe, Pro, Ser, Thr, Trp, Tyr and Val.

By substituting one or more amino acids in one or more of the positions indicated above, it may be possible to change the inhibitor specificity profile of native placental bikunin or that of the individual Kunitz-like domains, bikunin (7-64) or bikunin (102-159) so that it preferentially inhibits other serine proteases such as, but not limited to, the enzymes of the complement cascade, TF/FVIIa, FXa, prostasin, thrombin, neutrophil elastase, cathepsin G or proteinase-3.

Examples of preferred variants of placental bikunin include those wherein Xaa1 is an amino acid residue selected from the group consisting of His, Glu, Pro, Ala, Val or Lys, in particular wherein Xaa¹ is His or Pro; or wherein Xaa² is an amino acid residue selected from the group consisting of Val, Thr, Asp, Pro, Arg, Tyr, Glu, Ala, Lys, in particular wherein Xaa² is Val or Thr; or wherein Xaa³ is an amino acid residue selected from the group consisting of Arg, Pro, Ile, Leu, Thr, in particular wherein Xaa³ is Arg or Pro; or wherein Xaa⁴ is an amino acid residue selected from the group consisting of Arg, Lys and Ser, Gin, in particular wherein Xaa⁴ is Arg or Lys; or wherein Xaa⁵ is an amino acid residue selected from the group consisting of Ala, Gly, Asp, Thr, in particular wherein Xaa⁵ is Ala; or wherein Xaa⁶ is an amino acid residue selected from the group consisting of Ser, Ile, Tyr, Asn, Leu, Val, Arg, Phe, in particular wherein Xaa⁶ is Ser or Arg; or wherein Xaa⁷ is an amino acid residue selected from the group consisting of Met, Phe, Ile, Glu, Leu, Thr and Val, in particular wherein Xaa⁷ is Met or Ile; or wherein Xaa⁸ is an amino acid residue selected from the group consisting of Pro, Lys, Thr, Gin, Asn, Leu, Ser or Ile, in particular wherein Xaa⁸ is Pro or Ile; or wherein Xaa⁹ is an amino acid residue selected from the group consisting of Arg, Lys or Leu, in particular wherein Xaa⁹ is Arg: or wherein Xaa¹⁰ is an amino acid residue selected from the group consisting of Val, Ile, Lys, Ala, Pro, Phe, Trp, Gin, Leu and Thr, in particular wherein Xaa¹⁰ is Val; or wherein Xaa11 is an amino acid residue selected from the group consisting of Gly, Ser and Thr, in particular wherein Xaa¹¹ is Gly; or wherein Xaa¹² is an amino acid residue selected from the group consisting of Asp, Arg, Glu, Leu, Gln, Gly, in particular wherein Xaa¹² is Asp or Arg; or wherein Xaa¹³ is an amino acid residue selected from the group consisting of Gly and Ala; or wherein Xaa¹⁴ is an amino acid residue selected from the group consisting of Asn or Lys; or wherein Xaa¹⁵ is an amino acid residue selected from the group consisting of Gly, Asp, Leu, Arg, Glu, Thr, Tyr, Val, and Lys, in particular wherein Xaa¹⁵ is Leu or Lys; or wherein Xaa¹⁶ is an amino acid residue selected from the group consisting of Val, Gin, Asp, Gly, Ile, Ala, Met, and Val, in particular wherein Xaa¹⁶ is Val or Ala; or wherein Xaa¹⁷ is an amino acid residue selected from the group consisting of His, Glu, Pro, Ala, Lys and Val, in particular wherein Xaa¹⁷ is Glu or Pro; or wherein Xaa¹⁸ is an amino acid residue selected from the group consisting of Val, Thr, Asp, Pro, Arg, Tyr, Glu, Ala or Lys, in particular wherein Xaa¹⁸ is Thr; or wherein Xaa¹⁹ is an amino acid residue selected from the group consisting of Arg, Pro, Ile, Leu or Thr, in particular wherein Xaa¹⁹ is Pro; or wherein Xaa²⁰ is an amino acid residue selected from the group consisting of Arg, Lys, Gln and Ser, in particular wherein Xaa²⁰ is Arg or Lys; or wherein Xaa²¹ is an amino acid residue selected from the group consisting of Ala, Asp, Thr or Gly; in particular wherein Xaa²¹ is Ala; or wherein Xaa²² is an amino acid residue selected from the group consisting of Ser, Ile, Tyr, Asn, Leu, Val, Arg or Phe, in particular wherein Xaa²² is Ser or Arg; or wherein Xaa²³ is an amino acid residue selected from the group consisting of Met, Phe, Ile, Glu, Leu, Thr and Val, in particular wherein Xaa²³ is Phe or Ile; or wherein Xaa²⁴ is an amino acid residue selected from the group consisting of Pro, Lys, Thr, Asn, Leu, Gin, Ser or Ile, in particular wherein Xaa²⁴ is Pro or lie; or wherein Xaa²⁵ is an amino acid residue selected from the group consisting of Arg, Lys or Leu, in particular wherein Xaa²⁵ is Arg: or wherein Xaa²⁶ is an amino acid residue selected from the group consisting of Val, Ile, Lys, Leu, Ala, Pro, Phe, Gln, Trp and Thr, in particular wherein Xaa²⁶ is Val or Ile; or wherein Xaa²⁷ is an amino acid residue selected from the group consisting of Gly, Ser and Thr, in particular wherein Xaa²⁷ is Gly; or wherein Xaa²⁸ is an amino acid residue selected from the group consisting of Asp, Arg, Glu, Leu, Gly or Gin, in particular wherein Xaa²⁸ is Arg; or wherein Xaa²⁹ is an amino acid residue selected from the group consisting of Gly and Ala; or wherein Xaa³⁰ is an amino acid residue selected from the group consisting of Asn or Lys; or wherein Xaa³¹ is an amino acid residue selected from the group consisting of Gly, Asp, Leu, Arg, Glu, Thr, Tyr, Val, and Lys, in particular wherein Xaa³¹ is Arg or Lys; or wherein Xaa³² is an amino acid residue selected from the group consisting of Val, Gin, Asp, Gly, Ile, Ala, Met, and Thr, in particular wherein Xaa³² is Gln or Ala.

The invention also relates to DNA constructs that encode the Placental bikunin protein variants of the present invention. These constructs may be prepared by synthetic methods such as those described in Beaucage S. L. and Caruthers M. H., (1981) Tetrahedron Lett, 22, pp 1859-1862; Matteucci M. D and Caruthers M. H., (1981), J. Am. Chem. Soc. 103, p 3185; or from genomic or cDNA which may have been obtained by screening genomic or cDNA libraries with cDNA probes designed to hybridize with placental bikunin encoding DNA sequence. Genomic or cDNA sequence can be modified at one or more sites to obtain cDNA encoding any of the amino acid substitutions or deletions described in this disclosure.

The instant invention also relates to expression vectors containing the DNA constructs encoding the placental bikunin, isolated domains or other variants of the present invention that can be used for the production of recombinant placental bikunin variants. The cDNA should be connected to a suitable promoter sequence which shows transcriptional activity in the host cell of choice, possess a suitable terminator and a poly-adenylation signal. The cDNA encoding the placental bikunin variant can be fused to a 5′ signal peptide that will result in the protein encoded by the cDNA to undergo secretion. The signal peptide can be one that is recognized by the host organism. In the case of a mammalian host cell, the signal peptide can also be the natural signal peptide present in full length placental bikunin. The procedures used to prepare such vectors for expression of placental bikunin variants are well known in the art and are for example described in Sambrook et al., Molecular Cloning: A laboratory Manual, Cold Spring Harbor, N.Y., (1989).

The instant invention also relates to transformed cells containing the DNA constructs encoding the placental bikunin, isolated domains or other variants of the present invention that can be used for the production of recombinant placental bikunin variants. A variety of combinations of expression vector and host organism exist which can be used for the production of the placental bikunin variants. Suitable host cells include baculovirus infected Sf9 insect cells, mammalian cells such as BHK, CHO, Hela and C-127, bacteria such as E. coli, and yeasts such as Saccharomyces cervisiae. Methods for the use of mammalian, insect and microbial expressions systems needed to achieve expression of placental bikunin are well known in the art and are described, for example, in Ausubel F. M et al., Current Protocols in Molecular Biology, John Wiley & Sons (1995), Chapter 16. For fragments of placental bikunin containing a single Kunitz inhibitor domain such as bikunin (7-64) and (102-159), yeast and E. coli expression systems are preferable, with yeast systems being most preferred. Typically, yeast expression would be carried out as described in U.S. Pat. No. 5,164,482 for aprotinin variants and adapted in Example 5 of the present specification for placental bikunin (102-159). E. coli expression could be carried out using the methods described in U.S. Pat. No. 5,032,573. Use of mammalian and yeast systems are most preferred for the expression of larger placental bikunin variants containing both inhibitor domains such as the variant bikunin (7-159).

DNA encoding variants of placental bikunin that possess amino acid substitution of the natural amino sequence can be prepared for expression of recombinant protein using the methods of Kunkel T. A., (1985) Proc. Natl. Acad. Sci USA 82, pp 488-492. Briefly, the DNA to be mutagenized is cloned into a single stranded bacteriophage vector such as M13. An oligonucleotide spanning the region to be changed and encoding the substitution is hybridized to the single stranded DNA and made double stranded by standard molecular biology techniques. This DNA is then transformed into an appropriate bacterial host and verified by dideoxynucleotide sequencing. The correct DNA is then cloned into the expression plasmid. Alternatively, the target DNA may be mutagenized by standard PCR techniques, sequenced, and inserted into the appropriate expression plasmid.

The following particular examples are offered by way of illustration, and not limitation, of certain aspects and preferred embodiments of the instant invention. All patents, patent applications and literature references cited in this application are incorporated by reference in their entirety.

### EXAMPLE 1

Preparation of Synthetic Placental Bikunin (102-159)

Materials and methods/Reagents used. The fluorogenic substrate Tos-Gly-Pro-Lys-AMC was purchased from Bachem BioScience Inc (King of Prussia, Pa.). PNGB, Pro-Phe-Arg-AMC, Ala-Ala-Pro-Met-AMC, bovine trypsin (type III), human plasma kallikrein, and human plasmin were from Sigma (St. Louis, Mo.).

Recombinant aprotinin (Trasylol®) was from Bayer AG (Wuppertal, Germany). Pre-loaded Gln Wang resin was from Novabiochem (La Jolla, Calif.). Thioanisole, ethanedithiol and t-butyl methyl ether was from Aldrich (Milwaukee, Wis.).

Quantification of Functional Placental Bikunin (7-64) (SEQ ID NO: 4) and (102-159)

The amount of trypsin inhibitory activity present in the refolded sample at various stages of purification was measured using GPK-AMC as a substrate. Bovine trypsin (200 pmoles) was incubated for 5 min at 37% C with bikunin (7-64) or (102-159), from various stages of purification, in buffer A (50 mM Hepes, pH 7.5, 0.1 M NaCl, 2 mM CaCl₂ and 0.01% triton X-100). GPK-AMC was added (20 μM final) and the amount of coumarin produced was determined by measuring the fluorescence (ex=370 nm, em=432 nm) on a Perkin-Elmer LS-50B fluorimeter over a 2 min. period. For samples being tested the % inhibition for each was calculated according to equation 1; where R₀ is the rate of fluorescence increase in the presence of inhibitor and R₁ is the rate determined in the absence of added sample. One unit of activity for the inhibitor is defined as the amount needed to achieve 50% inhibition in the assay using the conditions as described.
% inhibition=100×[1−R₀/R₁] (1)

Synthesis. Placental bikunin (102-159 ID NO: 6) was synthesized on an Applied Biosystems model 420A peptide synthesizer using NMP-HBTU Fmoc chemistry. The peptide was synthesized on pre loaded Gln resin with an 8-fold excess of amino acid for each coupling. Cleavage and deprotection was performed in 84.6% trifluoroacetic acid (TFA), 4.4% thioanisole, 2.2% ethanedithiol, 4.4% liquified phenol, and 4.4% H2O for 2 hours at room temperature. The crude peptide was precipitated, centrifuged and washed twice in t-butyl methyl ether. The peptide was purified on a Dynamax 60A C18 reverse-phase HPLC column using a TFA/acetonitrile gradient. The final preparation (61.0 mg) yielded the correct amino acid composition and molecular mass by Electrospray mass spectroscopy (MH+=6836.1; calcd=6835.5) for the predicted sequence:

YEEYCTANAV TGPCRASFPR WYFDVERNSC NNFIYGGCRG NKNSYRSEEA CMLRCFRQ (SEQ ID NO.: 6)

Purification. Refolding of placental bikunin (102-159) was performed according to the method of Tam et al., (J. Am. Chem. Soc. 1991, 113; 6657-62). A portion of the purified peptide (15.2 mg) was dissolved in 4.0 ml of 0.1 M Tris, pH 6.0, and 8 M urea. Oxidation of the disulfides was accomplished by dropwise addition of a solution containing 23% DMSO, and 0.1 M Tris, pH 6.0 to obtain a final concentration of 0.5 mg/ml peptide in 20% DMSO, 0.1 M Tris, pH 6.0, and 1 M urea. The solution was allowed to stir for 24 hr at 25° C. after which it was diluted 1:10 in buffer containing 50 mM Tris, pH 8.0, and 0.1 M NaCl. The material was purified using a kallikrein affinity column made by covalently attaching 30 mg of bovine pancreatic kallikrein (Bayer AG) to 3.5 mls of CNBr activated Sepharose (Pharmacia) according to the manufacturers instructions. The refolded material was loaded onto the affinity column at a flow rate of 1 ml/min and washed with 50 mM Tris, pH 8.0, and 0.1 M NaCl until absorbance at 280 nm of the wash could no longer be detected. The column was eluted with 3 volumes each of 0.2 M acetic acid, pH 4.0 and 1.7. Active fractions were pooled (see below) and the pH of the solution adjusted to 2.5. The material was directly applied to a Vydac C18 reverse-phase column (5 micron, 0.46×25 cm) which had been equilibrated in 22.5% acetonitrile in 0.1% TFA. Separation was achieved using a linear gradient of 22.5 to 40% acetonitrile in 0.1% TFA at 1.0 ml/min over 40 min. Active fractions were pooled, lyophilized, redissolved in 0.1% TFA, and stored at −20° C. until needed.

Results. Synthetic placental bikunin (102-159) was refolded using 20% DMSO as the oxidizing agent as described above, and purified by a 2-step purification protocol as shown below, to yield an active trypsin inhibitor (Table 1 below).

TABLE 1
Table 1 Purification table for the isolation of synthetic
placental bikunin (102-159)

| Purification | | | | | SpA | |
|---|---|---|---|---|---|---|
| Step | Vol (ml) | mg/ml | mg | Units^{c} (U) | (U/mg) | Yield |
| 8.0 M Urea | 4.0 | 3.75^{a} | 15.0 | 0 | 0 | — |
| 20% DMSO | 32.0 | 0.47^{a} | 15.0 | 16,162 | 1,078 | 100 |
| Kallikrein | 9.8 | 0.009^{b} | 0.09 | 15,700 | 170,000 | 97 |
| affinity | | | | | | |
| C18 | 3.0 | 0.013^{ab} | 0.04 | 11,964 | 300,000 | 74 |

^{a}Protein determined by AAA.
^{b}Protein determined by OD280 nm using the extinction coefficient determined for the purified protein (1.7 × 10⁴ Lmol⁻¹ cm⁻¹).
^{c}One Unit is defined as the amount of material required to inhibit 50% of trypsin activity in a standard assay.

Chromatography of the crude refolded material over an immobilized bovine pancreatic kallikrein column selectively isolated 6.0% of the protein and 97% of the trypsin inhibitory activity present. Subsequent chromatography using C18 reverse-phase yielded a further purification of 2-fold, with an overall recovery of 74%. On RPHPLC, the reduced and refolded placental bikunin (102-159), exhibited elution times of 26.3 and 20.1 minutes, respectively. Mass spectroscopy analysis of the purified material revealed a molecular mass of 6829.8; a loss of 6 mass units from the starting material. This demonstrates the complete formation of the 3 disulfides predicted from the peptide sequence.

The isoelectric points of the purified, refolded synthetic placental bikunin (102-159) was determined using a Multiphor II Electrophoresis System (Pharmacia) run according to the manufacturers suggestions, together with pI standards, using a precast Ampholine® PAGplate (pH 3.5 to 9.5) and focused for 1.5 hrs. After staining, the migration distance from the cathodic edge of the gel to the different protein bands was measured. The pI of each unknown was determined by using a standard curve generated by a plot of the migration distance of standards versus the corresponding pI's. With this technique, the pI of placental bikunin (102-159) was determined to be 8.3, in agreement with the value predicted from the amino acid sequence. This is lower than the value of 10.5 established for the pi of aprotinin. (Tenstad et al., 1994, Acta Physiol. Scand. 152: 33-50).

### EXAMPLE 2

Preparation of Synthetic Placental Bikunin (7-64)

Placental bikunin (7-64) (SEQ ID NO: 4) was synthesized, refolded and purified essentially as described for placental bikunin (102-159) (SEQ ID NO: 6) in Example 1 but with the following modifications: during refolding, the synthetic peptide was stirred for 30 hr as a solution in 20% DMSO at 25° C.; purification by C18 RP-HPLC was achieved with a linear gradient of 25 to 45% acetonitrile in 0.1% TFA over 40 min (1 ml/min). Active fractions from the first C18 run were reapplied to the column and fractionated with a linear gradient (60 min, 1 ml/min) of 20 to 40% acetonitrile in 0.1% TFA.

Results. The final purified reduced peptide exhibited an MH+=6563, consistent with the sequence:

(SEQ ID NO.: 4)

IHDFCLVSKV VGRCRASMPR WWYNVTDGSC QLFVYGGCDG
NSNNYLTKEE
CLKKCATV

The refolding and purification yielded a functional Kunitz domain that was active as an inhibitor of trypsin (Table 2 below).

TABLE 2A
Table 2A Purification table for the isolation of synthetic
placental bikunin (7-64)

| Purification | | | | | SpA | |
|---|---|---|---|---|---|---|
| Step | Vol (ml) | mg/ml | mg | Units^{c} (U) | (U/mg) | Yield |
| 8.0 M Urea | 8.0 | 2.5 | 20.0 | 0 | 0 | — |
| 20% DMSO | 64.0 | 0.31 | 20.0 | 68,699 | 3,435 | 100 |
| Kall affinity | 11.7 | 0.10 | 1.16 | 43,333 | 36,110 | 62 |
| pH 4.0 | | | | | | |
| Kall affinity | 9.0 | 0.64 | 5.8 | 4972 | 857 | 7.2 |
| pH 1.7 | | | | | | |
| C18-1 | 4.6 | 0.14 | 0.06 | 21,905 | 350,143 | 31.9 |
| C18-2 | 1.0 | 0.08 | 0.02 | 7,937 | 466,882 | 11.5 |

The purified refolded protein exhibited an MH+=6558, i.e. 5±1 mass units less than for the reduced peptide. This demonstrates that refolding caused the formation of at least one appropriate disulfide bond.

The pI of placental bikunin (7-64) was determined using the methods employed to determine the pI of placental bikunin (102-159). Placental bikunin (7-64) exhibited a pI that was much higher than the predicted value (pI=7.9). Refolded placental bikunin (7-64) migrated to the cathodic edge of the gel (pH 9.5) and an accurate pI could not be determined under these conditions.

Continued Preparation of Synthetic Placental Bikunin (7-64)

Because the synthetic placental bikunin (7-64) may not have undergone complete deprotection prior to purification and refolding, refolding was repeated using protein which was certain to be completely deprotected. Placental bikunin (7-64) was synthesized, refolded and purified essentially as described for placental bikunin (102-159) but with the following modifications: during refolding, the synthetic peptide (0.27 mg/ml) was stirred for 30 hr as a solution in 20% DMSO at 25 C; purification by C18 RP-HPLC was achieved with a linear gradient of 22.5 to 50% acetonitrile in 0.1% TFA over 40 min (1 ml/min).

Results. The final purified reduced peptide exhibited an MH+=6567.5, consistent with the sequence:

(SEQ ID NO.: 4)

IHDFCLVSKV VGRCRASMPRW WYNVTDGSC QLFVYGGCDG
NSNNYLTKEE
CLKKCATV

The refolding and purification yielded a functional Kunitz domain that was as active as an inhibitor of trypsin (Table 2B below).

TABLE 2B
Table 2B Purification table for the isolation of synthetic
placental bikunin (7-64)

| Purification | | | | | SpA | |
|---|---|---|---|---|---|---|
| Step | Vol (ml) | mg/ml | mg | Units^{c} (U) | (U/mg) | Yield |
| 8.0 M Urea | 4.9 | 2.1 | 10.5 | 0 | 0 | — |
| 20% DMSO | 39.0 | 0.27 | 10.5 | 236,000 | 22,500 | 100 |
| Kallikrein | 14.5 | 0.3 | 0.43 | 120,000 | 279,070 | 50.9 |
| Affinity (pH 2) | | | | | | |
| C18 Reverse | 0.2 | 1.2 | 0.24 | 70,676 | 294,483 | 30.0 |
| Phase | | | | | | |

The purified refolded protein exhibited an MH+=6561.2, i.e. 6.3 mass units less than for the reduced peptide. This demonstrates that refolding caused the formation of the expected three disulfide bonds.

The pI of refolded placental bikunin (7-64) was determined using the methods employed to determine the pI of placental bikunin (102-159). Refolded placental bikunin (7-64) exhibited a pI of 8.85, slightly higher than the predicted value (pI=7.9).

### EXAMPLE 3

In Vitro Specificity of Functional Placental Bikunin Fragment (102-159)

Proteases. Bovine trypsin, human plasmin, and bovine pancreatic kallikrein quantitation was carried out by active site titration using p-nitrophenyl p′-guanidinobenzoate HCl as previously described (Chase, T., and Shaw, E., (1970) Methods Enzmol. 19, 20-27). Human kallikrein was quantitated by active site titration using bovine aprotinin as a standard and PFR-AMC as a substrate assuming a 1:1 complex formation. The Kₘ for GPK-AMC with trypsin and plasmin under the conditions used for each enzyme was 29 μM and 726 μM, respectively; the Kₘ for PFR-AMC with human plasma kallikrein and bovine pancreatic kallikrein was 457 μM and 81.5 M, respectively; the Kₘ for AAPR-AMC with elastase was 1600 μM. Human tissue kallikrein (Bayer, Germany) quantification was carried out by active site titration using p′nitrophenyl p′-guanidinobenzoate HCl as previously described (Chase, T., and Shaw, E., (1970) Methods Enzmol. 19, 20-27).

Inhibition Kinetics: The inhibition of trypsin by placental bikunin (102-159) (described in Example 1) or aprotinin was measured by the incubation of 50 pM trypsin with placental bikunin (102-159) (0-2 nM) or aprotinin (0-3 nM) in buffer A in a total volume of 1.0 ml. After 5 min. at 37° C., 15 μl of 2 mM GPK-AMC was added and the change in fluorescence (as above) was monitored. The inhibition of human plasmin by placental bikunin (102-159) and aprotinin was determined with plasmin (50 pM) and placental bikunin (102-159) (0-10 nM) or aprotinin (0-4 nM) in buffer containing 50 mM Tris-HCl (pH 7.5), 0.1 M NaCl, and 0.02% triton x-100. After 5 min. incubation at 37° C., 25 μl of 20 mM GPK-AMC was added and the change in fluorescence monitored. The inhibition of human plasma kallikrein by placental bikunin (102-159) or aprotinin was determined using kallikrein (2.5 nM) and placental bikunin (102-159) (0-3 nM) or aprotinin (0-45 nM) in 50 mM Tris-HCl (pH 8.0), 50 mM NaCl, and 0.02% triton x-100. After 5 min. at 37° C. 15 μl of 20 mM PFR-AMC was added and the change in fluorescence monitored. The inhibition of bovine pancreatic kallikrein by placental bikunin (102-159) and aprotinin was determined in a similar manner with kallikrein (92 pM), placental bikunin (102-159) (0-1.6 nM) and aprotinin (0-14 pM) and a final substrate concentration of 100 μM. The apparent inhibition constant Kᵢ* was determined using the nonlinear regression data analysis program Enzfitter software (Biosoft, Cambridge, UK): The kinetic data from each experiment were analyzed in terms of the equation for a tight binding inhibitor:
Vᵢ/Vₒ=1−(E₀+I₀+Kᵢ*−[(E₀+1₀+Kᵢ*)²−4E₀I₀]^{1/2})/2E₀ (2)
where Vᵢ/V₀ is the fractional enzyme activity (inhibited vs. uninhibited rate), and E0 and I₀ are the total concentrations of enzyme and inhibitor, respectively. Ki values were obtained by correcting for the effect of substrate according to the equation:
Kᵢ=Kᵢ*/(1+[S₀]/Kₘ) (3)
(Boudier, C., and Bieth, J. G., (1989) Biochim Biophys Acta. 995: 36-41)

For the inhibition of human neutrophil elastase by placental bikunin (102-159) and aprotinin, elastase (19 nM) was incubated with placental bikunin (102-159) (150 nM) or aprotinin (0-7.5 M) in buffer containing 0.1 M Tris-HCl (pH 8.0), and 0.05% triton X-100. After 5 min at 37% C, AAPM-AMC (500 μM or 1000 μM) was added and the fluorescence measured over a two-minute period. Ki values were determined from Dixon plots of the form 1/V versus [1] performed at two different substrate concentrations (Dixon et al., 1979).

The inhibition of human tissue kallikrein by aprotinin, placental bikunin fragment (7-64) or placental bikunin fragment (102-159) was measured by the incubation of 0.35 nM human tissue kallikrein with placental bikunin (7-64) (0-40 nM) or placental bikunin (102-159) (0-2.5 nM), or aprotinin (0-0.5 nM) in a 1 ml reaction volume containing 50 mM Tris-HCl buffer pH 9.0, 50 mM NaCl, and 0.1% triton x-100. After 5 min. at 37° C., 5 ul of 2 mM PFR-AMC was added achieving 10 uM final and the change in fluorescence monitored. The Kₘ for PFR-AMC with human tissue kallikrein under the conditions employed was 5.7 uM. The inhibition of human factor Xa (American Diagnostica, Inc, Greenwich, Conn.) by synthetic placental bikunin (102-159), recombinant placental bikunin, and aprotinin was measured by the incubation of 0.87 nM human factor Xa with increasing amounts of inhibitor in buffer containing 20 mM Tris (pH 7.5), 0.1 M NaCl, and 0.1% BSA. After 5 min. at 37° C., 30 ul of 20 mM LGR-AMC (Sigma) was added and the change in fluorescence monitored. The inhibition of human urokinase (Sigma) by Kunitz inhibitors was measured by the incubation of urokinase (2.7 ng) with inhibitor in a total volume of 1 ml buffer containing 50 mM Tris-HCl (pH 8.0), 50 mM NaCl, and 0.1% Triton x-100. After 5 min. at 37° C., 35 ul of 20 mM GGR-AMC (Sigma) was added and the change in fluorescence monitored. The inhibition of Factor XIa (from Enzyme Research Labs, Southbend, Ind.) was measured by incubating FXIa (0.1 nM) with either 0 to 800 nM placental bikunin (7-64), 0 to 140 nM placental bikunin (102-159) or 0 to 40 uM aprotinin in buffer containing 50 mM Hepes pH 7.5, 100 mM NaCl, 2 mM CaCl2, 0.01% triton x-100, and 1% BSA in a total volume of 1 ml. After 5 min at 37 C, 10 ul of 40 mM Boc-Glu(OBzl)-Ala-Arg-AMC (Bachem Biosciences, King of Prussia, Pa.) was added and the change in fluorescence monitored.

Results: A direct comparison of the inhibition profiles of placental bikunin (102-159) and aprotinin was made by measuring their inhibition constants with various proteases under identical conditions. The Kᵢ values are listed in Table 3 below.

TABLE 3
Table 3 Ki values for the inhibition of various proteases
by bikunin (102-159)

| | | | | |
|---|---|---|---|---|
| | Bikunin | | | |
| Protease | (102-159) | Aprotinin | Substrate | Km |
| (concentration) | Ki (nM) | Ki (nM) | (concentration) | (mM) |

| | | | | |
|---|---|---|---|---|
| Trypsin (48.5 pM) | 0.4 | 0.8 | GPK-AMC | 0.022 |
| | | | (0.03 mM) | |
| Chymotrypsin | 0.24 | 0.86 | AAPF-pNA | 0.027 |
| (5 nM) | | | (0.08 mM) | |
| Bovine Pancreatic | 0.4 | 0.02 | PFR-AMC | 0.08 |
| Kallikrein | | | (0.1 mM) | |
| (92.0 pM) | | | | |
| Human Plasma | 0.3 | 19.0 | PFR-AMC | 0.46 |
| Kallikrein (2.5 nM) | | | (0.3 mM) | |
| Human Plasmin (50 | 1.8 | 1.3 | GPK-AMC | 0.73 |
| pM) | | | (0.5 mM) | |
| Human Neutrophil | 323.0 | 8500.0 | AAPM-AMC | 1.6 |
| Elastase (19 nM) | | | (1.0 μM) | |
| Factor XIIa | >300.0 | 12,000.0 | PFR-AMC | 0.35 |
| | | | (0.2 μM) | |
| Human Tissue | 0.13 | 0.004 | PFR-AMC | 0.0057 |
| Kallikrein | | | (10 μM) | |
| (0.35 nM) | | | | |
| factor Xa (0.87 nM) | 274 | N.I. | LGR-AMC | N.D. |
| | | at 3 μM | (0.6 mM) | |
| Urokinase | 11000 | 4500 | GGR-AMC | N.D. |
| | | | (0.7 mM) | |
| factor Xia (0.1 nM) | 15 | 288 | E(OBz)AR- | 0.46 |
| | | | AMC | |
| | | | (0.4 mM) | |

Placental bikunin (102-159) and aprotinin inhibit bovine trypsin and human plasmin to a comparable extent under the conditions employed. Aprotinin inhibited elastase with a Ki of 8.5 M. Placental bikunin (102-159) inhibited elastase with a Ki of 323 nM. The Ki value for the placental bikunin (102-159) inhibition of bovine pancreatic kallikrein was 20-fold higher than that of aprotinin inhibition. In contrast, placental bikunin (102-159) is a more potent inhibitor of human plasma kallikrein than aprotinin and binds with a 56-fold higher affinity.

Because placental bikunin (102-159) is greater than 50 times more potent than Trasylol® as an inhibitor of kallikrein, smaller amounts of human placental bikunin, or fragments thereof (i.e. placental bikunin (102-159)) are needed than Trasylol® in order to maintain the effective patient doses of inhibitor in KIU. This reduces the cost per dose of the drug and reduces the likelihood of adverse nephrotoxic effects upon re-exposure of the medicament to patients. Furthermore, the protein is human derived, and thus much less immunogenic in man than aprotinin which is derived from cows. This results in significant reductions in the risk of incurring adverse immunologic events upon re-exposure of the medicament to patients.

### EXAMPLE 4

In Vitro Specificity of Functional Placental Bikunin Fragment (7-64)

In vitro specificity of the functional human placental bikunin (7-64) described in Example 2 was determined using the materials and methods as described in the Examples above.

Results: The table below shows the efficacy of placental bikunin (7-64) as an inhibitor of various serine proteases in vitro. Data is shown compared against data obtained for screening inhibition using either placental bikunin (102-159), or aprotinin (Trasylol®).

TABLE 4A
Table 4A Ki values for the inhibition of various proteases
by bikunin(7-64)

| Protease | bikunin(7-64) | Aprotinin | Bikunin (102-159) |
|---|---|---|---|
| (concentration) | Ki (nM) | Ki (nM) | Ki (nM) |
| Trypsin (48.5 pM) | 0.17 | 0.8 | 0.4 |
| Bovine Pancreatic | 0.4 | 0.02 | 0.4 |
| Kallikrein (92.0 pM) | | | |
| Human Plasma | 2.4 | 19.0 | 0.3 |
| Kallikrein (2.5 nM) | | | |
| Human Plasmin | 3.1 | 1.3 | 1.8 |
| (50 pM) | | | |
| Bovine chymotrypsin | 0.6 | 0.9 | 0.2 |
| (5 nM) | | | |
| Factor XIIa | >300 | 12000 | >300 |
| elastase | >100 | 8500 | 323 |

The results show that the amino acid sequence encoding placental bikunin (7-64) can be refolded to obtain an active serine protease inhibitor that is effective against at least four trypsin-like serine proteases.

Table 4B below also shows the efficacy of refolded placental bikunin (7-64) as an inhibitor of various serine proteases in vitro. Refolded placental bikunin (7-64) was prepared from protein that was certain to be completely deprotected prior to purification and refolding. Data is shown compared against data obtained for screening inhibition using either placental bikunin (102-159), or aprotinin (Trasylol®).

TABLE 4B
Table 4B Ki values for the inhibition of various proteases
by refolded bikunin (7-64)

| Protease | bikunin (7-64) | Aprotinin | bikunin (102-159) |
|---|---|---|---|
| (concentration) | Ki (nM) | Ki (nM) | Ki (nM) |
| Trypsin (50 pM) | 0.2 | 0.8 | 0.3 |
| Human Plasma | 0.7 | 19.0 | 0.7 |
| Kallikrein (0.2 nM) | | | |
| Human Plasmin | 3.7 | 1.3 | 1.8 |
| (50 pM) | | | |
| Factor XIIa | not done | 12,000 | 4,500 |
| Factor XIa (0.1 | 200 | 288 | 15 |
| nM) | | | |
| Human Tissue | | | |
| Kallikrein | 2.3 | 0.004 | 0.13 |

Surprisingly, placental bikunin (7-64) was more potent than aprotinin at inhibiting human plasma kallikrein, and at least similar in efficacy as a plasmin inhibitor. These data show that placental bikunin (7-64) is at least as effective as aprotinin, using in vitro assays, and that one would expect better or similar potency in vivo.

### EXAMPLE 5

Expression of Placental Bikunin Variant (102-159) in Yeast

The DNA sequence encoding placental bikunin 102-159 (SEQ ID NO.: 6) was generated using synthetic oligonucleotides. The final DNA product consisted (5′ to 3′) of 15 nucleotides from the yeast a-mating factor propeptide sequence fused to the in-frame cDNA sequence encoding placental bikunin (102-159), followed by an in-frame stop codon. Upon cloning into a yeast expression vector pS604, the cDNA would direct the expression of a fusion protein comprising an N-terminal yeast a-mating factor propeptide fused to the 58 amino acid sequence of placental bikunin (102-159). Processing of this fusion protein at a KEX-2 cleavage site at the junction between the a-mating factor and Kunitz domain was designed to liberate the Kunitz domain at its native N-terminus.

A 5′ sense oligonucleotide of the following sequence and containing a HindIII site for cloning was synthesized:

(SEQ ID NO.: 42)

GAA GGG GTA AGC TTG GAT AAA AGA TAT GAA GAA TAC
TGC ACC GCC
AAC GCA GTC ACT GGG CCT TGC CGT GCA TCC TTC CCA
CGC TGG TAC
TTT GAC GTG GAG AGG

A 3′ antisense oligonucleotide of the following sequence and containing both a BamHI site for cloning and a stop codon was synthesized:

(SEQ ID NO.: 43)

CGC GGA TCC CTA CTG GCG GAA GCA GCG GAG CAT GCA
GGC CTC CTC
AGA GCG GTA GCT GTT CTT ATT GCC CCG GCA GCC TCC
ATA GAT GAA
GTT ATT GCA GGA GTT CCT CTC CAC GTC AAA GTA CCA
GCG

The oligonucleotides were dissolved in 10 mM Tris buffer pH 8.0 containing 1 mM EDTA, and 12 ug of each oligo were added combined and brought to 0.25M NaCl. To hybridize, the oligonucleotides were denatured by boiling for 5 minutes and allowed to cool from 65° C. to room temp over 2 hrs. Overlaps were extended using the Klenow fragment and digested with HindIII and BamHI. The resulting digested double stranded fragment was cloned into pUC19 and sequence confirmed. A clone containing the fragment of the correct sequence was digested with BamHI/HindIII to liberate the bikunin containing fragment with the following+strand sequence:

(SEQ ID.: 44)

GAA GGG GTA AGC TTG GAT AAA AGA TAT GAA GAA TAC
TGC ACC GCC
AAC GCA GTC ACT GGG CCT TGC CGT GCA TCC TTC CCA
CGC TGG TAC
TTT GAC GTG GAG AGG AAC TCC TGC AAT AAC TTC ATC
TAT GGA GGC
TGC CGG GGC AAT AAG AAC AGC TAC CGC TCT GAG GAG
GCC TGC ATG
CTC CGC TGC TTC CGC CAG TAG GGA TCC

which was then gel purified and ligated into BamHI/HindIII cut pS604. The ligation mixture was extracted into phenol/chloroform and purified over a S-200 minispin column. The ligation product was directed transformed into yeast strains SC101 and WVL341 and plated on ura selection plates. Twelve colonies from each strain were re-streaked on ura drop out plates. A single colony was inoculated into 2 ml of ura DO media and grown over night at 30° C. Cells were pelleted for 2 minutes at 14000×g and the supernatants evaluated for their content of placental bikunin (102-159).
Detection of Expression of Placental Bikunin (102-159) in Transformed Yeast

Firstly, the supernatants (50 ul per assay) were evaluated for their capacity to inhibit the in vitro activity of trypsin using the assay methods as described in Example 1 (1 ml assay volume). An un-used media only sample as well as a yeast clone expressing an inactive variant of aprotinin served as negative controls. A yeast clone expressing natural aprotinin served as a positive control and is shown for comparison.

The second method to quantify placental bikunin (102-159) expression exploited use of polyclonal antibodies (pAbs) against the synthetic peptide to monitor the accumulation of the recombinant peptide using Western blots. These studies were performed only with recombinants derived from strain SC101, since these produced greater inhibitory activity than recombinants derived from strain WHL341.

To produce the pAb, two 6-8 week old New Zealand White female rabbits (Hazelton Research Labs, Denver, Pa.) were immunized on day zero with 250 ug of purified reduced synthetic placental bikunin (102-159), in Complete Freund's adjuvant, followed by boosts on days 14, 35 and 56 and 77 each with 125 ug of the same antigen in Incomplete Freund's adjuvant. Antiserum used in the present studies was collected after the third boost by established procedures. Polyclonal antibodies were purified from the antiserum over protein A.

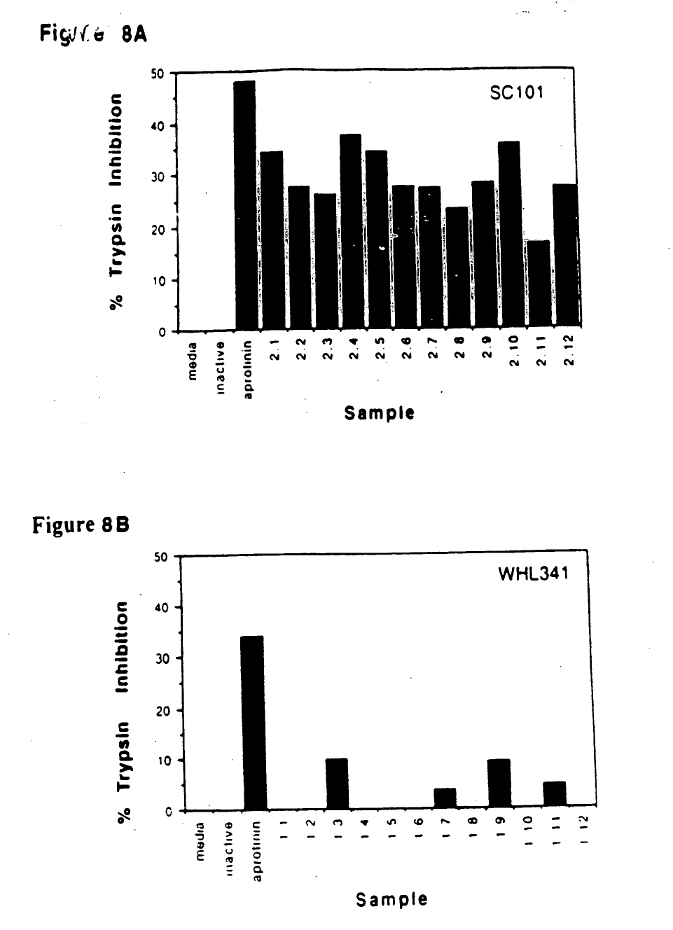

Colonies 2.4 and 2.5 from transformation of yeast SC101 (FIG. 8) as well as an aprotinin control were grown overnight in 50 ml of ura DO media at 30° C. Cells were pelleted and the supernatant concentrated 100-fold using a Centriprep 3 (Amicon, Beverly, Mass.) concentrator. Samples of each (30 μl) were subjected to SDS-PAGE on 10-20% tricine buffered gels (Novex, San Diego, Calif.) using the manufacturers procedures. Duplicate gels were either developed with a silver stain kit (Integrated Separation Systems, Nantick, Mass.) or transferred to nitrocellulose and developed with the purified polyclonal antibody elicited to synthetic bikunin (102-159). Alkaline-phosphatase conjugated goat anti-rabbit antibody was used as the secondary antibody according to the manufacturer's directions (Kirkegaard and Perry, Gaithersburg, Md.).

Purification of Placental Bikunin (102-159) from a Transformed Strain of SC101

Fermentation broth from a 1 L culture of SC101 strain 2.4 was harvested by centrifugation (4,000 g×30 min.) then applied to a 1.0 ml column of anhydrochymotrypsin-sepharose (Takara Biochemical Inc., Calif.), that was previously equilibrated with 50 mM Hepes buffer pH 7.5 containing 0.1M NaCl, 2 mM CaCl2 and 0.01% (v/v) triton X-100. The column was washed with the same buffer but containing 1.0 M NaCl until the A280 nm declined to zero, whereupon the column was eluted with 0.1M formic acid pH 2.5. Eluted fractions were pooled and applied to a C18 column (Vydac, 5 um, 4.6×250 mm) previously equilibrated with 0.1% TFA, and eluted with a 50 min. linear gradient of 20 to 80% acetonitrile in 0.1% TFA. Fractions containing placental bikunin (102-159) were pooled and re-chromatographed on C18 employing elution with a linear 22.5 to 50% acetonitrile gradient in 0.1% TFA.

Results. FIG. 8 shows the percent trypsin activity inhibited by twelve colonies derived from the transformation of each of strains SC101 and WHL341. The results show that all twelve colonies of yeast strain SC101 transformed with the trypsin inhibitor placental bikunin (102-159) had the ability to produce a substantial amount of trypsin inhibitory activity compared to the negative controls both of which showed no ability to inhibit trypsin. The activity is therefore related to the expression of a specific inhibitor in the placental bikunin variant (102-159) transformed cells. The yeast WHL341 samples contained minimal trypsin inhibitory activity. This may be correlated to the slow growth observed with this strain under the conditions employed.

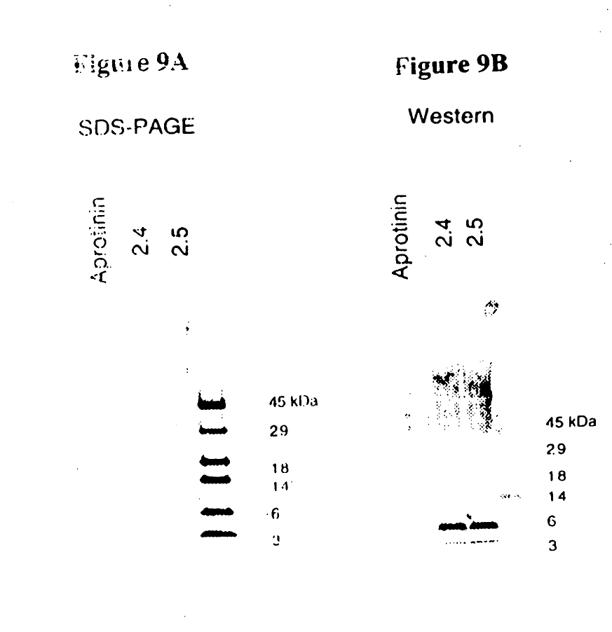

FIG. 9 shows the SDS-PAGE and western analysis of the yeast SC101 supernatants. Silver stained SDS-PAGE of supernatants derived from recombinant yeasts 2.4 and 2.5 expressing placental bikunin (102-159) as well as from the yeast expressing aprotinin yielded a protein band running at approximated 6 kDa, corresponding to the size expected for each recombinant Kunitz inhibitor domain. Western analysis showed that the 6 kDa bands expressed by stains 2.4. and 2.5 reacted with the pAb elicited to placental bikunin (102-159). The same 6 kDa band in the aprotinin control did not react with the same antibody, demonstrating the specificity of the antibody for the placental bikunin variant (102-159).

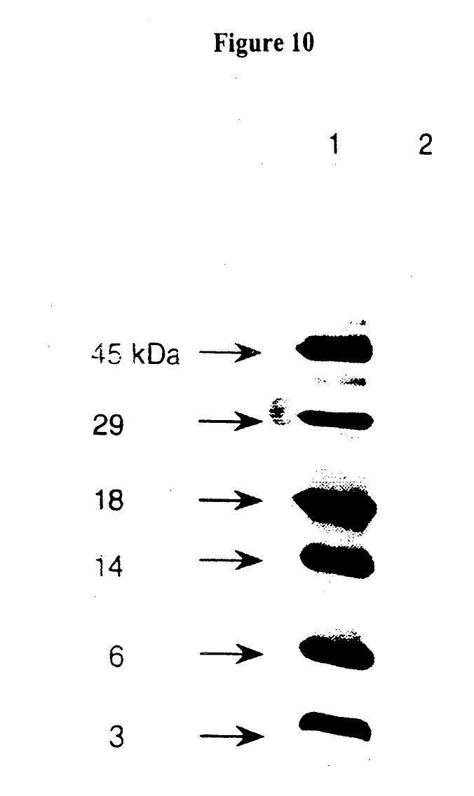

The final preparation of placental bikunin C-terminal domain was highly pure by silver-stained SDS-PAGE (FIG. 10). The overall recovery of broth-derived trypsin inhibitory activity in the final preparation was 31%. N-terminal sequencing of the purified inhibitor indicated that 40% of the protein is correctly processed to yield the correct N-terminus for placental bikunin (102-159) while about 60% of the material contained a portion of the yeast a-mating factor. The purified material comprised an active serine protease inhibitor exhibiting an apparent Ki of 0.35 nM for the in vitro inhibition of plasma kallikrein.

In conclusion, the accumulation both of a protease inhibitor activity and a protein immunochemically related to synthetic bikunin (102-159) in fermentation broth as well as the isolation of placental bikunin (102-159) from one of the transformed lines provided proof of expression of placental bikunin in the recombinant yeast strains described herein, showing for the first time the utility of yeasts for the production of placental bikunin fragments.

Additional constructs were prepared in an effort to augment the expression level of the Kunitz domain contained within placental bikunin 102-159, as well as to increase the yield of protein with the correct N-terminus. We hypothesized that the N-terminal residues of placental bikunin 102-159 (YEEY-) may have presented a cleavage site that is only poorly recognized by the yeast KEX-2 protease that enzymically removes the yeast a-factor pro-region. Therefore, we prepared yeast expression constructs for the production of placental bikunin 103-159 (N-terminus of EEY . . . ), 101-159 (N-terminus of NYEEY . . . ) and 98-159 (DMFNYEEY . . . ) in order to modify the p′ subsites surrounding the KEX-2 cleavage site. To attempt to augment the levels of recombinant protein expression we also used the yeast preferred codons rather than mammalian preferred codons in preparing some of the constructs described below. The constructs were essentially prepared as described above for placental bikunin 102-159 (defined as construct #1) but with the following modifications:

Construct #2 Placental Bikunin 103-159, Yeast Codon Usage

(SEQ ID NO.: 55)

GAAGGGGTAA GCTTGGATAA AAGAGAAGAA TACTGTACTG
CTAATGCTGT
TACTGGTCCA TGTAGAGCTT CTTTTCCAAG ATGGTACTTT
GATGTTGAAA
GA

and 3′ antisense oligonucleotide

(SEQ ID NO.: 56)

ACTGGATCCT CATTGGCGAA AACATCTCAA CATACAGGCT
TCTTCAGATC
TGTAAGAATT TTTATTACCT CTACAACCAC CGTAAATAAA
ATTATTACAA
GAATTTCTTT CAACATCAAA GTACCATCT

were manipulated as described for the production of an expression construct (construct #1 above) for the expression of placental bikunin 102-159
Construct #3 Placental Bikunin 101-159, Yeast Codon Usage

A 5′ sense oligonucleotide

(SEQ ID NO.: 57)

GAAGGGGTAA GCTTGGATAA AAGAAATTAC GAAGAATACT
GTACTGCTAA
TGCTGTTACT GGTCCATGTA GAGCTTCTTT TCCAAGATGG
TACTTTGATG
TTGAAAGA

and the same 3′ antisense oligonucleotide as used for construct #2, were manipulated as described for the production of an expression construct (construct #1 above) for the expression of placental bikunin 102-159.

Construct #4 Placental Bikunin 98-159, Yeast Codon Usage

A 5′ sense oligonucleotide

(SEQ ID NO.: 58)

GAAGGGGTAA GCTTGGATAA AAGAGATATG TTTAATTACG
AAGAATACTG
TACTGCTAAT GCTGTTACTG GTCCATGTAG AGCTTCTTTT
CCAAGATGGT
ACTTTGATGT TGAAAGA

and the same 3′ antisense oligonucleotide as used for construct #2, were manipulated as described for the production of an expression construct (construct #1 above).

Yeast strain SC101 (MATα, ura 3-52, suc 2) was transformed with the plasmids containing each of the above cDNAs, and proteins were expressed using the methods that were described above for the production-of placental bikunin 102-159 with human codon usage. Approximately 250 ml of each yeast culture was harvested, and the supernatant from centrifugation (15 min×3000 RPM) separately subjected to purification over 1 ml columns of kallikrein-sepharose as described above. The relative amount of trypsin inhibitory activity in the applysate, the amount of purified protein recovered and the N-terminal sequence of the purified protein were determined and are listed below in Table 7.

TABLE 7
Table 7 Relative production levels of different proteins containing the
C-terminal Kunitz domain of placental bikunin

| | Relative conc. | N-terminal sequencing | | |
|---|---|---|---|---|
| | of inhibitor in | Amount | | |
| Construct | applysate | (pmol) | sequence | Comments |
| #2 103-159 | none detected | none | none | no expression |
| #3 101-159 | 25% inhibition | none | none | low expression |
| #4 98-159 | 93% inhibition | 910 | DMFNYE- | good expression |
| | | | | correct product |
| #1 102-159 | 82% inhibition | 480 | AKEEGV- | expression of |
| | | | | active incorrectly |
| | | | | processed protein |

The results show that placental bikunin fragments of different lengths that contain the C-terminal Kunitz domain show wide variation in capacity to express functional secreted protein. Constructs expressing fragments 101-159 and 103-159 yielded little or low enzymic activity in the supernatants prior to purification, and N-terminal sequencing of 0.05 ml aliquots of each purified fraction yielded undetectable amounts of inhibitor. On the other hand expression either of placental bikunin 102-159 or 98-159 yielded significant amounts of protease activity prior to purification. N-terminal sequencing however showed that the purified protein recovered from expression of 102-159 was once again largely incorrectly processed, exhibiting an N-terminus consistent with processing of the majority of the pre-protein at a site within the yeast α-mating factor pro-sequence. The purified protein recovered from expression of placental bikunin 98-159 however was processed entirely at the correct site to yield the correct N-terminus. Furthermore, nearly twice as much protein was recovered as compared to the recovery of placental bikunin 102-159. Placental bikunin 98-159 thus represents a preferred fragment length for the production of the C-terminal Kunitz domain of placental bikunin by the α-mating factor pre-pro sequence/KEX-2 processing system of S. cerevisiae,

### EXAMPLE 6

Alternative Procedure for Yeast Expression

The 58 amino acid peptide derived from the R74593 translation product can also be PCR amplified from either the R87894-R74593 PCR product cloned into the TA vector™ (Invitrogen, San Diego, Calif.) after DNA sequencing or from human placental cDNA. The amplified DNA product will consist of 19 nucleotides from the yeast α-mating factor leader sequence mated to the R74593 sequence which codes for the YEEY-CFRQ (58 residues) so as to make the translation product in frame, constructing an α-mating factor/Kunitz domain fusion protein. The protein sequence also contains a kex 2 cleavage which will liberate the Kunitz domain at its native N-terminus.

The 5′ sense oligonucleotide which contains a HindIII site for cloning will contain the following sequence:

(SEQ ID NO.: 30)

GCCAAGCTTG GATAAAAGAT ATGAAGAAT ACTGCACCGC CAACGCA

The 3′ antisense oligonucleotide contains a BamHI site for cloning as well as a stop codon and is of the following sequence:

| | |
|---|---|
| GGGGATCCTC ACTGCTGGCG GAAGCAGCGG | (SEQ ID NO.: 31) |
| AGCAT | |

The full 206 nucleotide cDNA sequence to be cloned into the yeast expression vector is of the following sequence:

(SEQ ID NO.: 32)

CCAAGCTTGG ATAAAAGATA TGAAGAATAC TGCACCGCCA
ACGCAGTCAC
TGGGCCTTGC CGTGCATCCT TCCCACGCTG GTACTTTGAC
GTGGAGAGGA
ACTCCTGCAA TAACTTCATC TATGGAGGCT GCCGGGGCAA
TAAGAACAGC
TACCGCTCTG AGGAGGCCTG CATGCTCCGC TGCTTCCGCC
AGCAGTGAGG ATCCCC

After PCR amplification, this DNA will be digested with HindIII, BamHI and cloned into the yeast expression vector pMT15 (see U.S. Pat. No. 5,164,482, incorporated by reference in the entirety) also digested with HindIII and BamHI. The resulting plasmid vector is used to transform yeast strain SC 106 using the methods described in U.S. Pat. No. 5,164,482. The URA 3+ yeast transformants are isolated and cultivated under inducing conditions. The yield of recombinant Placental bikunin variants is determined according to the amount of trypsin inhibitory activity that accumulated in the culture supernatants over time using the in vitro assay method described above. Fermentation broths are centrifuged at 9000 rpm for 30 minutes. The supernatant is then filtered through a 0.4 then a 0.2 p.m filter, diluted to a conductivity of 7.5 ms, and adjusted to pH 3 with citric acid. The sample is then batch absorbed onto 200 ml of S-sepharose fast flow (Pharmacia) in 50 mM sodium citrate pH 3 and stirred for 60 min. The gel is subsequently washed sequentially with 2 L of each of: 50 mM sodium citrate pH 3.0; 50 mM Tris-HCL pH 9.0; 20 mM HEPES pH 6.0. The washed gel is transferred into a suitable column and eluted with a linear gradient of 0 to 1 M sodium chloride in 20 mM HEPES pH 6.0. Eluted fractions containing in vitro trypsin inhibitory activity are then pooled and further purified either by a) chromatography over a column of immobilized anhydrotrypsin (essentially as described in Example 2); b) by chromatography over a column of immobilized bovine kallikrein; or c) a combination of conventional chromatographic steps including gel filtration and/or anion-exchange chromatography.

### EXAMPLE 7

Isolation and Characterization of Native Human Placental Bikunin from Placenta

Bikunin protein was purified to apparent homogeniety from whole frozen placenta (Analytical Biological Services, Inc, Wilmington, Del.). The placenta (740 gm) was thawed to room temperature and cut into 0.5 to 1.0 cm pieces, placed on ice and washed with 600 ml PBS buffer. The wash was decanted and 240 ml of placenta pieces placed into a Waring blender. After adding 300 ml of buffer consisting of 0.1 M Tris (pH 8.0), and 0.1 M NaCl, the mixture was blended on high speed for 2 min, decanted into 750.0 ml centrifuge tubes, and placed on ice. This procedure was repeated until all material was processed. The combined slurry was centrifuged at 4500×g for 60 minutes at 4° C. The supernatant was filtered through cheese cloth and the placental bikunin purified using a kallikrein affinity column made by covalently attaching 70 mg of bovine pancreatic kallikrein (Bayer AG) to 5.0 mls of CNBr activated Sepharose (Pharmacia) according to manufacturers instruction. The material was loaded onto the affinity column at a flow rate of 2.0 ml min and washed with 0.1 M Tris (pH 8.0), 0.1 M NaCl until absorbance at 280 nm of the wash could no longer be detected. The column was further washed with 0.1 M Tris (pH 8.0), 0.5 M NaCl and then eluted with 3 volumes of 0.2 M acetic acid, pH 4.0. Fractions containing kallikrein and trypsin inhibitory (see below) activity were pooled, frozen, and lyophilized. Placental bikunin was further purified by gel-filtration chromatography using a Superdex 75 10/30 (Pharmacia) column attached to a Beckman System Gold HPLC system. Briefly, the column was equilibrated in 0.1 M Tris, 0.15 M NaCl, and 0.1% Triton X-100 at a flow rate of 0.5 ml/min. The lyophilized sample was reconstituted in 1.0 ml of 0.1 M Tris, pH 8.0 and injected onto the gel-filtration column in 200 μl aliquots. Fractions were collected (0.5 ml) and assayed for trypsin and kallikrein inhibitory activity. Active fractions were pooled, and the pH of the solution adjusted to 2.5 by addition of TFA. The material was directly applied to a Vydac C18 reverse-phase column (5 micron, 0.46×25 cm) which had been equilibrated in 20% acetonitrile in 0.1% TFA. Separation was achieved using a linear gradient of 20 to 80% acetonitrile in 0.1% TFA at 1.0 ml/min over 50 minutes after an initial 20 minute wash at 20% acetonitrile in 0.1% TFA. Fractions (1 ml) were collected and assayed for trypsin and kallikrein inhibitory activity. Fractions containing inhibitory activity were concentrated using a speed-vac concentrator (Savant) and subjected to N-terminal sequence analysis.

Functional Assays for Placental Bikunin:

Identification of functional placental bikunin was achieved by measuring its ability to inhibit bovine trypsin and human plasma kallikrein. Trypsin inhibitory activity was performed in assay buffer (50 mM Hepes, pH 7.5, 0.1 M NaCl, 2.0 mM CaCl2, 0.1% Triton x-100) at room temperature in a 96-well microliter plate (Perkin Elmer) using Gly-Pro-Lys-Aminomethylcoumarin as a substrate. The amount of coumarin produced by trypsin was determined by measuring the fluorescence (ex=370 nm, em=432 nm) on a Perkin-Elmer LS-50B fluorimeter equipped with a plate reader. Trypsin (23 μg in 100 μl buffer) was mixed with 20 μl of the sample to be tested and incubated for 10 minutes at 25° C. The reaction was started by the addition of 50 μl of the substrate GPK-AMC (33 μM final) in assay buffer. The fluorescence intensity was measured and the % inhibition for each fraction was determined by:
% inhibition=100×[1−Fo/F1]
where Fo is the fluorescence of the unknown and F1 is the fluorescence of the trypsin only control. Kallikrein inhibitory activity of the fractions was similarly measured using 7.0 nM kallikrein in assay buffer (50 mM Tris, pH 8.0, 50 mM NaCl, 0.1% triton x-100) and 66.0 M Pro-Phe-Arg-AMC as a substrate.
Determination of the In Vitro Specificity of Placental Bikunin

The In vitro specificity of native human placental bikunin was determined using the materials and methods as described in the preceding examples above. Placental bikunin was quantified by active site titration against a known concentration of trypsin using GPK-AMC as a substrate to monitor the fraction of unbound trypsin.

Protein Sequencing

The 1 ml fraction (C18-29 Delaria) was reduced to 300 ml in volume, on a Speed Vac, to reduce the amount of organic solvent. The sample was then loaded onto a Hewlett-Packard miniature biphasic reaction column, and washed with 1 ml of 2% trifluoroacetic acid. The sample was sequenced on a Hewlett-Packard Model G1005A protein sequencing system using Edman degradation. Version 3.0 sequencing methods and all reagents were supplied by Hewlett-Packard. Sequence was confirmed for 50 cycles.

Results. Placental Bikunin was purified to apparent homogeniety by sequential kallikrein affinity, gel-filtration, and reverse-phase chromatography (see purification table below):

TABLE 5
Table 5 Purification table for native Placental Bikunin (1-1791

| | | OD 280 | | Units^{a} | Units/ |
|---|---|---|---|---|---|
| Step | Vol (ml) | (/ml) | OD 280 | (U) | OD 280 |
| Placenta | 1800.0 | 41.7 | 75,060 | 3,000,000 | 40.0 |
| Supernatant | | | | | |
| Kallikrein | 20.0 | 0.17 | 3.36 | 16,000 | 4,880 |
| Affinity pH 4.0 | | | | | |
| Kallikrein | 10.2 | 0.45 | 4.56 | 12,000 | 2,630 |
| Affinity pH 1.7 | | | | | |
| Superdex 75 | 15.0 | 0.0085 | 0.13 | 3,191 | 24,546 |

^{a}One Unit is defined as that amount which inhibits 50% of trypsin activity in a standard assay.

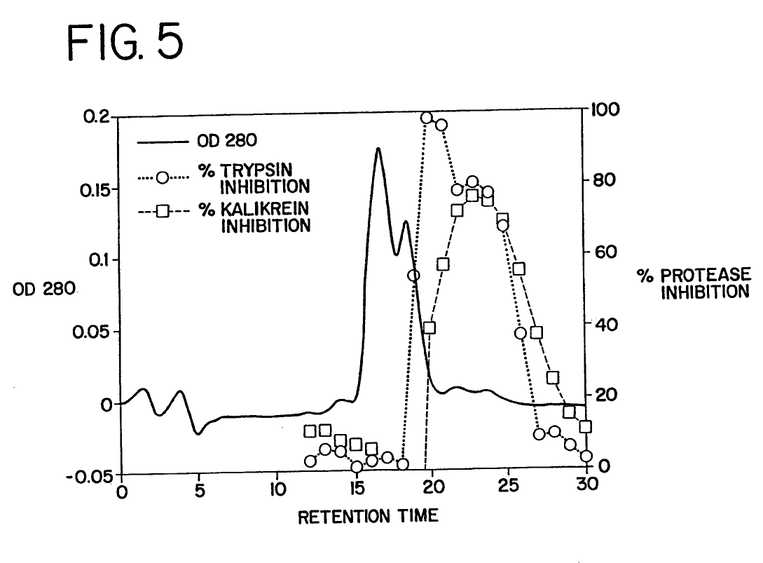

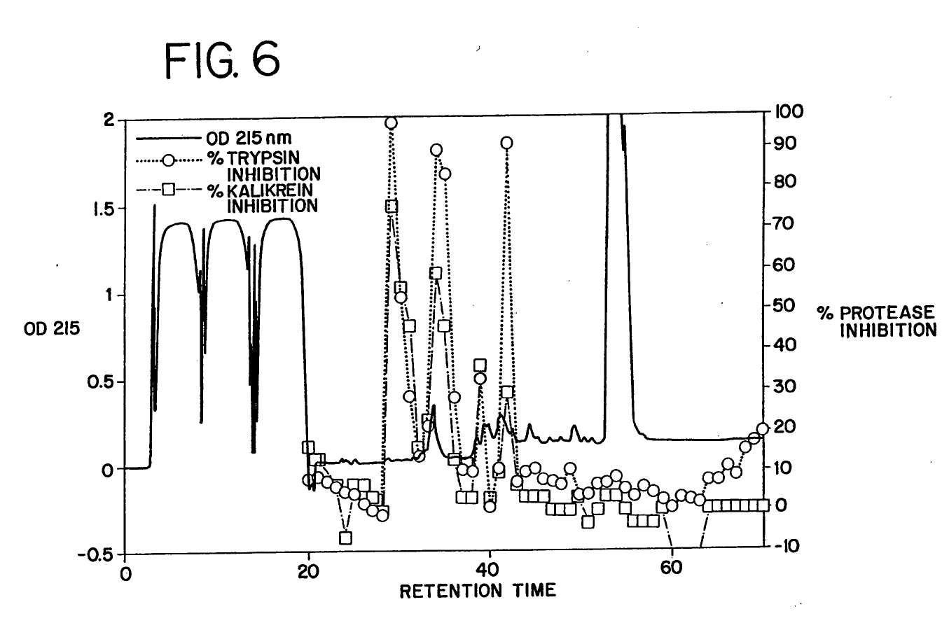

The majority of the kallikrein and trypsin inhibitory activity eluted from the kallikrein affinity column in the pH 4.0 elution. Subsequent gel-filtration chromatography (FIG. 5) yielded a peak of kallikrein and trypsin inhibitory activity with a molecular weight range of 10 to 40 kDa as judged by a standard curve generated by running molecular weight standards under identical conditions. Reverse-phase C18 chromatography (FIG. 6) yielded 4 peaks of inhibitory activity with the most potent eluting at approximately 30% acetonitrile. The activity associated with the first peak to elute from C18 (fraction 29) exhibited an amino acid sequence starting with amino acid 1 of the predicted amino acid sequence of placental bikunin (ADRER . . . ; SEQ ID NO.: 1), and was identical to the predicted sequence for 50 cycles of sequencing (underlined amino acids in FIG. 3). Cysteine residues within this sequence stretch were silent as expected for sequencing of oxidized protein. The cysteine residues at amino acid positions 11 and 20 of mature placental bikunin were later identified from sequencing of the S-pyridylethylated protein whereupon PTH-pyridylethyl-cysteine was recovered at cycles 11 and 20.

Interestingly, the asparagine at amino acid residue number 30 of the sequence (FIG. 3) was silent showing that this site is likely to be glycosylated. Fraction 29 yielded one major sequence corresponding to that of placental bikunin starting at residue #1 (27 pmol at cycle 1) plus a minor sequence (2 pmol) also derived from placental bikunin starting at residue 6 (SIHD . . . ). This shows that the final preparation sequenced in fraction 29 is highly pure, and most likely responsible for the protease inhibitory activity associated with this fraction (FIG. 6).

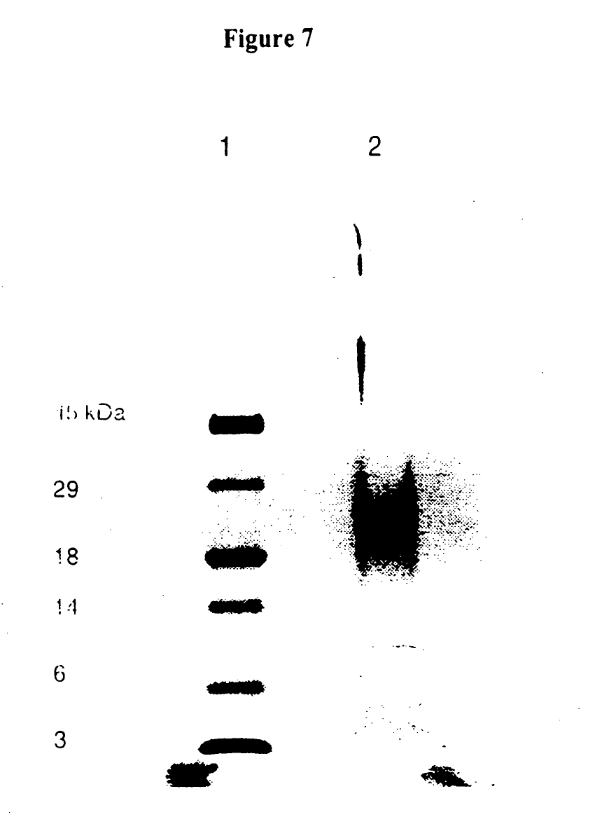

Accordingly, the final preparation of placental bikunin from C18 chromatography was highly pure based on a silver-stained SDS-PAGE analysis (FIG. 7), where the protein migrated with an apparent Mr of 24 kDa on a 10 to 20% acrylamide tricine gel (Novex, San Diego, Calif.) calibrated with the following molecular weight markers: insulin (2.9 kDa); bovine trypsin inhibitor (5.8 kDa); lysozyme (14.7 kDa); β-lactaglobulin (18.4 kDa); carbonic anhydrase (29 kDa); and ovalbumin (43 kDa). The above size of placental bikunin on SDS-PAGE is consistent with that predicted from the full length coding sequence (FIG. 4F).

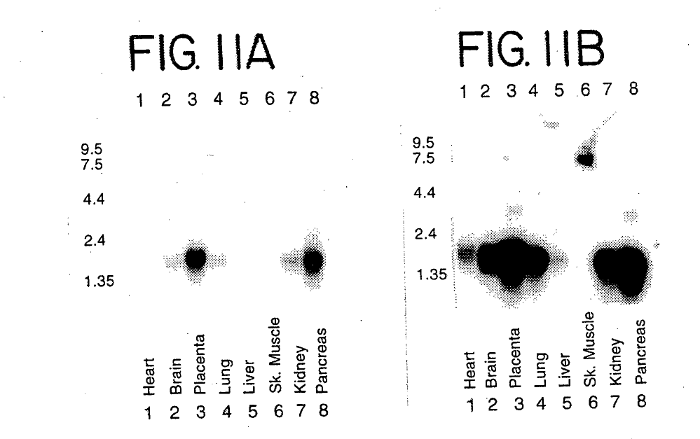

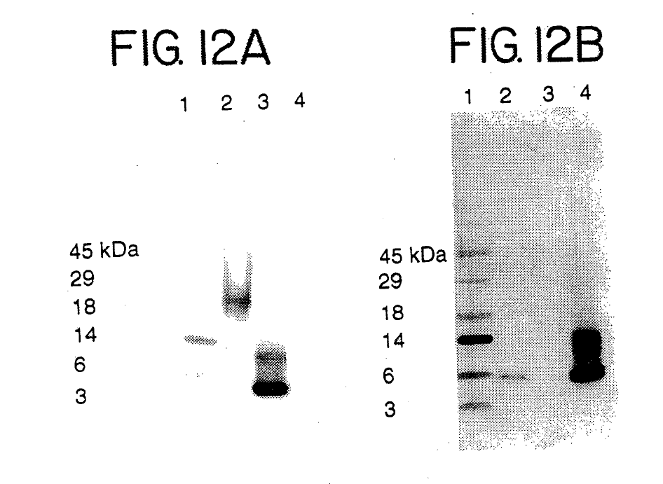

As expected based on the N-terminal sequencing results described above, the purified protein reacted with an antibody elicited to placental bikunin (7-64) to yield a band with the same Mr (FIG. 12A) as observed for the purified preparation detected on gels by silver stain (FIG. 7). However, when the same preparation was reacted with an antibody elicited to synthetic placental bikunin (102-159), a band corresponding to the full length protein was not observed. Rather, a fragment that co-migrated with synthetic bikunin (102-159) of approximately 6 kDa was observed. The simplest interpretation of these results is that the purified preparation had undergone degradation subsequent to purification to yield an N-terminal fragment comprising the N-terminal domain and a C-terminal fragment comprising the C-terminal domain. Assuming that the fragment reactive against antiserum to placental bikunin (7-64) is devoid of the C-terminal end of the full length protein, the size (24 kDa) would suggest a high state of glycosylation.

Table 6 below shows the potency of in vitro inhibition of various serine proteases by placental bikunin. Data are compared with that obtained with aprotinin (Trasylol®).

TABLE 6
Table 6 Ki values for the inhibition of various proteases
by placental bikunin

| Protease | Placental Bikunin | Aprotinin |
|---|---|---|
| (concentration) | Ki (nM) | Ki (nM) |
| Trypsin (48.5 pM) | 0.13 | 0.8 |
| Human Plasmin | 1.9 | 1.3 |
| (50 pM) | | |

The results show that placental bikunin isolated from a natural source (human placenta) is a potent inhibitor of trypsin-like serine proteases.

### EXAMPLE 8

Expression Pattern of Placental Bikunin Amongst Different Human Organs and Tissues

A multiple tissue northern was purchased from Clontech which contained 2 μg of poly A+ RNA from human heart, brain, placenta, lung, liver, skeletal muscle, kidney, and pancreas. Two different cDNA probes were used: 1) a gel purified cDNA encoding placental bikunin (102-159); 2) the 780 base pair PCR-derived cDNA (FIG. 4E) liberated from a TA clone by digestion with EcoRI and gel purified. Each probe was labeled using ³²P-dCTP and a random priming labeling kit from Boehringer Mannheim Biochemicals (Indiana), then used to hybridize to the multiple tissue northern according to the manufacturers specifications. Autoradiographs were generated using Biomax film with an 18 hr exposure time, and developed using a Umax Scanner and scanned using Adobe Photoshop.

Results. The pattern of tissue expression observed using a placental bikunin (102-159) probe (FIG. 11A) or a larger probe containing both Kunitz domains of placental bikunin (FIG. 11B) was essentially the same as might be expected. The placental bikunin mRNA was most abundant in pancreas and placenta. Significant levels were also observed in lung, brain and kidney, while lower levels were observed in heart and liver, and the mRNA was undetectable in skeletal muscle. The transcript size was 1.95 kilobases in all cases, in close agreement with the predicted size of placental bikunin deduced both from EST overlay and cloning of full length cDNA described in preceding sections.

The broad tissue distribution of the mRNA shows that placental bikunin is broadly expressed. Since the protein also contains a leader sequence it would have ample exposure to the human immune system, requiring that it become recognized as a self protein. Additional evidence for a broad tissue distribution of placental bikunin mRNA expression was derived from the fact that some of the EST entries with homology to placental bikunin (FIG. 4B) were derived from human adult and infant brain, and human retina, breast, ovary, olfactory epithelium, and placenta. It is concluded therefore that administration of the native human protein to human patients would be unlikely to elicit an immune response.

Interestingly, the expression pattern of placental bikunin is somewhat reminiscent of that for bovine aprotinin which is found in high levels in bovine lung and pancreas. To further elucidate the expression pattern of placental bikunin, RT-PCR of total RNA from the following human cells was determined: un-stimulated human umbilical vein endothelial cells (HUVECs), HK-2 (line derived from kidney proximal tubule), TF-1 (erythroleukemia line) and phorbolester (PMA)-stimulated human peripheral blood leukocytes. The probes used: CACCTGATCGCGAGACCCC (sense; SEQ ID NO.: 59);

(sense; SEQ ID NO.: 59)

CACCTGATCGCGAGACCCC;

(antisense; SEQ ID NO.: 60)

CTGGCGGAAGCAGCGGAGCATGC,

were designed to amplify a 600 b.p. placental bikunin encoding cDNA fragment. Comparisons were normalized by inclusion of actin primers to amplify an 800 b.p. actin fragment. Whereas the 800 b.p fragment identified on agarose gels with ethidium bromide was of equal intensity in all lanes, the 600 b.p. placental bikunin fragment was absent from the HUVECs but present in significant amounts in each of the other cell lines. We conclude that placental bikunin is not expressed in at least some endothelial cells but is expressed in some leukocyte populations.

### EXAMPLE 9

Purification and Properties of Placental Bikunin (1-170) Highly Purified from a Baculovirus/Sf9 Expression System

A large fragment of Placental bikunin containing both Kunitz domains (Bikunin (1-170) (SEQ ID NO: 52) was expressed in Sf9 cells as follows. Placental bikunin cDNA obtained by PCR (FIG. 4E) and contained within a TA vector (see previous Examples) was liberated by digestion with HindIII and XbaI yielding a fragment flanked by a 5′ XbaI site and 3′ HindIII site. This fragment was gel purified and then cloned into the M13 mp19 vector (New England Biolabs, Beverly, Mass.). In vitro mutagenesis (Kunkel T. A., (1985) Proc. Natl. Acad. Sci. USA, 82: 488-492) was used to generate a Pst1 site 3′ to the Xbal site at the 5′ end, but 5′ to the sequence encoding the ATG start site, natural placental bikunin signal peptide and mature placental bikunin coding sequence. The oligonucleotide used for the mutagenesis had the sequence:

(SEQ ID NO.: 61)

5′ CGC GTC TCG GCT GAC CTG GCC CTG CAG ATG GCG CAC
GTG TGC GGG 3′

A stop codon (TAG) and BglII/XmaI site was similarly engineered at the 3′ end of the cDNA using the oligonucleotide:

(SEQ ID NO.: 62)

5′ CTG CCC CTT GGC TCA AAG TAG GAA GAT CTT CCC CCC
GGG GGG GTG GTT CTG GCG GGG CTG 3′.

The stop codon was in frame with the sequence encoding placental bikunin and caused termination immediately following the Lysine at amino acid residue 170, thus encoding a truncated placental bikunin fragment devoid of the putative transmembrane domain. The product from digestion with PstI and BglII was isolated and cloned into the BacPac8 vector for expression of Placental bikunin fragment (1-170) which contains both Kunitz domains but which is truncated immediately N-terminal to the putative transmembrane segment.

The expression of Bikunin by Sf-9 insect cells was optimal at a multiplicity of infection of 1 to 1 when the medium was harvested at 72 h post infection. After harvesting, the baculovirus cell culture supernatant (2 L) was adjusted to pH 8.0 by the addition of Tris-HCl. Bikunin was purified by chromatography using a 5 ml bovine pancreatic kallikrein affinity column as previously described in Example 7 for the purification of native placental bikunin from placenta. Eluted material was adjusted to pH 2.5 with TFA and subjected to chromatography on a C18 reverse-phase column (1.0×25 cm) equilibrated in 10% acetonitrile in 0.1% TFA at a flow rate of 1 ml/min. The bikunin was eluted with a linear gradient of 10 to 80% acetonitrile in 0.1% TFA over 40 min. Active fractions were pooled, lyophilized, redissolved in 50 mM Hepes (pH 7.5), 0.1 M NaCl, 2 mM CaCl2, and 0.1% triton x-100, and stored at −20° C. until needed. The concentration of recombinant bikunin was determined by amino acid analysis.

Results. Recombinant bikunin was purified from baculovirus cell culture supernatant using a 2-step purification protocol as shown below, to yield an active trypsin inhibitor (Table 8 below).

TABLE 8
Table 8 Purification of recombinant bikunin from
transformed culture supernatant

| | | | | | Specific |
|---|---|---|---|---|---|
| Purification | Vol | | OD 280 | Units | activity |
| Step | (ml) | OD 280/ml | total | (U) | (U/OD) |
| Supernatant | 2300.0 | 9.0 | 20,700 | 6,150,000 | 297 |
| Kallikrein | 23.0 | 0.12 | 2.76 | 40,700 | 14,746 |
| affinity | | | | | |
| C18 | 0.4 | 3.84 | 1.54 | 11,111 | 72,150 |
| reverse-phase | | | | | |

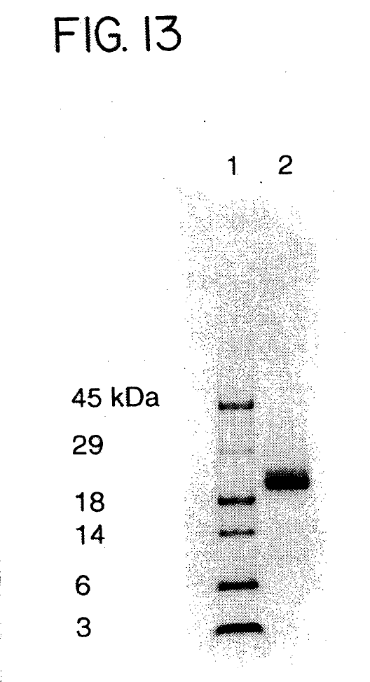

Chromatography of the crude material over an immobilized bovine pancreatic kallikrein affinity column selectively isolated 0.013% of the protein and 0.67% of the trypsin inhibitory activity present. The majority of the trypsin inhibitory activity present in the starting supernatant did not bind to the immobilized kallikrein and is not related to bikunin (results not shown). Subsequent chromatography using C18 reverse-phase yielded a further purification of 5-fold, with a recovery of 0.2%. The final preparation was highly pure by SDS-PAGE (FIG. 13), exhibiting an Mr of 21.3 kDa, and reacted on immunoblots to rabbit anti-placental bikunin 102-159 (not shown). N-terminal sequencing (26 cycles) yielded the expected sequence for mature placental bikunin (FIG. 4F) starting at residue+1 (ADRER . . . ), showing that the signal peptide was correctly processed in Sf9 cells.

Purified placental bikunin from Sf9 cells (100 pmol) was pyridylethyl-alkylated, CNBr digested and then sequenced without resolution of the resulting fragments. Sequencing for 20 cycles yielded the following N-terminii:

| Sequence | Amount | Placental bikunin residue # | |
|---|---|---|---|
| LRCFrQQENPP-PLG | 21 pmol | 154-168 | (SEQ ID NO.: 63) |
| ADRERSIHDFCLVSKVVGRC | 20 pmol | 1-20 | (SEQ ID NO.: 64) |
| FNYeEYCTANAVTGPCRASF | 16 pmol | 100-119 | (SEQ ID NO.: 65) |
| Pr--Y-V-dGS-Q-F-Y-G | 6 pmol | 25-43 | (SEQ ID NO.: 66) |

Thus N-terminii corresponding to each of the expected four fragments were recovered. This confirms that the Sf9 expressed protein contained the entire ectodomain sequence of placental bikunin (1-170).

### EXAMPLE 10

Inhibition Specificity of Purified Placental Bikunin (1-170) Derived from Sf9 Cells.

The in vitro specificity of recombinant bikunin was determined using the materials and methods as described in Examples 3, 4 and 7. In addition, the inhibition of human tissue kallikrein by bikunin was measured by the incubation of 0.35 nM human tissue kallikrein recombinant bikunin in buffer containing 50 mM Tris (pH 9.0), 50 mM NaCl, and 0.01% triton x-100. After 5 min. at 37° C., 5 μl of 2 mM PFR-AMC was added and the change in fluorescence monitored.

Inhibition of tissue plasminogen activator (tPA) was also determined as follows: tPA (single chain form from human melanoma cell culture from Sigma Chemical Co, St Louis, Mo.) was pre-incubated with inhibitor for 2 hr at room temperature in 20 mM Tris buffer pH 7.2 containing 150 mM NaCl, and 0.02% sodium azide. Reactions were subsequently initiated by transfer to a reaction system comprising the following initial component concentrations: tPA (7.5 nM), inhibitor 0 to 6.6 μM, DIle-Lpro-Larg-pNitroaniline (1 mM) in 28 mM Tris buffer pH 8.5 containing 0.004% (v/v) triton x-100 and 0.005% (v/v) sodium azide. Formation of p-Nitroaniline was determined from the A405 nm measured following incubation at 37 C for 2 hr.

The table below show the efficacy of recombinant bikunin as an inhibitor of various serine proteases in vitro. Data is shown compared against data obtained for screening inhibition using either recombinant bikunin, or aprotinin.

TABLE 9
Table 9 Comparisons of Ki values for the inhibition of various proteases
by recombinant placental bikunin (1-170) or aprotinin

| Protease | Recombinant | Aprotinin |
|---|---|---|
| (concentration) | Bikunin Ki (nM) | Ki (nM) |
| Trypsin (48.5 pM) | 0.064 | 0.8 |
| Human Plasma | 0.18 | 19.0 |
| Kallikrein (2.5 nM) | | |
| Human Tissue | 0.04 | 0.004 |
| Kallikrein (0.35 nM) | | |
| Bovine Pancreatic | 0.12 | 0.02 |
| Kallikrein (100 pM) | | |
| Human Plasmin | 0.23 | 1.3 |
| (50 pM) | | |
| factor Xa (0.87 nM) | 180 | 5% Inhibition at 31 M |
| factor XIa (0.1 nM) | 3.0 | 288 |
| tissue plasminogen | <60 | no inhibition at 6.6 μM |
| activator (7.5 nM) | | |
| Tissue Factor VIIa | 800 | no inhibition at 1 μM |

The results show that recombinant bikunin can be expressed in insect cells to yield an active protease inhibitor that is effective against at least five different serine protease inhibitors. Recombinant bikunin was more potent than aprotinin against human plasma kallikrein, trypsin and plasmin. Surprisingly, the recombinant bikunin was more potent that the synthetically derived bikunin fragments (7-64) and (102-159) against all enzymes tested. These data show that recombinant bikunin is more effective than aprotinin, using in vitro assays, and that one would expect better in vivo potency.

Besides measuring the potencies against specific proteases, the capacity of placental bikunin (1-170) to prolong the activated partial thromboplastin time (APTT) was evaluated and compared with the activity associated with aprotinin. Inhibitor was diluted in 20 mM Tris buffer pH 7.2 containing 150 mM NaCl and 0.02% sodium azide and added (0.1 ml) to a cuvette contained within an MLA Electra^{R} 800 Automatic Coagulation Timer coagulometer (Medical Laboratory Automation, Inc., Pleasantville, N.Y.). The instrument was set to APTT mode with a 300 sec. activation time and the duplicate mode. Following addition of 0.1 ml of plasma (Specialty Assayed Reference

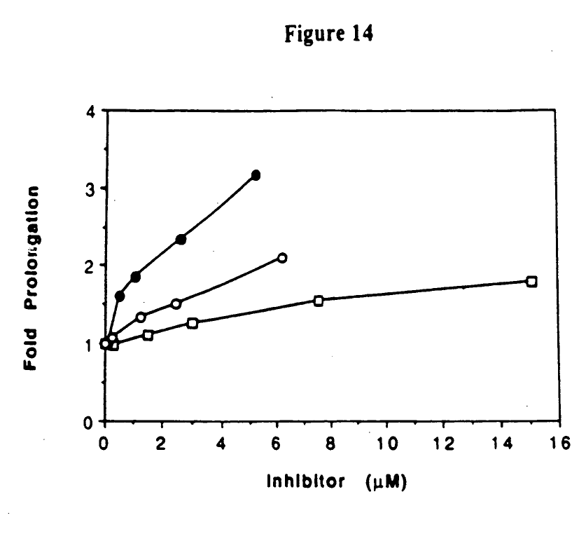

Plasma lot 1-6-5185, Helena Laboratories, Beaumont, Tex.), the APTT reagent (Automated APTT-lot 102345, from Organon Teknika Corp., Durhan, N.C.) and 25 mM CaCl2 were automatically dispensed to initiate clotting, and the clotting time was monitored automatically. The results (FIG. 14) showed that a doubling of the clotting time required approximately 2 μM final aprotinin, but only 0.3 μM Sf9 derived placental bikunin. These data show that placental bikunin is an effective anticoagulant, and useful as a medicament for diseases involving pathologic activation of the intrinsic pathway of coagulation.

### EXAMPLE 11

Measurement of Tracheal Potential Difference in the Guinea-Pig

The aim of this study was to investigate the effect of the Kunitz serine protease inhibitor Bikunin, and the sodium channel blocker amiloride on guinea-pig tracheal potential difference 3 hours post treatment. These agents were delivered into the cephalad trachea by topical instillation. TPD was monitored 2 hours later for 60 minutes. The procedure used in this Example is described in Newton et al. in “Cilia, Mucus and Mucociliary Interactions,” Ed., Baum, G. L. et al., Marcel Dekker, New York, 1998; Newton et al., Ped. Pulm. S17, Abs. 364, 1998).

Materials and Methods/Reagents Used

Aqueous formulations of Bikunin (1-170) (5 and 50 ug/mL (SEQ ID NO: 52)) (as described in Example 17 below) and amiloride (obtained from Sigma Chemicals, St. Louis, Mo., USA)(100 uM) were prepared, sterile filtered and endotoxin tested prior to use. These formulations were prepared in Hank's Balanced salt solution (HBSS) and contained 137 mM NaCl, 3 mM KCl, 3 mM KH₂PO₄, 8 mM Na₂HPO₄, 0.2% Tween-80, pH 7.1) was prepared, sterile filtered and endotoxin tested for use in this example. HBSS was used as a control solution. Hypnorm® (Fentanyl citrate 0.315 mg/mL and Fluanisone 10 mg/mL) was obtained from Janssen Animal Health and Hypnovel® (Midazolam 5 mg/mL) was obtained from Roche. Male Dunkin-Hartley guinea pigs (550-750 g) were supplied by David Hall, UK. Thermistor probes were obtained from Kane-May Ltd, UK.

Induction of Anaethesia and Administration of Bikunin into Tracheal Airway

Animals were anaesthetised using halothane. Once a satisfactory level of anaesthesia was induced a small incision was made below the lower jaw. The trachea was exposed and 100 μl volume of vehicle, bikunin (0.5 ug or 5 ug) or amiloride (100 uM) was instilled onto the tracheal surface using a needle and syringe. Once injected, the skin incision was sealed using Vetbond® (cyanocacrylate tissue glue). The animals were then allowed to recover.

Preparation of Guinea-Pig for Measurement of Tracheal Potential Difference

Two hours following agent treatment, guinea-pigs were anaesthetised for a second time with Hypnom® and Hypnovel® and immobilised in a supine position. Rectal temperature, measured with a thermistor probe was maintained at 37° C. by manual adjustment of a heat lamp. A ventral midline incision was made from the lower jaw to the clavicles. Using blunt dissection a length of trachea was exposed and bisected at the upper edge of the sternum. The external jugular vein was exposed and cannulated. The caudal part of the trachea was then cannulated to allow the animal to spontaneously breath room air. The animal was then placed supine and its body temperature maintained using the heat lamp. 20 min. following induction of i.m. anaesthesia the tracheal agar electrode was inserted into the cephalad trachea and tracheal potential difference was measured for 60 minutes. The reference electrode was placed under cephalad trachea in contact with the trachea cartilage. The wound site was covered to prevent drying.

Results

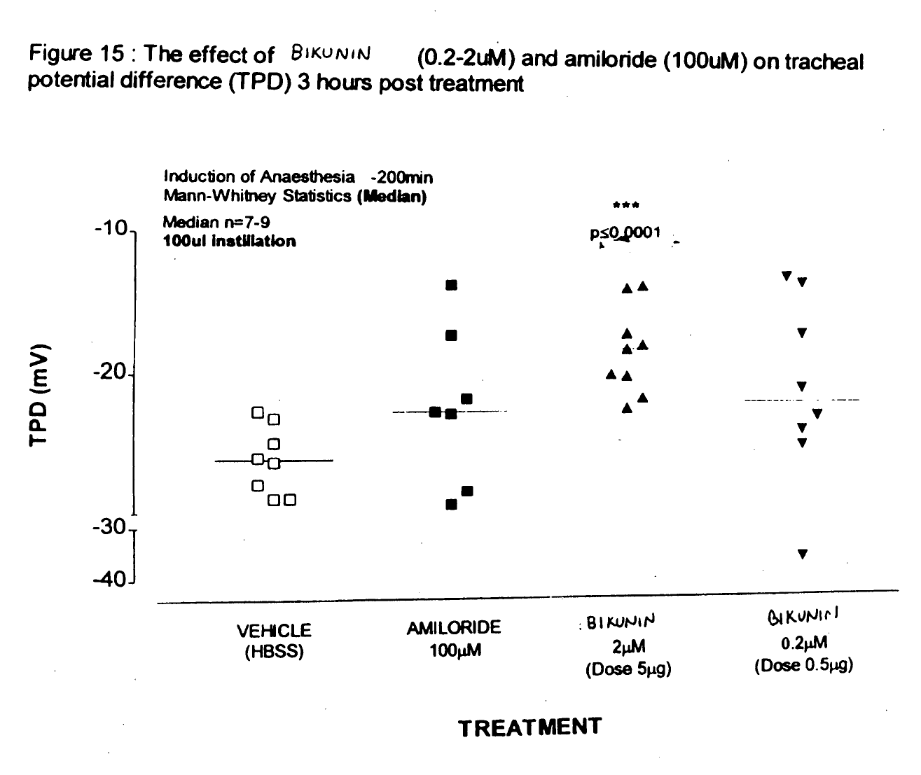

As shown in FIG. 15, Bikunin (5 ug) inhibited the potential difference in guinea pig trachea in vivo following three hours of treatment relative to vehicle. The effect of Amiloride (100 uM) and Bikunin (0.5 ug) is shown for comparison.

### EXAMPLE 12

The Effect of Bikunin on Tracheal Mucus Velocity in the Guinea-Pig

The aim of this study was to investigate the effect of the Kunitz family serine protease inhibitor Bikunin on guinea-pig tracheal mucus velocity 1.5 hours post treatment. This agent was delivered into the cephalad trachea by topical instillation. TMV was monitored 1.5 hours later for 60 mins. The procedure used in this Example is described in Newton et al. in “Cila, Mucus and Mucociliary Interactions,” Ed., Baum, G. L. et al., Marcel Dekker, New York, 1998; Newton et al., Ped. Pulm. S17, Abs. 364, 1998).

Materials and Methods/Reagents Used:

A Bikunin (1-170) formulation (50 ug/mL Bikunin (SEQ ID NO: 52) (as described in Example 17 below) was prepared in HBBS containing 137 mM NaCl, 3 mM KCl, 3 mM KH₂PO₄, 8 mM Na₂HPO₄, 0.2% Tween-80, pH 7.1). The formulation was sterile filtered and endotoxin tested prior to use in this example. HBSS was used as a control solution. Hypnorm® (Fentanyl citrate 0.315 mg/mL and Fluanisone 10 mg/mL) was obtained from Janssen Animal Health and Hypnovel® (Midazolam 5 mg/mL) was obtained from Roche. Male Dunkin-Hartley guinea pigs (550-750 g) were supplied by David Hall, UK. Thermistor probes were obtained from Kane-May Ltd, UK.

Induction of Anaethesia and Administration of Bikunin into Tracheal Airway

Animals were anaesthetized using halothane. Once a satisfactory level of anaesthesia was induced, a small incision was made below the lower jaw. The trachea was exposed and 100 ul volume of vehicle or bikunin (5 ug) was instilled onto the tracheal surface using a needle and syringe. Once instilled, the skin incision was sealed using Vetbond® (cyanocacrylate tissue glue). The animals were then allowed to recover.

Measurement of Tracheal Mucus Velocity (TMV)

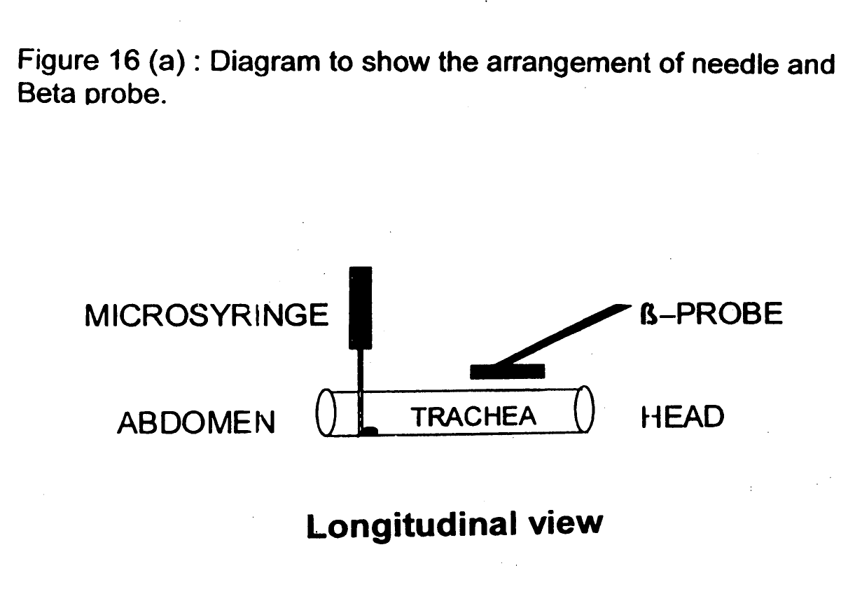

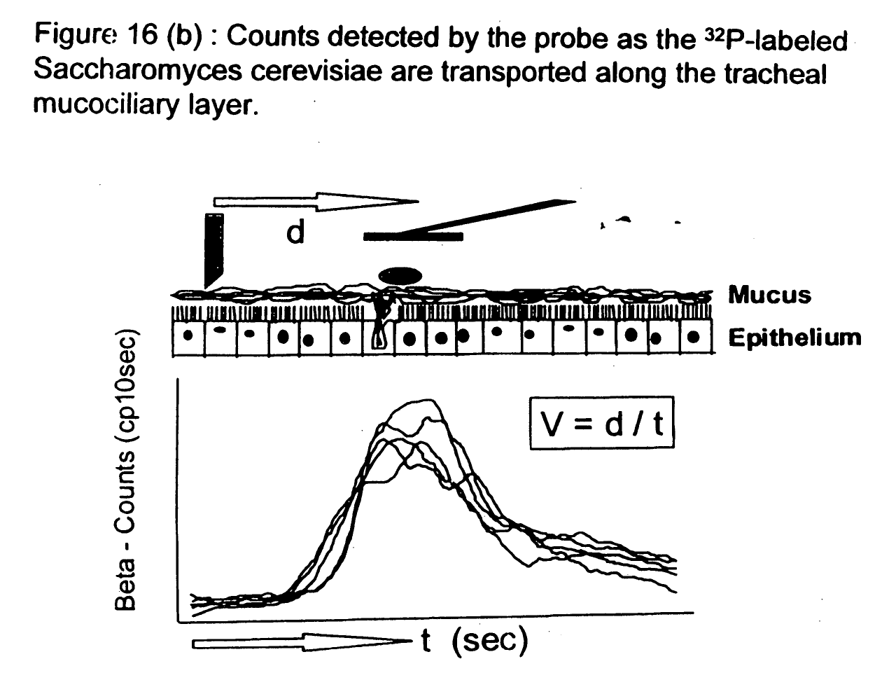

TMV was monitored using a lead collimated miniature Beta particle detector probe arranged to detect the radioactivity emitted from an injected aliquot of ³²P-labelled Saccharomyces cerevisiae as it was transported on the tracheal mucociliary layer of an anaesthetized guinea pig (Newton and Hall 1998) FIG. 16(a) illustrates the arrangement of the syringe and beta probe. FIG. 16(b) illustrates the counts detected by the probe as the ³²P-labelled S. cerevisiae is transported along the tracheal mucociliary layer.

70 minutes following instillation of bikunin, each animal was anaesthetized for a second time using Hyponorm® and Hyponovel® and immobilized in a supine position. The first TMV measurement was made 20 minutes afterwards. Subsequent measurements were taken every 15 minutes. The procedure for TMV measurements is described, in detail, in Newton et al., “Cilia. Mucus and Mucociliary Interactions.” Ed. Baum, G. L., Preil, Z., Roth, Y., Liron., Ostfield, E., Marcel Dekker. New York, 1990 and Newton et al. in Pediatric Pulmonology S17, Abs 364, 1998.

Results

As shown in FIG. 16(c), Bikunin (5 ug) increased TMV in vivo in guinea pig, relative to saline, over a sustained period of 1.5 to 2.5 hours following administration.

### EXAMPLE 13

Bikunin Decreases Sodium Current in Cultured Human Bronchial Epithelial (HBE) Cell Short Circuit Current (Isc) In Vitro.

Tertiary HBE cell monolayer grown to confluence were mounted in modified Ussing chambers, immersed in Krebs buffer (KBR) solution and bubbled with 95% O₂/5% CO₂ warmed to 37 C.

Cells were left to equilibrate for 20 minutes before calibrating for background noise and fluid resistance. Transepithelial potential difference was then clamped to 0 mV using a WPI EVC 4000 voltage clamp. Ag/AgCl electrodes were used to monitor Isc. Once a stable baseline was achieved (typically 10-20 min), cells were treated with amiloride (10 uM). Once a response to amiloride was seen, it was washed out with KBR solution. After return to baseline and equilibration, Bikunin (1-170) (as described in Example 17 below) (0.5-50 ug/mL in PBS) or PBS control was added. 90 minutes following agent treatment, amiloride (10 uM) was added. Once the current was stable forskolin (10 uM) and then bumetanide (100 uM) was added.

Results

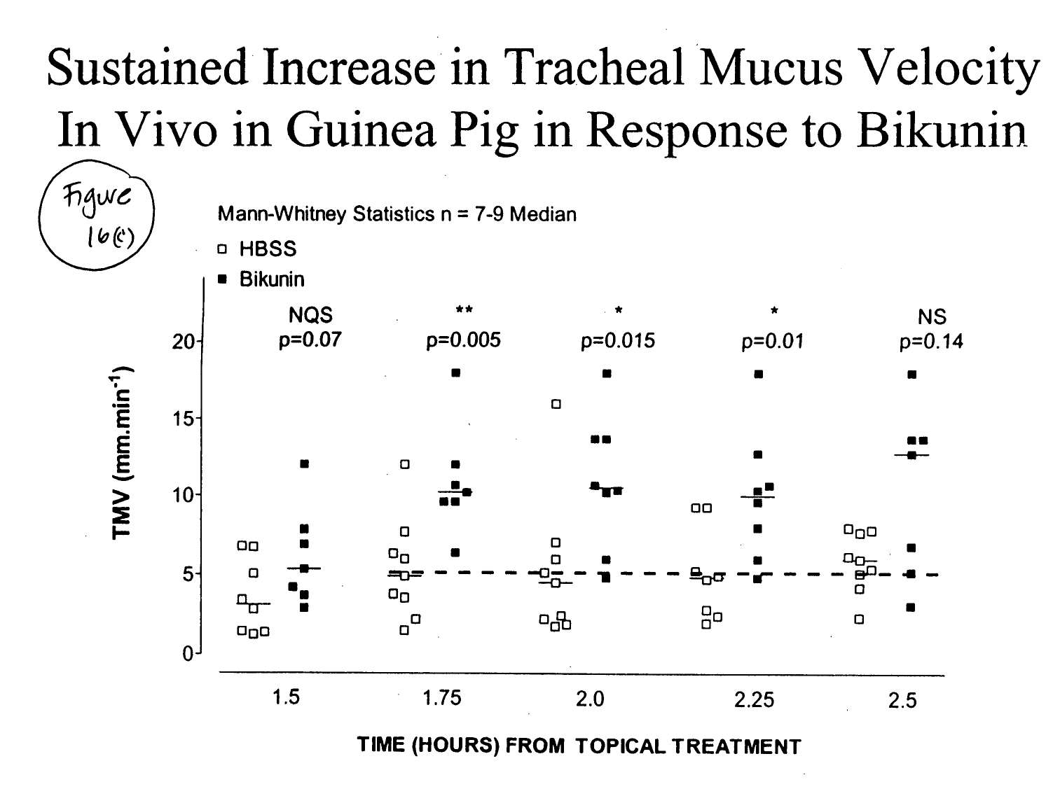

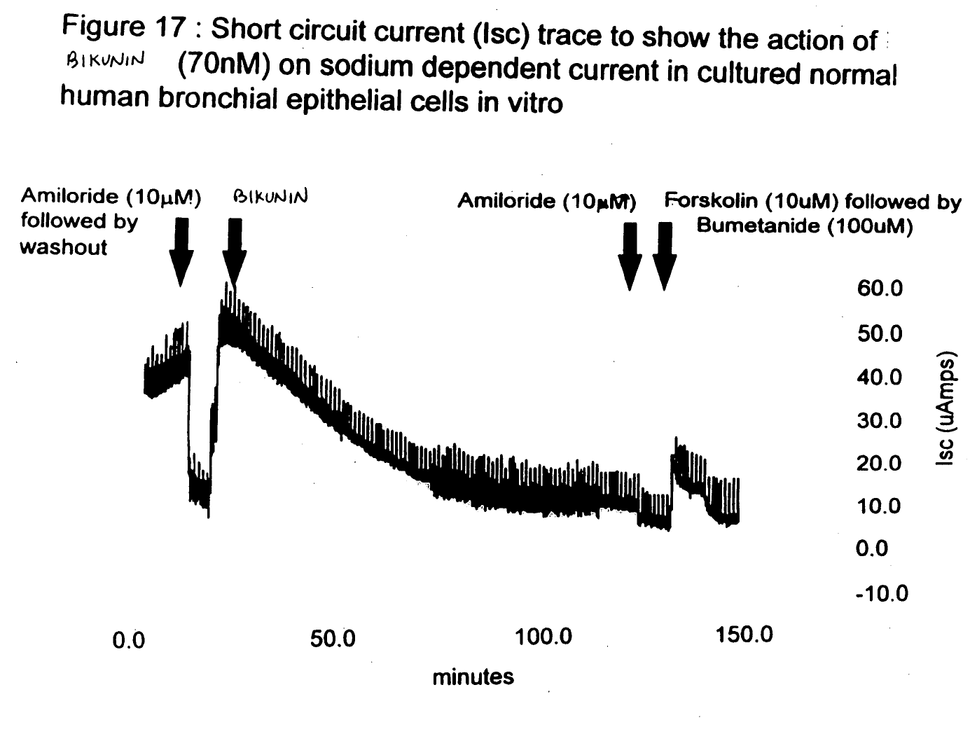

As shown in FIG. 17, Bukinin (70 nM) inhibited sodium current in vitro in human bronchial epithelial cells over a 90 minute period. Forskolin induced cAMP-mediated chloride secretion and monolayer resistance was unaffected.

### EXAMPLE 14

The effect of Hypertonic Saline (14.4%) on TMV in the Guinea Pig

The aim of this comparative study was to investigate the effect of hypertonic saline (14.4%×5 min) on guinea-pig tracheal mucus velocity. This agent was delivered into the cephalad trachea by aerosol. TMV was monitored immediately and every 15 minutes for 30 minutes. The procedure used in this Example is described in Newton et al. in “Cila, Mucus and Mucociliary Interactions,” Ed., Baum, G. L. et al., Marcel Dekker, New York, 1998; Newton et al., Ped. Pulm. S17, Abs. 364, 1998).

Materials and Methods/Reagents Used

Hypnorm® (Fentanyl citrate 0.315 mg/mL and Fluanisone 10 mg/mL) was obtained from Janssen Animal Health and Hypnovel® (Midazolam 5 mg/mL) was obtained from Roche. Male Dunkin-Hartley guinea pigs (550-750 g) were supplied by Harlan UK Ltd. Thermistor probes were obtained from Kane-May Ltd, UK.

Measurement of Tracheal Mucus Velocity:

Animals were anaesthetized using Hypnorm® and Hypnovel®. TMV was monitored using a lead collimated miniature Beta particle detector probe arranged to detect the radioactivity emitted from an injected aliquot of ³²P-labelled Saccharomyces cerevisiae as it was transported on the tracheal mucociliary layer of an anaesthetized guinea pig (Newton and Hall 1998)

The first TMV measurement (run 1) was made 20 minutes after administration. Subsequent measurements were taken every 15 minutes. At a time point 6 minutes before the second run, a 5 minute aerosol of saline (0.9%) or hypertonic saline (14.4%) was administered. The radiolabelled tracer particles were given via the 0.5 um hole made in the trachae. An aerosol of ether saline (0.9%) or hypertonic saline (14.4%) was generated by a Pari pressure nebulizer. The aerosol was switched off one minute before the second run. The procedure for TMV measurements is described, in detail, in Newton et al., “Cilia, Mucus and Mucociliary Interactions.” Ed. Baum, G. L., Preil, Z., Roth, Y., Liron, Ostfield, E., Marcel Dekker. New York, 1990 and Newton et al. in Pediatric Pulmonology S17, Abs 364, 1998.

Results

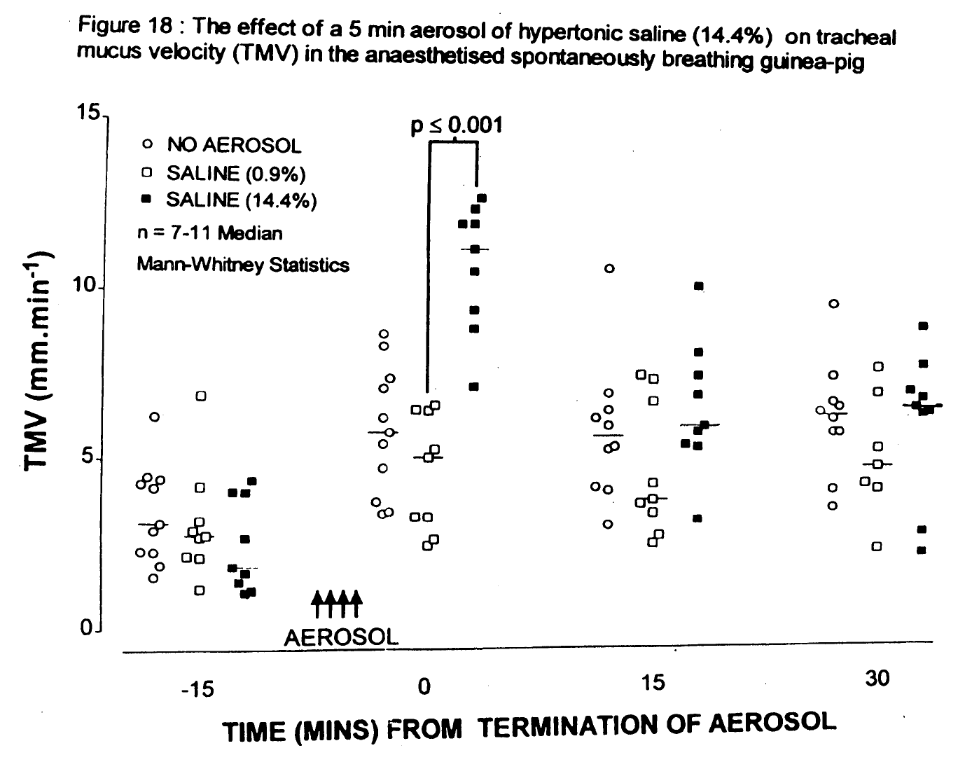

As shown in FIG. 18, hypertonic saline (14.4%×5 mins) caused a transient increase in TMV immediately after aerosol.

### EXAMPLE 15

Effect of Amiloride on TMV in the Guinea-Pig

The aim of this study was to investigate the effect of amiloride (10 mM×20 min.) on guinea-pig tracheal mucus in the anaesthetized spontaneously breathing guinea pig. This agent was delivered into the cephalad trachea by aerosolization as described in Example 14. The TMV measurement procedure used in this Example is described in Newton et al. in “Cilia, Mucus and Mucociliary Interactions,” Ed., Baum, G. L. et al., Marcel Dekker, New York, 1998; Newton et al., Ped. Pulm. S17, Abs. 364, 1998).

Materials and Methods/Reagents Used

An amiloride formulation (10 mM) in water was prepared for this example. Hypnorm® (Fentanyl citrate 0.315 mg/mL and Fluanisone 10 mg/mL) was obtained from Janssen Animal Health and Hypnovel® (Midazolam 5 mg/mL) was obtained from Roche. Male Dunkin-Hartley guinea pigs (550-750 g) were supplied by Harlan UK Ltd. Thermistor probes were obtained from Kane-May Ltd, UK.

Measurement of Trachael Mucus Velocity

Animals were anaesthetized using Hypnorm® and Hypnovel®. TMV was monitored using a lead collimated miniature Beta particle detector probe arranged to detect the radioactivity emitted from an injected aliquot of ³²P-labelled Saccharomyces cerevisiae as it was transported on the tracheal mucociliary layer of an anaesthetized guinea pig. Guinea pigs were anaesthetized with Hypnorm® and Hyponovel at time 0. AmiIoride (10 mM×20 min) was administered by aerosol. The first TMV measurement was made immediately afterwards and subsequent measurements were taken every 15 minutes.

Results

As shown in FIG. 19, amiloride (10 mM×20 mins) caused a statistically significant increase in TMV 15 minutes after aerosol.

### EXAMPLE 16

Aprotinin Double Mutein Decreases Sodium Current in Cultured Human Bronchial Epithelial (HBE) Cell Short Circuit Current (Isc)

The aim of this study was to investigate the effect of the Kunitz family serine protease inhibitor Aprotinin double mutein on Isc in vitro. Tertiary HBE cell monolayers grown to confluence were mounted in modified Ussing chambers, immersed in Krebs buffer (KBR) solution and bubbled with 95% O₂/5% CO₂ warmed to 37 C. Aprotinin double mutein is Des Pro2-Ser10-Arg15-Asp24-Thr26-Glu31-Asn41-Glu53-Aprotinin which is described in Example 1 of EP 821 007, published Jan. 28, 1998, incorporated by reference in its entirety.

Cells were left to equilibrate for 20 minutes before calibrating for background noise and fluid resistance. Transepithelial potential difference was then clamped to 0 mV using a WPI EVC 4000 voltage clamp. Ag/AgCI electrodes were used to monitor Isc. Once a stable baseline was achieved (typically 10-20 mins), cells were treated with a amiloride (10 uM). Once a response to amiloride was seen, it was washed out with KBR solution. After return to baseline and equilibration, Bikunin (5 ug/mL), Aprotinin double mutein (0.5 to 5 ug/mL), Aprotinin (1.5 to 5 ug/mL) or PBS was added. 90 minutes following agent treatment, amiloride (10 uM) was added.

Results

As shown in FIG. 20, Aprotinin double mutein (0.5 to 5 ug/mL) dose dependently inhibited sodium current in vitro in human bronchial epithelial cells over a 90 minute period.

### EXAMPLE 17

Expression, Purification and Comparative Protease Inhibitory Activity of Placental Bikunin (1-170) Expressed in Chinese Hamster Ovary (CHO) Cells

(a) Development of Stable, High-Producing CHO Cell Lines that Express Bikunin

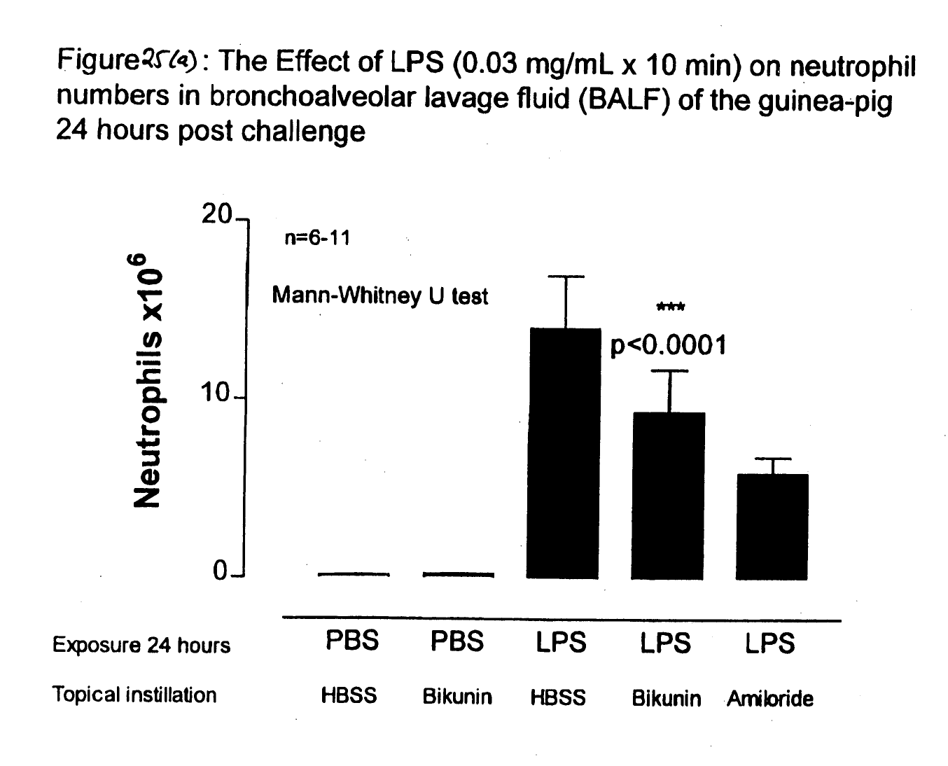

Stable production cell lines that secrete high quantities of bikunin were developed by transfecting CHO (dhfr-) cells with the expression vector shown in FIG. 27. The vector was constructed using standard recombinant DNA techniques. A description of the construction of the expression vector and CHO cell expression system can be found in U.S. Ser. No. 09/______, filed Nov. 12, 1999, entitled “Method of Producing Glycosylated Bikunin,” by Inventor Sam Chan. Briefly, the expression vector pBC-BK was constructed by cloning bikunin cDNA immediately downstream of the cytomegalovirus immediate early promoter and upstream of the polyadenylation signal sequence. The expression vector pBC-BK consists of a transcriptional unit for bikunin, dihydrofolate reductase, and ampicillin resistance. Bikunin cDNA was released from the cloning vector by restriction enzymes, blunt-ended, and ligated to linearized pBC. The linearization of pBC was done by a single restriction enzyme digestion. The orientation of bikunin cDNA was confirmed by sequencing.

About 1×10⁶ CHO (Chinese hamster ovary) cells were transfected with 10 μg of pBC-BK using Lipofectin reagents (Life Technology, Bethesda, Md.) according to manufacturer's instructions. The cells were then selected in the presence of 50 nM methotrexate and grown in DME/F12 media deficient in thymidine and hypoxanthine plus 5% dialyzed fetal bovine serum. Cell populations were screened for bikunin production with a chromogenic assay. Briefly, bikunin standards or culture fluid was serially diluted and incubated with an equal volume of kallikrein at 37° C. for 30 minutes after which a chromogenic substrate, N-benzoyl-Pro-Phe-Arg-pNA, was added. The reaction was incubated for 15 minutes before the addition of 50% acetic acid. The amount of p-nitroanilide released was measured at 405 nM. The high producing populations were further selected in media containing increasing concentrations of methotrexate (100 to 400 nM methotrexate) and screened for the production of bikunin. Limiting dilution cloning was then applied to derive clones with high and stable productivity. The cloning was done in the absence of methotrexate using standard tissue culture techniques by depositing 1 cell/well in 96-well plates. A clone designated FD3-1 was chosen for productivity evaluation in a bioreactor and was deposited on Nov. 12, 1999 with the American Type Culture Collection (ATCC), Rockville, Md., and was assigned accession number

(b) Serum-Free Production of Bikunin in a Perfusion Bioreactor

Continuous production of bikunin was done by continuous perfusion fermentation. A 1.5 liter Wheaton fermenter was inoculated with a stable CHO cell line at 2×10⁶ cells/ml and perfused at a medium exchange rate of 0.5 liters/day. The production medium was a DME/F12-based medium supplemented with insulin (10 μg/ml) and FeSO₄.EDTA(50 μM). The cell density was maintained at 4×10⁶ cells/mi. The average daily yield of the fermenter was −20 mg/day. The production of bikunin was stably maintained for 21 days.

(c) Purification of Bikunin (1-170) Produced from a CHO Cell Expression System

Bikunin produced from CHO cells was purified using standard chromatography techniques involving ion exchange, metal chelate, and size exclusion chromatography as outlined in FIG. 29.

The SP column (18×10 cm, 2.5 L) was prepared with SP-Sepharose Fast Flow (Pharmacia), and equilibrated. Cold filtered CHO cell harvest (TCF) was diluted 1:2.5 with cold sterile water, and the pH was adjusted to 5.0. Chromatography was performed at ambient temperature with cold buffers. The cold starting material was loaded on the column at 800 mL/min (189 cm/hr). The amount of bikunin loaded onto the column ranged from 0.888-1.938 g (approximately 14 mg/L). After loading, the column was washed with equilibration buffer and the bikunin eluted with elution buffer. The eluate was collected at 2-8° C. (in an ice bath) and immediately adjusted to pH 7 with 6 N NaOH. The column was washed, then sanitized with cold (2-8° C.) 1 N NaOH, and stored at 2-8° C. in 20% ethanol until its next use. The equilibration and wash buffer contained 50 mM NaCl, 30 mM NaH₂PO₄, pH 5.0; the elution buffer contained 350 mM NaCl, 30 mM NaH₂PO₄, pH 5.0; and the pH adjusting buffer was 1 M citric acid, 1 M NaH₂PO₄, pH 2.4.

Thawed SP-Sepharose eluate was concentrated by ultrafiltration (UF) approximately 10-fold to reduce the volume before a 5 to 7-fold diafiltration (DF) was performed in preparation for anion exchange chromatography. All operations were performed at ambient temperature in a horizontal flow hood. UF/DF utilized a Pellicon 2 “mini” filter system from Millipore (Bedford, Mass.) and two 10 kDa regenerated cellulose cartridges (P2C010C01). Flux rates were approximately 130±20 mL/min for the two-cartridge system and were maintained by regulating the inlet and outlet pressures between 22 to 26 psi and 12 to 16 psi respectively. Circulation was with a peristaltic pump; recirculation was set to 500 to 600 mL per minute before transmembrane pressure adjustment. Diafiltration was performed with cold 10 mM NaH₂PO₄ buffer, pH 8.1.

The Q-Sepharose column chromatography was performed as follows. A 13×9 cm, 1.2 L column of Q-Sepharose Fast Flow (Pharmacia) was washed with 5 column volumes (CV) of sterile water and equilibrated with approximately 10 CV's equilibration buffer. Diafiltered SP eluate was adjusted to pH 8.1 and applied on the Q-Sepharose column at 100 mL/min (45 cm/hr). The amount of bikunin loaded onto the column ranged from 1121-2607 mg (approximately 15 mg/mL). After loading, the column was washed with equilibration buffer until the UV absorbance at A280 reached baseline; then the bikunin was eluted. The eluate was collected and used as feed material for Zn-IMAC, zinc immobilized metal ion adsorption chromatography. The column was cleaned with 1 M NaOH, rinsed with sterile water, and stored in 20% ethanol. All operations were performed at 2-8° C. Equilibration and washing buffer for Q-Sepharose column contained 10 mM NaH₂PO₄, pH 8.1. The elution buffer contained 10 mM NaH₂PO₄, 100 mM NaCl, pH 8.1.

A Zn-IMAC column (5×10 cm) containing approximately 200 mL bed volume of Chelating Sepharose Fast Flow (Pharmacia) was charged with 3 volumes of ZnSO₄ solution (see below); washed with 2 volumes of sterile water, and equilibrated with 6 volumes of buffer as described below. The Q-Sepharose eluate was adjusted to pH 7.4 and 300 mM NaCl (by addition of NaCl solid), applied to Zn-IMAC at 30 mL/min (92 cm/hr linear rate), and then the column was washed with equilibration buffer until the UV absorbance reached baseline. The amount of bikunin loaded onto the column ranged from 0.097-1.681 g (approximately 0.63 mg/mL). The flowthrough and wash were collected for UF. The column was stripped, and sanitized with 0.5 M NaOH. All operations of this step were performed at 2-8° C. Equilibration buffer for Zn-IMAC contained 10 mM NaH₂PO₄, 300 mM NaCl, pH 7.4; the strip buffer contained 50 mM EDTA, 10 mM NaH₂PO₄, 300 mM NaCl, pH 7.4; charging solution, 2 mg/mL ZnSO₄ □7H₂O.

The Zn-IMAC flow-through was concentrated by ultrafiltration 5-fold with the Pellicon 2 system (Millipore) for Sephacryl S-200 chromatography. Permeate flux rates were approximately 60 to 70 mL/min and were maintained as described earlier. Recirculation was at 400 to 500 mL/min.

A column (10×58.5 cm) containing 4.59 L of Sephacryl S-200 High Resolution (Pharmacia) was equilibrated with 137 mM NaCl, 2.7 mM KCl, 2.9 mM KH2PO4, 10 mM NaH2PO4, 8 mM Na2HPO4, 2 mg/L Tween 80, pH 7.2. Flow rate was 30 mL/min (23 cm/hr). In a typical run, 1475 mg of bikunin in a volume of 95 mL was applied to the column.

The post-S-200 pool was treated with Etox resin in batch mode for removal of any potential pyrogen. ActiClean Etox, 2705 resin (Sterogene Bioseparations, Inc.) was rinsed with 1 M NaOH and incubated for 5 hours in 1 M NaOH at room temperature with agitation. The resin was washed and equilibrated with PBS, pH 7.2. Eighty mL of the filtered and equilibrated resin was added to 1063 mL of bikunin S200 pool (5460.11 mg), and agitated overnight at 2-8° C. The ETOX resin was then removed by filtration with 0.2 micron Nalgene flask filter.

Results. Table 10 shows the average yield afforded by each step.

TABLE 10

| Purification Step | Average Yield (%) |
|---|---|
| SP-Sepharose | 88.1 |
| UF/DF plus filtration | 81 |
| Q-Sepharose | 59 ± 14 |
| Zn-IMAC | 99.5 |
| Sephacryl S-200 | 81 |
| ETOX Resin | 93 |

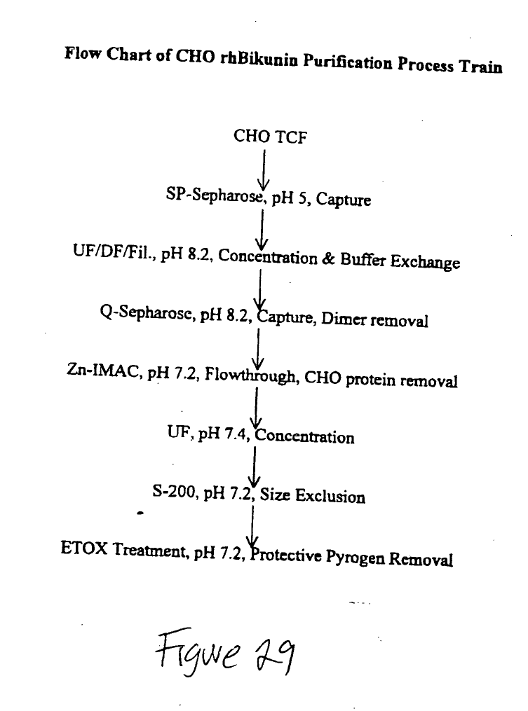

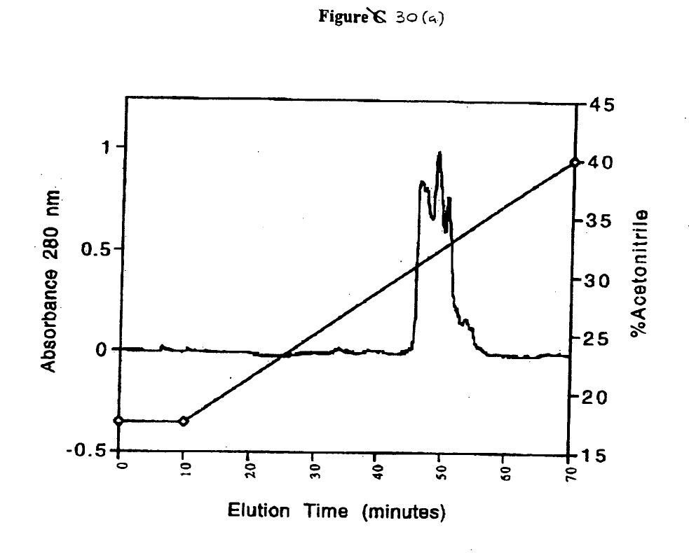

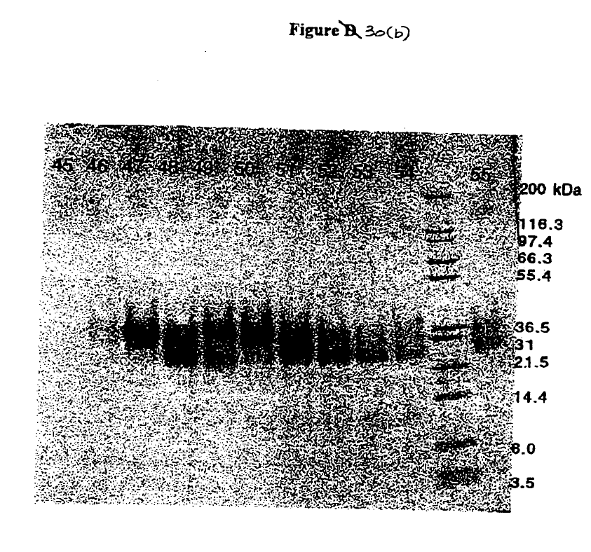

Overall yield of bikunin was about 30% with a purity of 95%. Mass spectroscopy data also suggested that in addition to full length Bikunin (1-170) molecules, species lacking three (G-S-K) and four (L-G-S-K) amino acids from the carboxy end of Bikunin (1-170) were present in the pure protein pool. The material produced was shown to be stable to degradation when exposed to 72-hours incubation at ambient temperature or at 37° C., neutral pH. N-terminal sequencing, gel electrophoresis, immunoblotting, and amino acid analysis indicated that the bikunin was substantially pure (no other sequences were detected). An additional reverse phase chromatography step revealed that the CHO-derived purified bikunin was still able to be fractionated into several species (FIG. 30A). CHO bikunin (8.5 mg) was adjusted to pH 2.5 with trifluoroacetic acid (TFA, 0.1% final concentration) and subjected to chromatography on a C18 reverse-phase column (Vydac, 2.5×25 cm) equilibrated in 17.5% acetonitrile and 0.1% TFA at a flow rate of 2 ml/min. CHO bikunin was eluted with a linear gradient of 17.5-40% acetonitrile in 0.1% TFA over 60 min. FIG. 30B shows the silver stained SDS-PAGE profile of these fractions (lane between 54 and 55 represents molecular size markers).

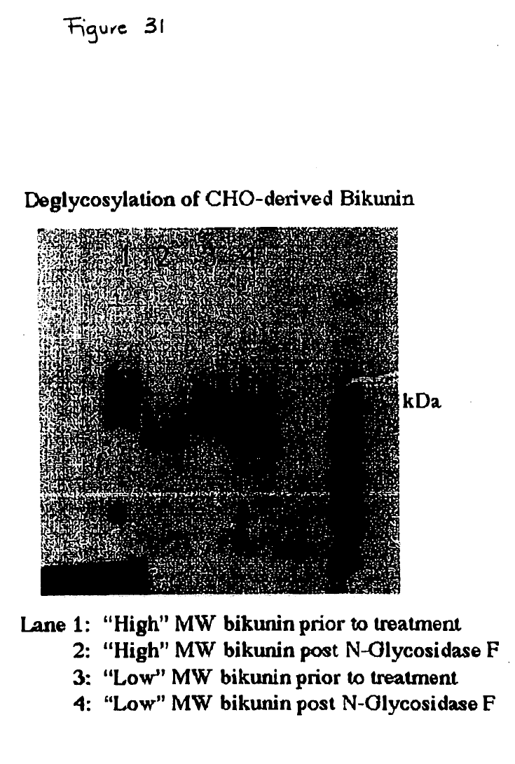

Preliminary carbohydrate analysis was performed on the glycosylated isoforms of CHO bikunin having a MW ranging from about 21 kDa to about 38 kDa. The total sugar content was found to be 7.5%. Both N-linked sites (Asn-30 and Asn-61) were found to be occupied with carbohydrate structures. Chromatographic and mass spectrometric analysis confirmed the presence of very heterogeneous and highly branched oligosaccharide structures contributing to the size heterogeneity observed for the purified bikunin. About 90% of the oligosaccharides were sialylated and the remaining structures were neutral. When treated with N-Glycosidase F, the glycosylated isoforms of CHO bikunin (FIG. 30B) were converted to a single 18 kDa isoform (See FIG. 31).

The sialic acid content of bikunin was analyzed by incubation with sialidase in 50 mM sodium acetate buffer, pH 5.0, for 18 hours in a capped microfuge tube. Sialic acids were separated on a Carbo Pac PA1 anion-exchange column using a buffer gradient of 20-250 mM sodium acetate in 100 mM NaOH for 50 minutes at a flow rate of 1 ml/min. Detection was done with a pulsed electrochemical detector and quantitated by comparing retention times and peak areas of samples to standard sialic acids (N-acetylneuraminic acid and N-glutarylneuraminic acid). The results are shown in Table 11.

TABLE 11
Sialic acid composition of bikunin

| Sialic acid | Contents (g/100 g of bikunin) |
|---|---|
| N-Acetylneuraminic acid | 5.4 |
| N-Glutarylneuraminic acid | 0 |

### EXAMPLE 18

Comparative Protease Inhibitor Activity of Placental Bikunin (1-170) Expressed in Chinese Hamster Ovary (CHO) Cells

General. The in vitro specificity of recombinant bikunin was determined using the materials and methods as described in Examples 3, 4, 7, and 10. Table 12 below shows the efficacy of recombinant bikunin as an inhibitor of various serine proteases in vitro. Data is shown using either recombinant bikunin or aprotinin.

Proteases. Human plasmin and human plasma kallikrein quantitation was carried out by active site titration using p-nitrophenyl p′-guanidinobenzoate HCl as previously described (Chase, T., and Shaw, E., (1970) Methods Enzmol. 19, 20-27). Human tissue kallikrein (Bayer, Germany) was quantitated by active site titration using bovine aprotinin as a standard and PFR-AMC as a substrate assuming a 1:1 complex formation. The Kₘ for GPK-AMC with plasmin under the conditions used was 726 μM; the Kₘ for PFR-AMC with human plasma kallikrein was 457 μM; the Kₘ for PFR-AMC with human tissue kallikrein was 5.7 μM.

Inhibition Kinetics: The inhibition of human plasmin by CHO expressed placental bikunin (1-170) and aprotinin was determined with plasmin (50 pM) and CHO expressed placental bikunin (1-170) (0-2 nM) or aprotinin (0-4 nM) in buffer containing 50 mM Tris-HCl (pH 7.5), 0.1 M NaCl, and 0.02% triton x-100. After 30 min. incubation at 37° C., 25 μl of 20 mM GPK-AMC was added and the change in fluorescence monitored. The inhibition of human plasma kallikrein by CHO expressed placental bikunin (1-170) or aprotinin was determined using kallikrein (0.2 nM) and CHO expressed placental bikunin (1-170) (0-4 nM) or aprotinin (0-45 nM) in 50 mM Tris-HCl (pH 8.0), 50 mM NaCl, and 0.02% triton x-100. After 30 min. at 37° C., 5 μl of 20 mM PFR-AMC was added and the change in fluorescence monitored.

The inhibition of human tissue kallikrein by aprotinin or CHO expressed placental bikunin (1-170) was measured by the incubation of 0.35 nM human tissue kallikrein with CHO expressed placental bikunin (1-170) (0-10 nM) or aprotinin (0-0.5 nM) in a 1 ml reaction volume containing 50 mM Tris-HCl buffer pH 9.0, 50 mM NaCl, and 0.1% triton x-100. After 5 min. at 37° C., 5 ul of 2 mM PFR-AMC was added achieving 10 uM final concentration and the change in fluorescence monitored.

The inhibition of Factor XIa (from Enzyme Research Labs, South Bend, Ind.) was measured by incubating FXIa (0.1 nM) with either 0 to 40 nM CHO expressed placental bikunin (1-170) or 0 to 4 uM aprotinin in buffer containing 50 mM Hepes pH 7.5, 100 mM NaCl, 2 mM CaCl2, 0.01% triton x-100, and 1% BSA in a total volume of 1 ml. After 30 min at 37 C, 10 uI of 40 mM Boc-Glu(OBzl)-Ala-Arg-AMC (Bachem Biosciences, King of Prussia, Pa.) was added and the change in fluorescence monitored.

The apparent inhibition constant Kᵢ* was determined using the nonlinear regression data analysis program Enzfitter software (Biosoft, Cambridge, UK): The kinetic data from each experiment were analyzed in terms of the equation for a tight binding inhibitor:
Vᵢ/V₀=1−(E₀+I₀+Kᵢ*−[(E₀+I₀+Kᵢ*)²−4E₀1₀]^{1/2})/2E₀ (2)
where Vᵢ/Vₒ is the fractional enzyme activity (inhibited vs. uninhibited rate), and Eo and Io are the total concentrations of enzyme and inhibitor, respectively. Ki values were obtained by correcting for the effect of substrate according to the equation:
Kᵢ=Kᵢ*/(1+[S₀]/Kₘ) (3)
(Boudier, C., and Bieth, J. G., (1989) Biochim Biophys Acta. 995: 36-41).

Results: The Kᵢ values are listed in Table 12 below.

TABLE 12
Comparison of Ki values for the inhibition of various proteases by CHO
Bikunin (1-170) or aprotinin

| | CHO Bikunin (1-170) | |
|---|---|---|
| Protease | Ki (nM) | Aprotinin Ki (nM) |
| Human plasma kallikrein | 0.5 | 23.0 |
| Human tissue kallikrein | 0.3 | 0.004 |
| Human plasmin | 0.2 | 0.2 |
| Human FXIa | 1.9 | 270.0 |

The results show that recombinant bikunin can be expressed in CHO cells to yield an active protease inhibitor that is effective against at least three different serine proteases. Recombinant bikunin was more potent than aprotinin against human plasma kallikrein, and human FXIa. It was equipotent with aprotinin at inhibiting human plasmin.

### EXAMPLE 19

Aprotinin Decreases Sodium Current in Cultured Human Cystic Fibrosis Bronchial Epithelial Cell Short Circuit Current (Isc) In Vitro

HBE cells were isolated from CF patient lung transplant tissue and grown in collagen coated flasks for one week. The cells were then passaged and seeded onto collagen coated Costar Transwell filters (0.33 cm2) and grown in DMEM/F12 media supplemented with 2% Ultroser G. Cells were grown at an air liquid interface and used 2 to 4 weeks after seeding:

Cells on the Transwell filters were mounted in modified Costar Ussing chambers and studied under Isc conditions. Baseline Isc values were recorded (0 to 10 minutes), and then aprotinin (1 mg/mL in PBS) was added to the apical side. Isc was recorded for 100 minutes and then the apical bath fluid was exchanged with fresh buffer. Trypsin (100 BAEE units/mL) was added to the apical side and Isc recorded for 10 minutes. Amiloride (10 uM) was then added to the apical side and Isc was recorded for a further 10 minutes.

Results

As shown in FIG. 21, aprotinin (1 mg/mL in PBS) inhibited Isc in vitro in human CF bronchial epithelial cells over a 100 minute period. After washout, Isc was increased by treatment of the apical surface with the serine protease, trypsin. Finally, addition of amiloride (10 uM) demonstrated that the changes in Isc were the result of changes in sodium dependent current.

### EXAMPLE 20

Assessment of Activity of Bikunin (1-170) Following Nebulization

The aim of this study was to assess the anti-protease activity of Bikunin (1-170) described in Example 17 following nebulization. All studies were performed with Bikunin formulated in phosphate buffered saline, pH 7.4 (137 mM NaCl, 3 mM KCl, 3 mM KH₂PO₄, 8 mM Na₂HPO₄, 0.02 g/L Tween 80).

Methods of Nebulization

Raindrop® Medication Nebulizer: Bikunin was aerosolized at concentrations of 1 and 3 mg/mL. The nebulizer cup was filled with 2.5 mL of Bikunin solution. Aerosolisation was performed at 7.35 L/min or 35 psi.

Collison Nebulizer: Bikunin was aerosolised at a concentration of 4.7 mg/mL. The nebulizer cup was filled with 2.5 mL of Bikunin solution. Aerosolisation was performed at 35 psi.

Collection of Nebulised Samples

Aerosolised Bikunin was collected using a twin impinger (6.4 um mean aerodynamic particle cut-off size at 60 L/min through the system). The impinger works on the principle of liquid impingement and divided the aerosol into a non-respirable fraction (>6.4 um collected in Stage 1) and a respirable fraction (<6.4 um collected in Stage 2).

Measurement of Anti-Protease Activity

Bikunin activity was measured in vitro by its inhibition of human plasma kallikrein.

Results

Raindrop® Medication Nebulizer: Activity (Ki) values for the pre- and post-nebulization samples were as follows: 1 mg/mL Bikunin: Ki values were 0.47 (±0.02) and 0.76 (±0.04) respectively; and for 3 mg/mL Bikunin (1-170) the Ki values were 0.52 (±0.03) and 0.62 (±0.03) respectively.

Collison Nebulizer: Activity (Ki) values for the pre- and post-nebulization samples were 0.27 (±0.03) and 0.45 (±0.03) respectively.

Conclusion:

Pre- and post-nebulized Bikunin samples from both the Raindrop and Collison nebulizers exhibited similar activities (similar Ki values within assay variability), indicating that Bikunin (1-170) was stable to aerosolization and retained its anti-protease activity following nebulization.

### EXAMPLE 21

The Effect of Bikunin on Tracheal Mucus Velocity in the Sheep

The aim of this study was to investigate the effect of the Kunitz family serine protease inhibitor Bikunin (1-170) described in Example 17 on sheep trachcal mucus velocity over 8 hours after treatment. This agent was delivered by nebulised aerosol administration to the airways. The procedure used in this example is described in O'Riordan et al., J. Applied Physiol. 85(3), 1086-1091, 1998.

Measurement of TMV

Adult ewes were restrained in an upright position, with their heads immobilized, in a specialized body harness. They were nasally intubated with an endotracheal tube, with the cuff placed just below the vocal cords. The inspired air was warmed and humidified. To minimize possible impairment of TMV caused by inflated cuffs, the endotracheal tube cuff remained deflated throughout the study except for the period of aerosol administration.

To measure TMV 5 to 10 radiopaque Teflon particles (approximately 1 mm in diameter, 0.8 mm thick, and weighing 1.5 to 2 mg) were insufflated into the trachea via a catheter placed within the endotracheal tube. The movement of the Teflon particles was then measured over a 1 minute period. The procedure used in this example is described in Russi et al., J. Applied Physiol. 59(5), 1416-1422, 1985. A collar containing radiopaque markers of known length was applied to the exterior of the animals and used as a standard to convert distance traversed by the particles on the video screen to actual distance traveled. TMV was calculated from the average distance in a cephalad direction traveled per minute for 5 to 10 Teflon particles. Baseline TMV was measured immediately prior to administration of aerosol.

Test substances; PBS, 1 mg/mL Bikunin in PBS, or 3 mg/mL Bikunin in PBS; were delivered to the sheep airways as an aerosol (3 mL) generated using a Raindrop jet nebuliser operated at a flow rate that produced droplets of mass median aerodynamic diameter of 3.6 um. TMV was measured immediately after administration of test substance (0 hours), then again at 0.5, 1, 2, 3, 4, 5, 6, 7, and at 8 hours.

Results

As shown in FIG. 22, 9 mg Bikunin aerosol (3 mL of 3 mg/mL) delivered to sheep airways significantly increased TMV at 0, 0.5, 3, 4, 5, 6, 7, and 8 hours compared to the same time points for a group of animals receiving PBS vehicle aerosol. At 24 hours TMV had returned to baseline rates in both the Bikunin treatment and in the PBS vehicle groups.

At a lower dose (3 mg Bikunin aerosol (3 mL of 1 mg/mL)), no significant differences were observed in TMV between treatment and vehicle groups at any time point studied.

### EXAMPLE 22

Bikunin (50 ug/mL) Decreases Sodium Current in Cultured Guinea Pig Tracheal Epithelial (GPTE) Cell Short Circuit Current (Isc)

The aim of this study was to investigate the effect of Bikunin (1-170) (See Example 17) on Isc on GPTE cells in vitro. GPTE cells were seeded onto 1.2 cm diameter, 0.4 um pore size Snapwell™ inserts (Costar UK). Cells were grown to confluence and mounted in modified Ussing chambers 2-4 days after placement on air-liquid interface. Inserts were immersed in Krebs buffer (KBR) solution and bubbled with 95% O₂/5% CO₂ warmed to 370 C.

After a 20 minute equilibration period, transepithelial potential difference was then clamped to 0 mV using a WPI EVC 4000 voltage clamp. Ag/AgCl electrodes were used to monitor Isc. Once a stable baseline was achieved (typically 20-30 mins), cells were treated with amiloride (30 uM). Once a response to amiloride was obtained, it was washed out with KBR solution. After return to baseline and equilibration, Bikunin (1-170) described in Example 17 (10 to 50 ug/mL) or PBS was added. 30 minutes following agent treatment amiloride (30 uM) was added.

Results

As shown in FIG. 23, Bikunin (1-170) (50 ug/mL) inhibited sodium current in vitro in guinea pig tracheal epithelial cells over a 30 minute period.

### EXAMPLE 23

Bikunin (1-170) (100 ug/mL) Decreases Sodium Current in Cultured Ovine Tracheal Epithelial (OTE) Cell Short Circuit Current (Isc)

The aim of this study was to investigate the effect of Bikunin (1-170) (See Example 17), on Isc in OTE cells in vitro. OTE cells were seeded onto 1.2 cm diameter, 0.4 um pore size Snapwell™ inserts (Costar UK). Cells were grown to confluence and mounted in modified Ussing chambers 3-5 days after placement on air-liquid interface. Inserts were immersed in Krebs buffer (KBR) solution and bubbled with 95% O₂/5% CO₂ warmed to 37° C.

After a 20 minute equilibration period, transepithelial potential difference was then clamped to 0 mV using a WPI EVC 4000 voltage clamp. Ag/AgCl electrodes were used to monitor Isc. Once a stable baseline was achieved (typically 30 mins), cells were treated with amiloride (10 uM). Once a response to amiloride was obtained, it was washed out with KBR solution. After return to baseline, Bikunin (1-170) described in Example 17 (25, 50 or 100 ug/mL) or PBS was added. 90 minutes following agent treatment amiloride (10 uM) was again added.

Results

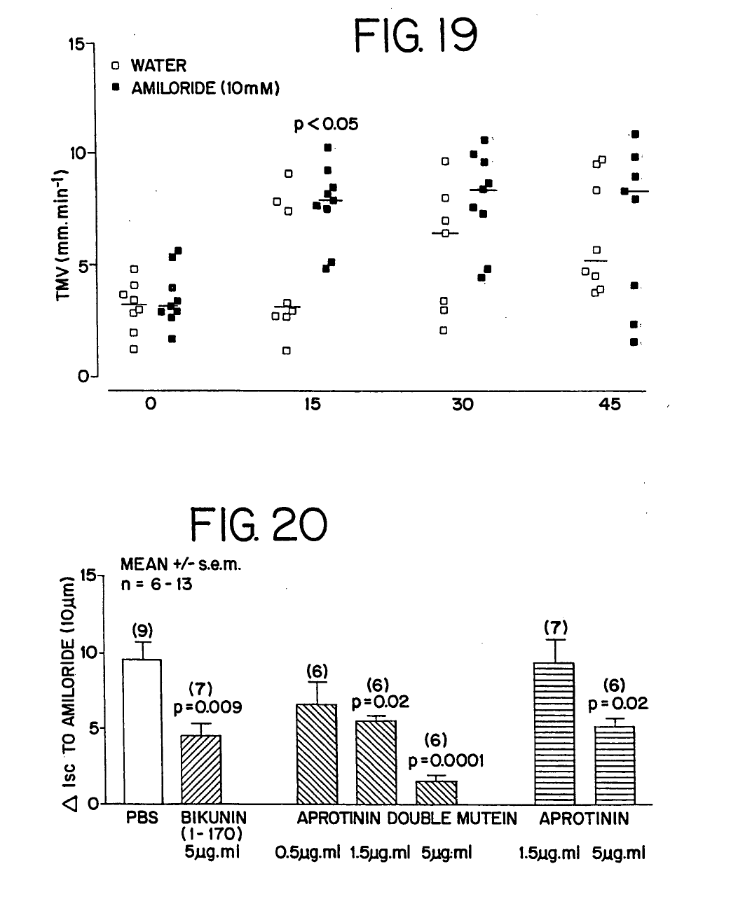

As shown in FIG. 24, Bikunin (1-170) (100 ug/mL) significantly inhibited sodium current in vitro in ovine tracheal epithelial cells over a 90 minute period.

### EXAMPLE 24

The effect of Bikunin (1-170) on Tracheal Potential Difference in Guinea Pigs Pretreated with LPS

Polymorphonuclear (PMN) leukocyte (neutrophil) dominated airway inflammation is often a feature of CF lung disease. In the guinea pig, exposure to an aerosol of E. coli lipopolysaccharide (LPS) induces a marked PMN influx in the bronchoalveolar Iavage fluid 24 hours post challenge. The aim of this study was to investigate the effect of Bikunin (1-170) described in Example 17 on tracheal potential difference in guinea pigs pre-exposed to an aerosol of LPS. Agents were delivered into the cephalad trachea by topical instillation. TPD was monitored for 60 minutes, 23 hours after exposure to LPS.

Materials/Reagents

Bikunin (1-170) was formulated in Hank's Balanced salt solution (HBSS). Amiloride was obtained from Sigma Chemicals and formulated in HBSS. Vehicle control was HBSS. Hypnorm (Fentanyl citrate 0.315 mg/mL and Fluanisone 10 mg/mL) was obtained from Janssen Animal Health and Hypnovel (Midazolam 5 mg.mL) was obtained from Roche. Male Dunkin-Hartley guinea pigs (600-700 g) were supplied by Harlan UK. Thermistor probes were obtained from Kane-May Ltd, UK.

Induction of PMN Influx

Individual animals were exposed to an aerosol of 0.03 mg/mL LPS or PBS for 10 minutes.

Preparation of Guinea Pig for Measurement to Tracheal Potential Difference

23.4 hours following LPS treatment, guinea pigs were anaesthetised with Hypnorm and Hypnovel and immobilised in a supine position. A ventral midline incision was made from the lower jaw to the clavicles. Using blunt dissection a length of trachea was exposed and bisected at the upper edge of the sternum. The external jugular vein was exposed and cannulated. The caudal part of the trachea was then cannulated to allow the animal to spontaneously breath room air. The animal was then placed supine and body temperature was maintained at 37° C. by manual adjustment of a heat lamp. Rectal temperature was monitored with a thermistor probe.

20 minutes following induction of anaesthesia, Bikunin (50 ug/mL) or amiloride (100 uM) was topically instilled into the cephalad trachea. The tracheal agar electrode was then inserted into the cephalad trachea and tracheal potential difference was measured for 60 minutes. The reference electrode was placed under the cephalad trachea in contact with the trachea cartilage. The wound site was covered to prevent drying.

Results

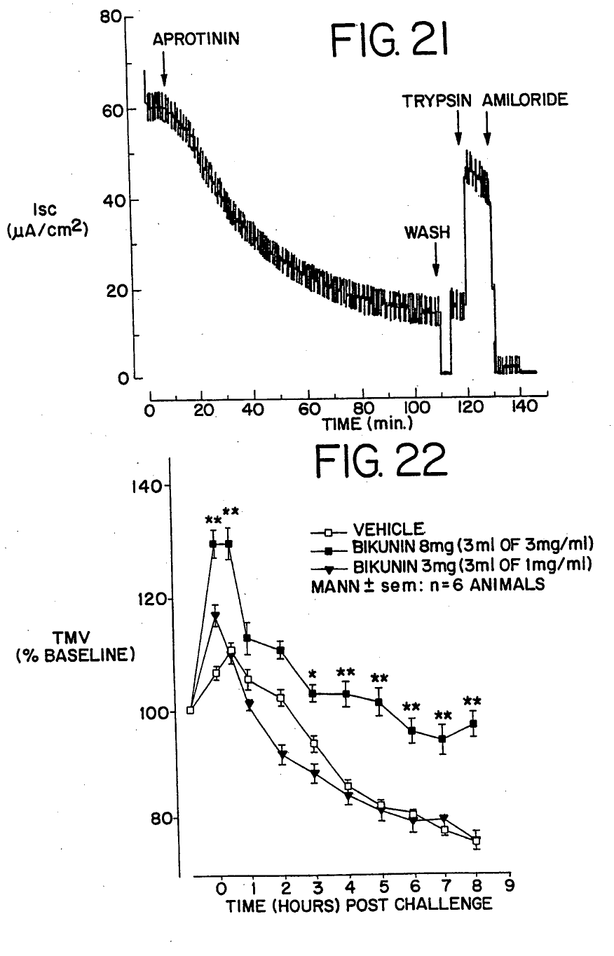

As shown in FIG. 25(a), exposure to LPS caused a significant PMN influx. Bikunin significantly inhibited potential difference in guinea pigs pre-exposed to LPS, as shown in FIG. 25(b).

### EXAMPLE 25

The effect of Aprotinin Double Mutein on Tracheal Mucus Velocity in the Guinea Pig

The aim of this study was to investigate the effect of Aprotinin double mutein described in Example 16 on guinea pig tracheal mucus velocity 1.5 hours post treatment. This agent was delivered into the cephalad trachea by topical instillation. TMV was monitored 1.5 hours later for 60 minutes.

Materials/Reagents

Aprotinin double mutein (see Example 16) was obtained from Biotechnologie, Bayer AG, Germany USA and formulated in Hank's Balanced salt solution (HBSS). Hypnorm (Fentanyl citrate 0.315 mg/mL and Fluanisone 10 mg/mL) was obtained from Janssen Animal Health and Hypnovel (Midazolam 5 mg/mL) was obtained from Roche. Male Dunkin-Hartley guinea pigs (600-750 g) were supplied by Harlan UK. Thermistor probes were obtained from Kane-May Ltd., UK.

Induction of Anaesthesia:

Animals were anaesthetised using halothane. Once a satisfactory level of anaesthesia was induced a small incision was made below the lower jaw. The trachea was exposed and 100 ul vehicle or Aprotinin double mutein (10 ug) was injected into the airway lumen using a needle and syringe. Once injected the skin overlying the tracheal injection site was repaired. The animals were then allowed to recover.

Measurement of Tracheal Mucus Velocity

Tracheal mucus velocity (TMV) was monitored using a lead collimated minature Beta-particle detector probe arranged to detect the radioactivity emitted from an injected aliquot of phosphorus-32-labelled Saccharomyces cerevisiae as it was transported on the tracheal mucociliary layer of an anaesthetised guinea pig (Newton and Hall 1998). 70 minutes following instillation of test agent, guinea pigs were anaesthestised for a second time with hypnorm and hypnovel and immobilised in a supine position. The first TMV measurement was made 20 minutes afterwards.

Results:

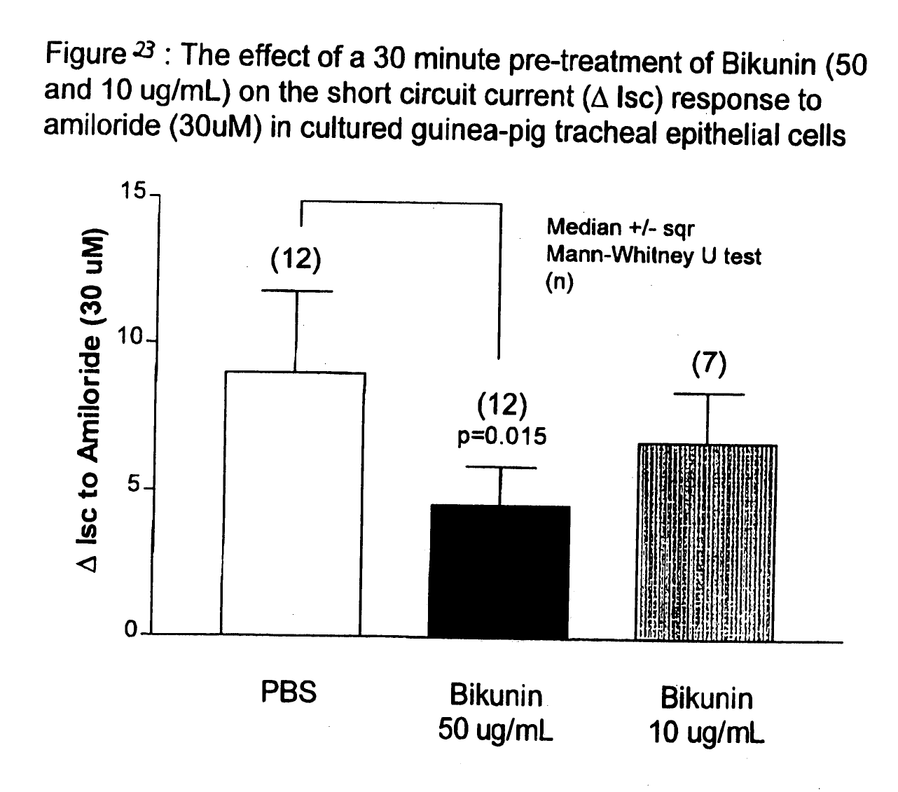

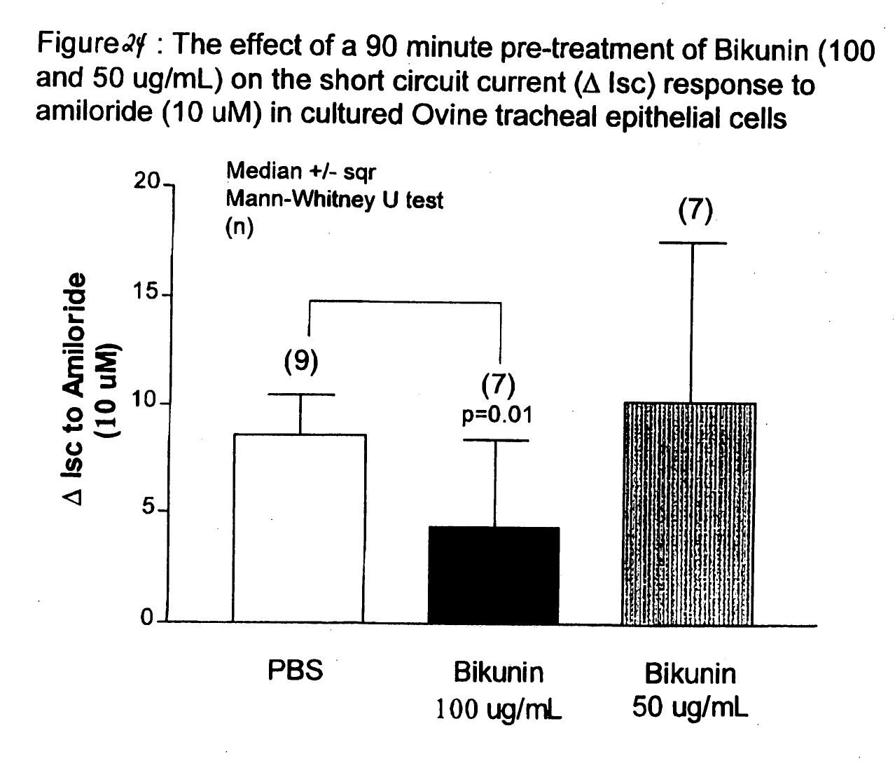

As shown in FIG. 26, Aprotinin double mutein (10 ug) increased TMV in vivo in the guinea pig, relative to HBSS, over a sustained period of 1.5 to 2.5 hours following administration.

### EXAMPLE 26

Bikunin (1-170) Decreases Sodium Current in Cultured Human Cystic Fibrosis Bronchial Epithelial Cell Short Circuit Current (Isc) In Vitro

HBE cells were isolated from CF patient lung transplant tissue and grown in collagen coated flasks for one week. The cells were then passaged and seeded onto collagen coated Costar Transwell filters (0.33 cm²) and grown in DMEM/F12 media supplemented with 2% Ultroser G. Cells were grown at an air liquid interface and used 2 to 4 weeks after seeding.

Cells on the Transwell filters were mounted in modified Costar Ussing chambers and studied under Isc conditions. Baseline Isc values were recorded (0 to 20 minutes), and then bikunin (1-170) (see Example 17) (10 ug/mL in PBS) was added to the apical side. Isc was recorded for 90 minutes and then amiloride (10 uM) was added and Isc was recorded for a further 10 minutes. The apical bath fluid was then exchanged with fresh buffer, and Isc was recorded for a further 15 minutes. Trypsin (1 uM) was added to the apical side and Isc recorded for 15 minutes. Amiloride (10 uM) was then added to the apical side and Isc was recorded for a further 10 minutes.

Results

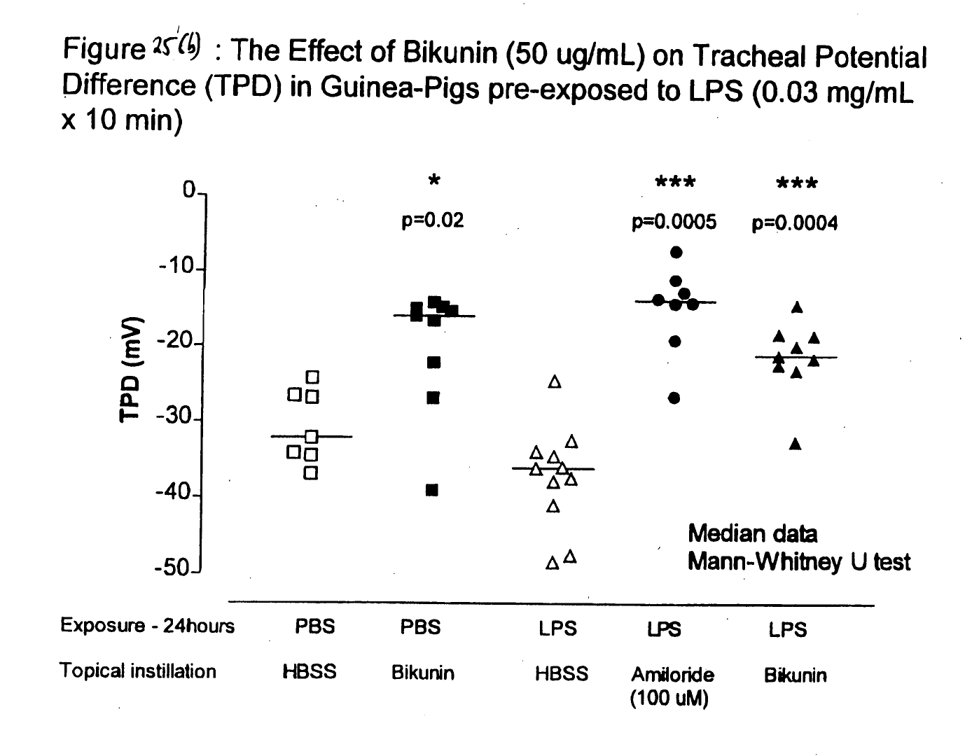

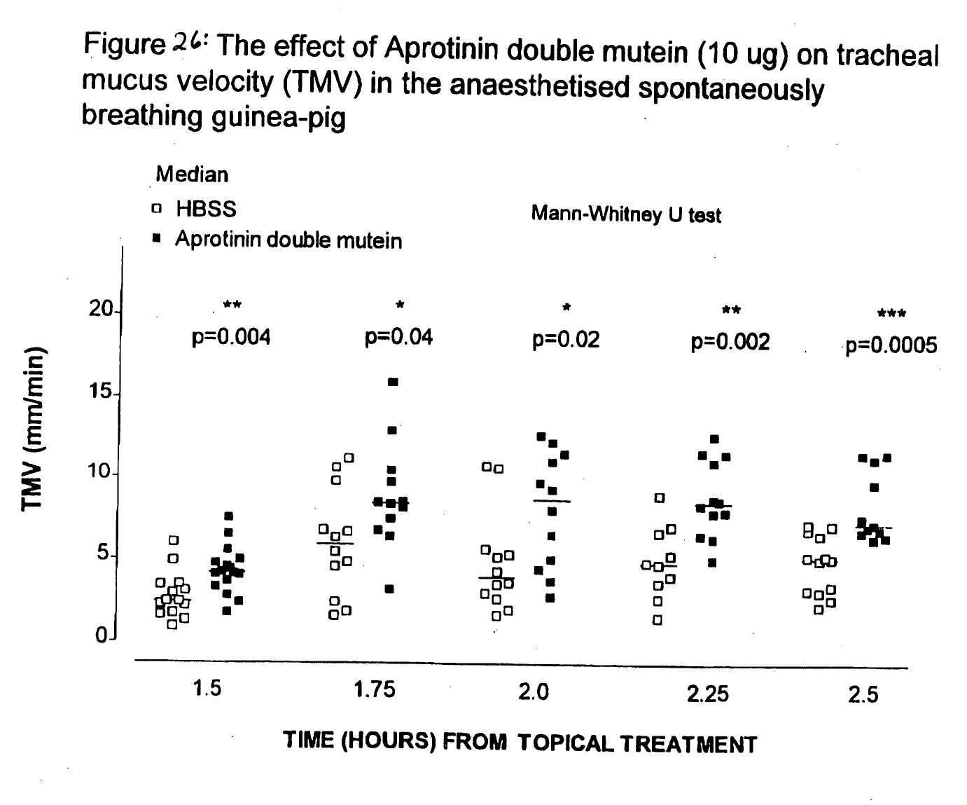

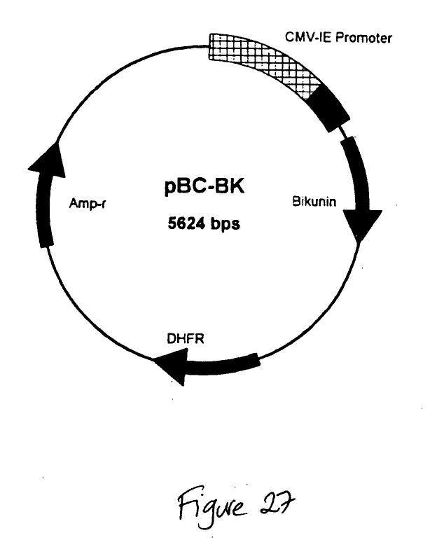

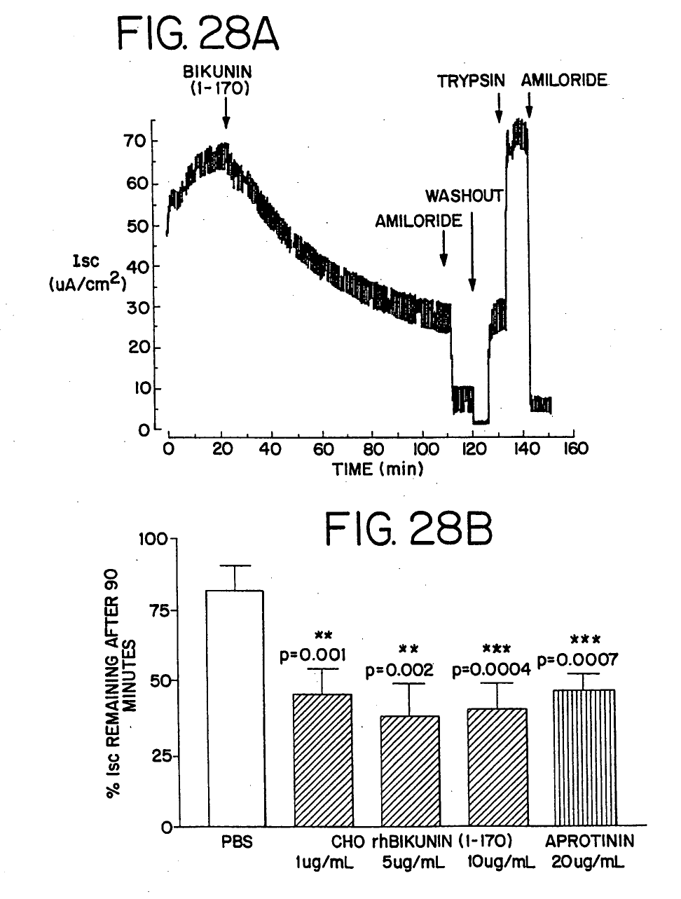

As shown in FIG. 28(a), bikunin (1-170) (10 ug/mL in PBS) inhibited Isc in vitro in human CF bronchial epithelial cells over a 90 minute period. Isc was further reduced by addition of amiloride (10 uM). On washout, Isc was increased back to the current achieved before the addition of amiloride. After a further 15 minutes, the apical surface was treated with trypsin (1 uM) and this further increased Isc to the baseline current level (i.e. that observed at 20 minutes). Finally, addition of amiloride (10 uM) inhibited the majority of the current, and demonstrated that the changes in Isc were the result of changes in sodium dependent current.

FIG. 28(b) shows that bikunin (1-170) at 1, 5, and 10 ug/mL, and aprotinin at 20 ug/mL inhibited Isc at 90 minutes after apical application to human CF bronchial epithelial cells in vitro.

Although certain embodiments of the invention have been described in detail for the purpose of illustration, it will be readily apparent to those skilled in the art that the methods and formulations described herein may be modified without departing from the spirit and scope of the invention. Accordingly, the invention is not limited except as by the appended claims.

We claim:

1. A method for accelerating the rate of mucociliary clearance in a subject in need thereof, comprising administering to the subject an effective amount of a protein as set forth in SEQ ID NO:52 and a physiological carrier, thereby accelerating the rate of mucociliary clearance.

2. The method of claim 1, wherein the effective amount is at least about 3 mg.

3. The method of claim 1, wherein the effective amount is about 3 to 9 mg.

4. The method of claim 1, wherein the effective amount is about 3 or 9 mg.

5. The method of claim 1, wherein the protein is administered to the lung airways.

6. The method of claim 1, wherein the protein is administered directly by aerosolization.

7. The method of claim 1, wherein the protein is administered directly as an aerosol solution into the subject's respiratory tract.

8. The method of claim 4, wherein the aerosol solution includes respirable particles ranging in size from about 1 to about 10 microns.

9. The method of claim 4, wherein the aerosol solution includes respirable particles ranging in size from about 1 to about 5 microns.

10. The method of claim 4, wherein the aerosol solution is delivered to the subject by a pressure driven nebulizer or administered as dry powder.

11. The method of claim 4, wherein the aerosol solution is delivered to the subject by an ultrasonic nebulizer.

12. The method of claim 4, wherein the aerosol solution is delivered to the subject by a non-toxic propellant.

13. The method of claim 1, wherein the carrier is a member selected from the group consisting of a physiologically buffered solution, an isotonic saline, normal saline, and combinations thereof.

14. The method of claim 1, wherein the protein is glycosylated.

15. The method of claim 1, wherein the protein inhibits a sodium channel.

16. The method of claim 1, wherein the channel is an epithelial sodium channel.

17. The method of claim 1, wherein the protein increases tracheal mucus velocity (TMV) in the subject.
